(19) United States
(12) Patent Application Publication
(10) Pub. No.: US 2024/0254169 A1
(43) Pub. Date: Aug. 1, 2024

(54) PROTEIN AND PEPTIDE DELIVERY SYSTEMS AND METHODS FOR MAKING AND USING THEM

(71) Applicants: San Diego State University (SDSU) Foundation, dba San Diego State University Research Foundation, San Diego, CA (US); J. CRAIG VENTER INSTITUTE, La Jolla, CA (US); ETH ZURICH, Zurich (CH)

(72) Inventors: Nicholas J. SHIKUMA, San Diego, CA (US); Sinem BEYHAN, La Jolla, CA (US); Martin PILHOFER, Zurich (CH); Charles ERICSON, Zurich (CH)

(21) Appl. No.: 17/294,656

(22) PCT Filed: Nov. 15, 2019
(86) PCT No.: PCT/US19/61839

§ 371 Date: May 17, 2021

Related U.S. Application Data
(60) Provisional application No. 62/844,988, filed on May 8, 2019.
(60) Provisional application No. 62/768,240, filed on Nov. 16, 2018.

(51) Int. Cl. C07K 14/195; C12N 15/88

(52) U.S. Cl. CPC C07K 14/195; C12N 15/88; C07K 2319/00

(57) ABSTRACT

Provided are compositions, kits, and methods for delivering a proteinaceous cargo, or a protein or a peptide, or a drug or a marker, to or into a cell or to an individual in need thereof. Methods comprise use of: a substantially purified or isolated bacterial Contractile Injection Systems (CIS) or Metamorphosis Associated Contractile structure; a recombinant bacterial Contractile Injection Systems (CIS) or Metamorphosis Associated Contractile structure (MACs); a liposome or lipid-comprising nanoparticle incorporating or expressing on its outer surface the substantially purified or isolated bacterial CIS or MACs, or the recombinant bacterial CIS or MACs; a protoplast or a spheroplast incorporating or expressing on its outer surface the substantially purified or isolated bacterial CIS or MACs, or the recombinant bacterial CIS or MACs; a cell expressing on its extracellular surface the substantially purified or isolated bacterial CIS or MACs, or the recombinant bacterial CIS or MACs.

### RELATED APPLICATIONS

This Patent Convention Treaty (PCT) International Application claims the benefit of priority to U.S. Provisional Application Ser. No. (U.S. Ser. No.) 62/768,240, filed Nov. 16, 2018; and U.S. Ser. No. 62/844,988, filed May 8, 2019. The aforementioned applications are expressly incorporated herein by reference in its entirety and for all purposes.

### STATEMENT AS TO FEDERALLY SPONSORED RESEARCH

This invention was made with government support under Department of Defense, Office of Naval Research (ONR) grant nos. N00014-17-1-2677; N00014-14-1-0340, and N00014-16-1-2135; and, NIH, NIDCD grant no. 1R21DC013180-01A1. The government has certain rights in the invention.

### TECHNICAL FIELD

This invention generally relates to microbiology and bioengineering. In alternative embodiments, provided are compositions and methods for delivering a proteinaceous cargo, or a polypeptide or peptide, to or into a cell, e.g., a eukaryotic cell such as a mammalian or a human cell, or to a plant cell, or to an individual in need thereof. In alternative embodiments, methods as provided herein comprise use of: (i) a substantially purified or isolated bacterial Contractile Injection System (CIS) or a Metamorphosis Associated Contractile structure (MAC); (ii) a recombinant bacterial Contractile Injection Systems (CIS) or Metamorphosis Associated Contractile structure (MAC); (iii) a liposome or lipid-comprising nanoparticle incorporating or expressing on its outer surface the substantially purified or isolated bacterial CIS or MACs, or the recombinant bacterial CIS or MACs; (iv) a protoplast or a spheroplast incorporating or expressing on its outer surface the substantially purified or isolated bacterial CIS or MACs, or the recombinant bacterial CIS or MACs; (v) a cell expressing on its extracellular surface the substantially purified or isolated bacterial CIS or MACs, or the recombinant bacterial CIS or MACs, wherein the CIS or MACs further comprise or have contained therein the proteinaceous cargo, or the protein or peptide; and, contacting the CIS or MACs with the cell, e.g., a eukaryotic cell such as a mammalian or a human cell, under conditions wherein the CIS or MACs contact or interact with the cell, e.g., eukaryotic cell, thereby delivering the proteinaceous cargo, or the protein or peptide to or into the cell, e.g., a eukaryotic cell, or to the individual in need thereof.

### BACKGROUND

Many bacteria interact with target organisms using syringe-like structures called Contractile Injection Systems (CIS). CIS structurally resemble headless bacteriophages and share evolutionarily related proteins such as the tail tube, sheath, and baseplate complex. Recent evidence shows that CIS are specialized to puncture membranes and often deliver effectors to target-cells. In many cases, CIS mediate trans-kingdom interactions between bacteria and eukaryotes, however the effectors delivered to target cells and their mode of action are often unknown.

A CIS mediating the beneficial relationship between the gram-negative bacterium Pseudoalteramonas luteoviolacea and marine tubeworm Hydroides elegans was recently characterized (Shikuma et al., 2014, 2016); and this CIS was named “Metamorphosis Associated Contractile structure” (MACs), because they stimulate the metamorphosis of Hydroides (Shikuma et al., 2014). While MACs provide an example of CIS-eukaryote interactions, the range of hosts targeted by CIS like MACs as well as the identity and mode of action of effectors that mediate these interactions remain poorly understood.

P. luteoviolacea produces arrays of Metamorphosis Associated Contractile structures (MACs) that induce the metamorphosis of Hydroides larvae (Huang et al., 2012; Shikuma et al., 2014). MACs are an example of a Contractile Injection System (CIS); macromolecular machines that are specialized to puncture membranes and often deliver proteinaceous effectors into target cells (Basler et al., 2012; Taylor et al., 2018). Like other CIS, MACs are evolutionarily related to the tails of bacteriophages (bacterial viruses) and are composed of an inner tube (gp19) surrounded by a contractile sheath, a tail-spike and baseplate complex (Shneider et al., 2013).

While CIS are known to deliver effectors to host cells, the method of delivery by injecting a protein residing within the inner tube lumen remains unconfirmed. For example, an amorphous density inside the inner tube of the Antifeeding prophage (Afp) was attributed to either the toxin payload or a tape-measuring protein (Heymann et al., 2013); the Pnf toxin was shown to physically associate with Photorhabdus Virulence Cassettes (PVCs) (Vlisidou et al., 2019). While certain classes of effectors are found to interact with the inner tube (gp19) and are likely loaded and released post-firing by the tube dissociation in the target cytoplasm (Silverman et al., 2013), no effector has ever been directly observed to be loaded within the inner tube of a CIS.

### SUMMARY

In alternative embodiments, provided are recombinant bacterial Contractile Injection Systems (CIS) or Metamorphosis Associated Contractile structures (MACs),

wherein the recombinant bacterial CIS or MACs comprise or have contained therein a proteinaceous cargo, or a heterologous protein or peptide,

and optionally the proteinaceous cargo, or a heterologous protein or peptide comprises a Metamorphosis-Inducing Factor 1 (Mif1) protein, or is linked to a Mif1 (optionally is chemically linked or electrostatically linked), or is a fusion or a recombinant protein comprising a Mif1,

wherein optionally the Mif1 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:1, or between about 80% to 100% sequence identity to SEQ ID NO:1,

or optionally the Mif1 protein comprises a sequence having at least about 85%, 86%, 87%, 88%, 89%, 90%, 91%, 92%, 93%, 94%, 95%, 96%, 97%, 98% or 100% sequence identity to SEQ ID NO:2, or between about 80% to 100% sequence identity to SEQ ID NO:2.

In alternative embodiments, provided are liposomes or lipid-comprising nanoparticles incorporating or expressing on its outer surface a substantially purified or isolated bacterial CIS or MACs, or a recombinant bacterial CIS or MACs,
wherein the substantially purified or isolated bacterial CIS, or the recombinant bacterial CIS or MACs, comprise or have contained therein a proteinaceous cargo, or a heterologous protein or peptide,

and optionally the proteinaceous cargo, or a heterologous protein or peptide comprises a Metamorphosis-Inducing Factor 1 (Mif1) protein, or is linked to a Mif1 (optionally is chemically linked or electrostatically linked), or is a fusion or a recombinant protein comprising a Mif1,

wherein optionally the Mif1 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:1, or between about 80% to 100% sequence identity to SEQ ID NO:1,

or optionally the Mif1 protein comprises a sequence having at least about 85%, 86%, 87%, 88%, 89%, 90%, 91%, 92%, 93%, 94%, 95%, 96%, 97%, 98% or 100% sequence identity to SEQ ID NO:2, or between about 80% to 100% sequence identity to SEQ ID NO:2.

In alternative embodiments, provided are protoplasts or spheroplasts incorporating or expressing on its outer surface a substantially purified or isolated bacterial CIS or MACs, or a recombinant bacterial CIS or MACs,

wherein the substantially purified or isolated bacterial CIS, or the recombinant bacterial CIS or MACs, comprise or have contained therein a proteinaceous cargo, or a heterologous protein or peptide,

and optionally the proteinaceous cargo, or a heterologous protein or peptide comprises a Metamorphosis-Inducing Factor 1 (Mif1) protein, or is linked to a Mif1 (optionally is chemically linked or electrostatically linked), or is a fusion or a recombinant protein comprising a Mif1,

wherein optionally the Mif1 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:1, or between about 80% to 100% sequence identity to SEQ ID NO:1,

or optionally the Mif1 protein comprises a sequence having at least about 85%, 86%, 87%, 88%, 89%, 90%, 91%, 92%, 93%, 94%, 95%, 96%, 97%, 98% or 100% sequence identity to SEQ ID NO:2, or between about 80% to 100% sequence identity to SEQ ID NO:2.

In alternative embodiments, provided are cells expressing on its extracellular surface a substantially purified or isolated bacterial CIS or MACs, or a recombinant bacterial CIS or MACs, wherein the CIS or MACs are heterologous to the cell, wherein the cell is a microbial cell or a eukaryotic cell, and optionally the microbial cell is a bacterial cell,

and the substantially purified or isolated bacterial CIS, or the recombinant bacterial CIS or MACs, comprise or have contained therein a proteinaceous cargo, or a heterologous protein or peptide,

and optionally the proteinaceous cargo, or a heterologous protein or peptide comprises a Metamorphosis-Inducing Factor 1 (Mif1) protein, or is linked to a Mif1 (optionally is chemically linked or electrostatically linked), or is a fusion or a recombinant protein comprising a Mif1,

wherein optionally the Mif1 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:1, or between about 80% to 100% sequence identity to SEQ ID NO:1,

or optionally the Mif1 protein comprises a sequence having at least about 85%, 86%, 87%, 88%, 89%, 90%, 91%, 92%, 93%, 94%, 95%, 96%, 97%, 98% or 100% sequence identity to SEQ ID NO:2, or between about 80% to 100% sequence identity to SEQ ID NO:2.

In alternative embodiments, provided are methods for delivering a proteinaceous cargo, or a protein or a peptide, or a drug or a marker, to a cell, e.g., a eukaryotic cell such as a mammalian or a human cell or a plant cell, or to an individual in need thereof, comprising:

(a) providing a formulation or composition comprising:
(i) a substantially purified or isolated bacterial Contractile Injection Systems (CIS) or Metamorphosis Associated Contractile structure (MACs);
(ii) a recombinant bacterial Contractile Injection Systems (CIS) or Metamorphosis Associated Contractile structure (MACs);
(iii) a liposome or lipid-comprising nanoparticle incorporating or expressing on its outer surface the substantially purified or isolated bacterial CIS or MACs, or the recombinant bacterial CIS or MACs;
(iv) a protoplast or a spheroplast incorporating or expressing on its outer surface the substantially purified or isolated bacterial CIS or MACs, or the recombinant bacterial CIS or MACs;
(v) a cell expressing on its extracellular surface the substantially purified or isolated bacterial CIS or MACs, or the recombinant bacterial CIS or MACs,
   wherein optionally the cell is a microbial cell or a eukaryotic cell, a mammalian or a human cell, and optionally the microbial cell is a bacterial cell; or
(vi) any combination of (i) to (v),

wherein the formulation or composition further comprises the proteinaceous cargo, or the protein or peptide, or the substantially purified or isolated bacterial CIS or MACs, or the recombinant bacterial CIS or MACs, comprise or have contained therein the proteinaceous cargo, or the protein or peptide,

and optionally the proteinaceous cargo, or a heterologous protein or peptide comprises a Metamorphosis-Inducing Factor 1 (Mif1) protein, or is linked to a Mif1 (optionally is chemically linked or electrostatically linked), or is a fusion or a recombinant protein comprising a Mif1,

wherein optionally the Mif1 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:1, or between about 80% to 100% sequence identity to SEQ ID NO:1,

or optionally the Mif1 protein comprises a sequence having at least about 85%, 86%, 87%, 88%, 89%, 90%, 91%, 92%, 93%, 94%, 95%, 96%, 97%, 98% or 100% sequence identity to SEQ ID NO:2, or between about 80% to 100% sequence identity to SEQ ID NO:2,

and optionally the protein or peptide is heterologous to the cell, and

(b) contacting the formulation or composition with cell, optionally a eukaryotic or a plant cell, under conditions wherein the formulation or composition, or the proteinaceous cargo, or the protein or peptide, contacts or interacts with the cell, e.g., a eukaryotic cell such as a mammalian or a human cell or a plant cell, thereby delivering the proteinaceous cargo, or the protein or peptide to or into the cell, e.g., a eukaryotic cell such as a mammalian or a human cell.

In alternative embodiments of methods as provided herein:
the proteinaceous cargo, or the protein or peptide, comprises or is an antibody, an enzyme, a drug, a marker, a detectable moiety, or an active biological agent;
the contacting of the formulation or composition with the cell, e.g., a eukaryotic cell such as a mammalian or a human cell, is in vitro, ex vivo, or in vivo; or
the eukaryotic cell is a mammalian, a human or an animal cell.

In alternative embodiments, provided are kits comprising: a formulation or composition comprising:
(i) a substantially purified or isolated bacterial Contractile Injection Systems (CIS) or Metamorphosis Associated Contractile structure (MACs);
(ii) a recombinant bacterial Contractile Injection Systems (CIS) or Metamorphosis Associated Contractile structure (MACs);
(iii) a liposome or lipid-comprising nanoparticle incorporating or expressing on its outer surface the substantially purified or isolated bacterial CIS or MACs, or the recombinant bacterial CIS or MACs;
(iv) a protoplast or a spheroplast incorporating or expressing on its outer surface the substantially purified or isolated bacterial CIS or MACs, or the recombinant bacterial CIS or MACs;
(v) a cell expressing on its extracellular surface the substantially purified or isolated bacterial CIS or MACs, or the recombinant bacterial CIS or MACs,
   wherein optionally the cell is a microbial cell or a eukaryotic cell, and optionally the microbial cell is a bacterial cell; or
(vi) any combination of (i) to (v),

wherein optionally the formulation or composition further comprises a proteinaceous cargo, or a protein or a peptide, or the substantially purified or isolated bacterial CIS or MACs, or the recombinant bacterial CIS or MACs, comprise or have contained therein the proteinaceous cargo, or the protein or peptide,

and optionally the proteinaceous cargo, or a heterologous protein or peptide comprises a Metamorphosis-Inducing Factor 1 (Mif1) protein, or is linked to a Mif1 (optionally is chemically linked or electrostatically linked), or is a fusion or a recombinant protein comprising a Mif1,

wherein optionally the Mif1 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:1, or between about 80% to 100% sequence identity to SEQ ID NO:1,

or optionally the Mif1 protein comprises a sequence having at least about 85%, 86%, 87%, 88%, 89%, 90%, 91%, 92%, 93%, 94%, 95%, 96%, 97%, 98% or 100% sequence identity to SEQ ID NO:2, or between about 80% to 100% sequence identity to SEQ ID NO:2,

and optionally the kit further comprises instructions for practicing a method as provided herein.

In alternative embodiments, provided are uses of a formulation or composition or a kit as provided herein for delivering a proteinaceous cargo, a protein or peptide, a drug or a marker, to or into a cell, e.g., a eukaryotic cell such as a mammalian or a human cell, wherein optionally the delivery of the formulation or composition with the eukaryotic cell is in vitro, ex vivo, or in vivo.

In alternative embodiments, provided are formulations, compositions or kits as provided herein for use in delivering a proteinaceous cargo, a protein or peptide, a drug or a marker, to or into a cell, eukaryotic cell such as a mammalian or a human cell, wherein optionally the delivery of the formulation or composition with the eukaryotic cell is in vitro, ex vivo, or in vivo.

The details of one or more embodiments as provided herein are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The drawings set forth herein are illustrative of embodiments as provided herein and are not meant to limit the scope of the invention as encompassed by the claims.

Figures are described in detail herein.

FIG. 1A-H illustrates data showing that MACs cause cytotoxicity in Sf9 insect cells: FIG. 1A-C illustrates images of Sf9 cells after 48 hours incubation with MACs from wild-type P. luteoviolacea (WT), ΔmacB mutant strain, or extraction buffer; FIG. 1D-F illustrates images of live/dead staining with fluorescein diacetate (FDA) (live cells) and propidium iodide (PI) (dead cells); and, FIG. 1G-H graphically illustrates quantification of cell death (%) utilizing (FIG. 1G) trypan blue and (FIG. 1H) FDA/PI live-dead stain, as discussed in detail in Example 1, below.

FIG. 2A-O illustrates data showing that MACs require JF50_12610 to kill insect cells: FIG. 2A-J illustrate images of Sf9 cells after 48 h incubation with MACs from WT, ΔJF50_12590, ΔJF50_12595, ΔJF50_12600, ΔJF50_12605, ΔJF50_12610 (Δpne1), ΔJF50_12615, ΔJF50_12610 ::JF50_12610, ΔmacB strains, and extraction buffer; FIG. 2K-L graphically illustrate quantification of cell death (%) by trypan blue and FDA/PI live-dead staining, and FIG. 2M graphically illustrates (%) meta-morphosis (%) of Hydroides larvae in response to MACs from WT, ΔmacB or ΔJF50_12610 strains; Electron cryo-tomography images of MACs from (FIG. 2N) wild type and (FIG. 2O) ΔJF50_12610 strains show an ordered structure with extended and contracted tubes connected by a meshwork of tail fibers; as discussed in detail in Example 1, below.

FIG. 3A-K illustrate that JF50_12610 (Pne1) contains a functional nuclease domain that is required for insect cell killing:

FIG. 3A illustrates protein sequence alignment of Pne1 (JF50_12610), Spd1 and Sda1, where the Pne1 sequence is SEQ ID NO: 107; the spd1 sequence is SEQ ID NO:108; the sda1 sequence is SEQ ID NO:109; the consensus_aa sequence is SEQ ID NO:109; and the consensus_ss sequence is SEQ ID NO:110;

FIG. 3B illustrates an image of a SDS Page gel of purified wild type Pne1, Pne1-Glu328Ala, Gfp, and DNase1;

FIG. 3C-D illustrate representative 1% agarose gel of DNA co-incubated with Pne1, Pne1-Glu328Ala and Gfp at 37° C. for 2 hour (PCR product size, 1.5 Kbp) in the absence (FIG. 3C) or presence (FIG. 3D) of 20 mM EDTA;

FIG. 3E-K illustrate Live/Dead images of Sf9 insect cells after 48 hours incubation with MACs from (FIG. 3E) WT, (FIG. 3F) Δpne1, (FIG. 3G) pne1-Glu328A, (FIG. 3H) pne1-ΔNLS, (FIG. 3I) ΔmacB, and (FIG. 3J) MAC extraction buffer; and

FIG. 3K graphically illustrates quantification of cell death (%) by FDA/PI live-dead stain,

as discussed in detail in Example 1, below.

FIG. 4A-E illustrate data showing that MACs kill J774A.1 murine macrophages and killing is dependent on JF50_12610L FIG. 4A-D illustrate images of J774A. 1 cells after incubation with MACs from WT, ΔJF50_12610, ΔmacB or extraction buffer; and FIG. 4E graphically illustrates cell death as quantified by Lactate Dehydrogenase (LDH) release assay at 24 hours, as discussed in detail in Example 1, below.

FIG. 5 graphically illustrates the number of MAC arrays per μl between wild type and ΔJF50_12610 mutant using a hemocytometer and fluorescence microscopy, as discussed in detail in Example 1, below.

FIG. 6 shows in tabular for a list of recombinant bacterial strains produced, as discussed in detail in Example 1, below.

FIG. 7A-G illustrate data showing that two bacterial genes are important for inducing Hydroides metamorphosis and produce MACs lacking electron density within their inner tubes:

FIG. 7A graphically illustrates Metamorphosis (%) of Hydroides larvae in response to biofilms of P. luteoviolacea lacking JF50_12590, JF50_12595, JF50_12600, JF50_12605, JF50_12610 or JF50_12615 genes;

FIG. 7B graphically illustrates restoration of JF50_12605 and JF50_12615 into their native chromosomal loci restores function,

FIG. 7C-D illustrate representative images of the filled tube phenotype from wild type cells (FIG. 7C) and (FIG. 7D) empty tube phenotype from gene deletion mutants;

FIG. 7E-F graphically illustrate density plots of representative sections from FIG. 7C (FIG. 7E) and FIG. 7D (FIG. 7F), respectively; and,

FIG. 7G graphically illustrates the fraction of empty structures for the JF50_12590-JF50_12615 region and each gene deletion;

as discussed in detail in Example 2, below.

FIG. 8A-J illustrates images of the lumen of MACs from a ΔJF50_12615 mutant lacks electron density: FIG. 8A-D: Cross sectional (FIG. 8A and FIG. 8C) and longitudinal (FIG. 8B and FIG. 8D) slices through initial subtomogram averages (pixel size of 4.28 Å) displayed a clear density difference of the lumen of the inner tube from the wild type structures when compared to the JF50_12615 mutant structure; FIG. 8E-F illustrate an improved average (pixel size of 2.72 Å) of a mutant that exhibits a bigger number of extended structures reinforces the presence of densities inside the inner tube; docking of the homology model for the gp19 structure (FIG. 8H) supports the assignment of sheath, tube and cargo densities for the 3D models (FIG. 8G-J), as discussed in detail in Example 2, below. FIG. 9A-D illustrate data showing JF50_12615 is present within MAC complexes and JF50_12605 is required for JF50_12615′s association with the MAC complex: FIG. 9A shows mass spectrometry of MACs complexes. JF50_12615 was detected within MAC complexes while JF50_12605 was not; FIG. 9B-D graphically illustrates quantification of Bacterial Two Hybrid analyses showing interactions between JF50_12605, JF50_12615 and JF50_12680 proteins. JF50_12605 showed a strong interaction with itself and with JF50_12615, as discussed in detail in Example 2, below.

FIG. 10A-C illustrate data showing that JF50_12615 is sufficient for stimulating metamorphosis when delivered by electroporation:

FIG. 10A illustrates an image of an SDS page gel of purified JF50_12615, JF50_12605 and GFP;

FIG. 10B illustrates an image of a Western blot of purified JF50_12615 protein probed with a C-terminal anti-JF50_12615 peptide antibody; and,

FIG. 10AC graphically illustrates Metamorphosis (%) of Hydroides larvae 24 hours after electroporation with purified JF50_12615, JF50_12605 or GFP protein, as discussed in detail in Example 2, below.

FIG. 11A-B graphically illustrate data showing the Metamorphic response of Hydroides larvae to cell-free MAC extracts from wild type P. luteoviolacea and individual gene mutants: FIG. 11A graphically illustrates Metamorphosis (%) of Hydroides larvae 24 hours after exposure to extracted MACs from P. luteoviolacea wild type and mutants; FIG. 11B graphically illustrates Dose response curve of MAC extracts from WT (red), AmacB (blue), ΔJF50_12605 (green), and ΔJF50_12615 (purple) mutants, as discussed in detail in Example 2, below.

FIG. 12A-D illustrate images showing that both WT and ΔJF50_12590-12615 have structurally similar arrays: MAC arrays were present in both WT (FIG. 12A A-B) and ΔJF50_12590-12615 (FIG. 12A C-D) MAC extracts, as discussed in detail in Example 2, below.

FIG. 13A-F, illustrate images showing filled and empty phenotype in all studied gene mutant strains: FIG. 13A-F illustrate slices through illustrative tomograms displaying the observed phenotype for each mutant, as discussed in detail in Example 2, below.

FIG. 14, Figure S4, graphically illustrates data showing that protein Purification and JF50_12615 is unable to induce metamorphosis when added exogenously: Metamorphosis (%) of Hydroides larvae after being soaked in 250 ng/μl of purified GFP, JF50_12605, and JF50_12615 protein for 24 hours, as discussed in detail in Example 2, below.

FIG. 15: illustrates strains and plasmids used in Example 2, as discussed in detail in Example 2, below.

FIG. 16A-B schematically illustrate that Bacteroidales possess a distinct Contractile Injection System: FIG. 16A shows that Contractile Injection Systems are related to the contractile tails of bacteriophage; there are two main types of CIS; Type 6 Secretion Systems (T6SS) act from within a bacterial cell, while extracellular CIS (eCIS) are released by bacterial cell lysis and bind to target cells; FIG. 16B show unrooted phylogeny of CIS sheath protein sequences; BIS group with known Subtype-4 T6SS and eCIS (orange) and are distinct from known Subtype-1, Subtype-2 and Subtype-3 T6SS; Bacteria with BIS identified in this study are highlighted in red, as discussed in detail in Example 3, below.

FIG. 17A-B schematically illustrate that Bacteroidales produce three architectures of BIS: FIG. 17A shows a Synteny plot of BIS gene clusters in Bacteroides and Parabacteroides species compared to (FIG. 17B) those of P. luteoviolacea MACs, S. entomophilia Afp, Photorhabdus PVCs, and A. asiaticus Subtype-4 T6SS. Representative CIS gene cluster architectures are shown, with genes color coded according to function; genes with no significant sequence similarity at the amino acid level to any characterized proteins are colored light grey; sequence coordinates of all gene clusters are provided in Table S3, as discussed in detail in Example 3, below.

FIG. 18A-B schematically illustrate that BIS genes are abundant in human gut and mouth microbiomes: FIG. 18A graphically illustrates a coverage plot of BIS genes (Log10 of 1,000,000*hits/reads) in 8,320 microbiomes associated with mucosal tissue: gut, mouth, nose, and other (includes vaginal and skin tissues) from 300 healthy humans; and FIG. 18B schematically illustrates ten BIS genes are found more often together in human metagenomes (co-occurrence network); node size represents the number of hits for each protein across all runs; line weight represents the number of times any two proteins occurred together within a dataset, as discussed in detail in Example 3, below.

FIG. 19 graphically illustrates that BIS genes are expressed in vivo in humanized mice, and shows a coverage plot of BIS genes (normalized by number of reads and protein nt size) from stool metatranscriptomes of humanized mouse microbiomes, as discussed in detail in Example 3, below.

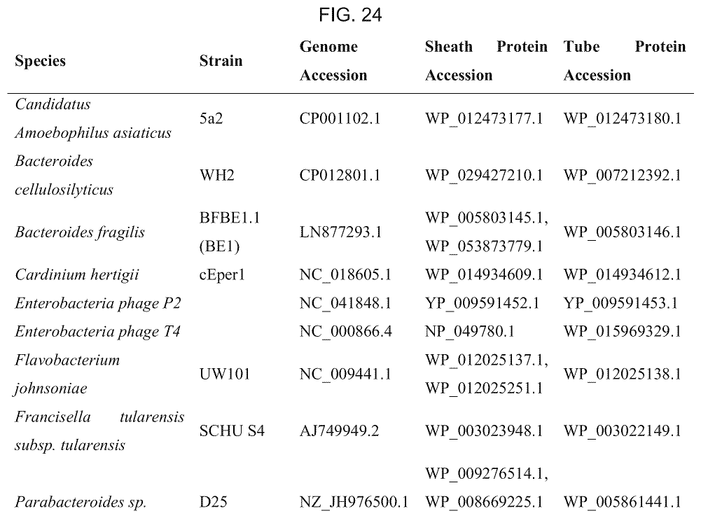

FIG. 20 graphically illustrates that BIS genes are present in the microbiomes of a majority (99%) of adult individuals from the United States and Europe; frequencies of 18 BIS proteins from fecal samples of 339 individuals. HMP n=215, from healthy North American individuals²; Qin n=124, from a study of European individuals²⁹. Protein hits are normalized by individual donor, as discussed in detail in Example 3, below.

FIG. 21 schematically illustrates the unrooted phylogeny of CIS tube protein sequences; BIS group with known T6SS-iv and CIS (orange) and are distinct from known T6SSⁱ and T6SSⁱⁱ present in human pathogens, and known ToSSⁱⁱⁱⁱ, characterized to mediate bacteria-bacteria interactions, as discussed in detail in Example 3, below.

FIG. 22 graphically illustrates that BIS genes are expressed during in vitro culture of B. cellulosilyticus WH2; relative abundance of RNA hits to 18 major genes of the BIS in B. cellulosilyticus WH2 culture in MM supplemented with 31 different simple and complex sugars, as discussed in detail in Example 3, below.

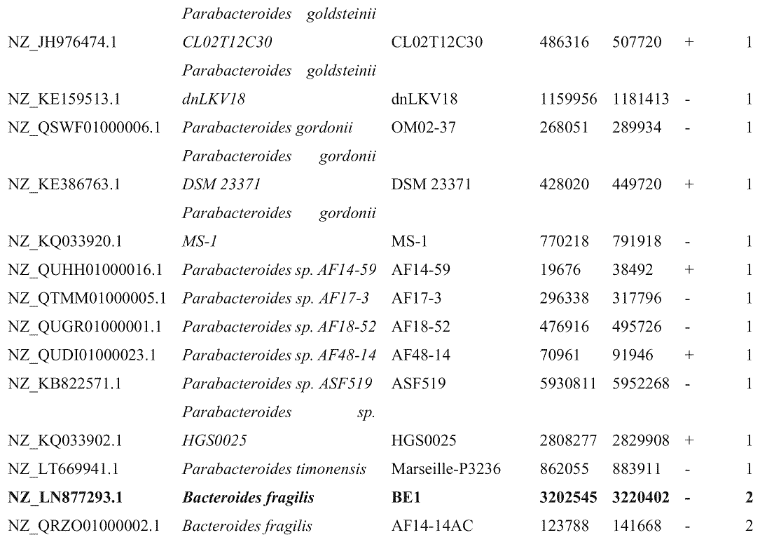

FIG. 23 illustrates Table S1, as discussed in detail in Example 3, below.

FIG. 24 illustrates Table S2, as discussed in detail in Example 3, below.

FIG. 25 illustrates Table S3, as discussed in detail in Example 3, below.

FIG. 26 illustrates Table S4, as discussed in detail in Example 3, below.

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, provided are compositions and methods for delivering a proteinaceous cargo, or a protein or a peptide, or a drug or a marker, to a eukaryotic cell such as a human cell, or to an individual in need thereof.

In alternative embodiments, methods as provided herein comprise use of: (i) a substantially purified or isolated bacterial Contractile Injection Systems (CIS) or Metamorphosis Associated Contractile structure; (ii) a recombinant bacterial Contractile Injection Systems (CIS) or Metamorphosis Associated Contractile structure (MACs); (iii) a liposome or lipid-comprising nanoparticle incorporating or expressing on its outer surface the substantially purified or isolated bacterial CIS or MACs, or the recombinant bacterial CIS or MACs; (iv) a protoplast or a spheroplast incorporating or expressing on its outer surface the substantially purified or isolated bacterial CIS or MACs, or the recombinant bacterial CIS or MACs; (v) a cell expressing on its extracellular surface the substantially purified or isolated bacterial CIS or MACs, or the recombinant bacterial CIS or MACs, wherein the CIS or MACs further comprise or have contained therein the proteinaceous cargo, or the protein or peptide; and, contacting the CIS or MACs with the eukaryotic cell under conditions wherein the CIS or MACs contact or interact with the eukaryotic cell, thereby delivering the proteinaceous cargo, or the protein or peptide to or into the eukaryotic cell.

Inventors have established an in vitro model to study a Contractile Injection Systems (CIS) called Metamorphosis Associated Contractile structures (MACs) that target and kill eukaryotic cells. We show that MACs can kill two eukaryotic cell lines, Fall Armyworm Sf9 cells and J774A.1 murine macrophage cells through the action of this newly identified MAC effector. This effector, termed Pne1, exhibits endonuclease activity and is necessary for the killing effect. Our results characterized a new mechanism of CIS-mediated bacteria-eukaryote interaction and provided the basis for the novel structures and methods as provided herein, which can be used as novel delivery systems for eukaryotic hosts.

Here for the first time we describe a CIS called Metamorphosis Associated Contractile structures (MACs) that possesses the ability to induce the metamorphic development of a model tubeworm. In this work, we established an in vitro model to study MACs and their effect on eukaryotic cells. We show that MACs can cause cell death in two eukaryotic cell lines, mouse derived macrophage and insect cell lines. Furthermore, we identified a MAC effector, termed Pne1, which contains endonuclease activity, that is responsible for causing that cell death.

### Generating and Manipulating Nucleic Acids

In alternative embodiments, nucleic acids used to practice methods as provided herein, or to make compositions or recombinant bacteria as provided herein, are made, isolated and/or manipulated by, e.g., cloning and expression of cDNA libraries, amplification of message or genomic DNA by PCR, and the like. The nucleic acids and genes used to practice this invention, including DNA, RNA, iRNA, antisense nucleic acid, cDNA, genomic DNA, vectors, viruses or hybrids thereof, can be isolated from a variety of sources, genetically engineered, amplified, and/or expressed/ generated recombinantly. Recombinant polypeptides generated from these nucleic acids can be individually isolated or cloned and tested for a desired activity. Any recombinant expression system or gene therapy delivery vehicle can be used, including e.g., viral (e.g., AAV constructs or hybrids) bacterial, fungal, mammalian, yeast, insect or plant cell expression systems or expression vehicles.

Alternatively, nucleic acids used to practice methods as provided herein, or to make compositions or recombinant bacteria as provided herein, can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids used to practice methods as provided herein, or to make compositions or recombinant bacteria as provided herein, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice methods as provided herein, or to make compositions or recombinant bacteria as provided herein, is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In alternative embodiments, a heterologous peptide or polypeptide joined or fused to a protein made by a method or a recombinant bacteria as provided herein can be an N-terminal identification peptide which imparts a desired characteristic, such as fluorescent detection, increased stability and/or simplified purification. Peptides and polypeptides made by a method or a recombinant bacteria as provided herein can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle WA). The inclusion of a cleavable linker sequences such as Factor Xa or enterokinase (Invitrogen, San Diego CA) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll (1993) DNA Cell. Biol., 12:441-53.

Nucleic acids or nucleic acid sequences used to practice embodiments as provided herein can be an oligonucleotide, nucleotide, polynucleotide, or to a fragment of any of these, to DNA or RNA of genomic or synthetic origin which may be single-stranded or double-stranded and may represent a sense or antisense strand, to peptide nucleic acid (PNA), or to any DNA-like or RNA-like material, natural or synthetic in origin. Compounds use to practice this invention include “nucleic acids” or “nucleic acid sequences” including oligonucleotide, nucleotide, polynucleotide, or any fragment of any of these; and include DNA or RNA (e.g., mRNA, rRNA, tRNA, iRNA) of genomic or synthetic origin which may be single-stranded or double-stranded; and can be a sense or antisense strand, or a peptide nucleic acid (PNA), or any DNA-like or RNA-like material, natural or synthetic in origin, including, e.g., iRNA, ribonucleoproteins (e.g., e.g., double stranded iRNAs, e.g., iRNPs). Nucleic acids or nucleic acid sequences used to practice embodiments as provided herein include nucleic acids or oligonucleotides containing known analogues of natural nucleotides. Nucleic acids or nucleic acid sequences used to practice embodiments as provided herein include nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156. Nucleic acids or nucleic acid sequences used to practice embodiments as provided herein include “oligonucleotides” including a single stranded polydeoxynucleotide or two complementary polydeoxynucleotide strands that may be chemically synthesized. Compounds use to practice this invention include synthetic oligonucleotides having no 5′ phosphate, and thus will not ligate to another oligonucleotide without adding a phosphate with an ATP in the presence of a kinase. A synthetic oligonucleotide can ligate to a fragment that has not been dephosphorylated.

In alternative aspects, methods and recombinant bacteria as provided herein comprise use of “expression cassettes” comprising a nucleotide sequences capable of affecting expression of the nucleic acid, e.g., a structural gene or a transcript (e.g., encoding a Contractile Injection Systems (CIS)) in a host compatible with such sequences. Expression cassettes can include at least a promoter operably linked with the polypeptide coding sequence or inhibitory sequence; and, in one aspect, with other sequences, e.g., transcription termination signals. Additional factors necessary or helpful in effecting expression may also be used, e.g., enhancers.

In alternative aspects, expression cassettes used to practice embodiments as provided herein also include plasmids, expression vectors, recombinant viruses, any form of recombinant “naked DNA” vector, and the like. In alternative aspects, a “vector” used to practice embodiments as provided herein can comprise a nucleic acid that can infect, transfect, transiently or permanently transduce a cell. In alternative aspects, a vector used to practice embodiments as provided herein can be a naked nucleic acid, or a nucleic acid complexed with protein or lipid. In alternative aspects, vectors used to practice embodiments as provided herein can comprise viral or bacterial nucleic acids and/or proteins, and/or membranes (e.g., a cell membrane, a viral lipid envelope, etc.). In alternative aspects, vectors used to practice embodiments as provided herein can include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and can include both the expression and non-expression plasmids. In alternative aspects, the vector used to practice embodiments as provided herein can be stably replicated by the cells during mitosis as an autonomous structure, or can be incorporated within the host's genome.

In alternative aspects, “promoters” used to practice this invention include all sequences capable of driving transcription of a coding sequence (e.g., for a Contractile Injection Systems (CIS)) in a cell, e.g., a bacterial cell. Thus, promoters used in the constructs of the invention include cis-acting transcriptional control elements and regulatory sequences that are involved in regulating or modulating the timing and/or rate of transcription of a gene. For example, a promoter used to practice this invention can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

### Metamorphosis-Inducing Factor 1 (Mif1) Proteins

In alternative embodiments, formulations or compositions as provided herein further comprise a proteinaceous cargo, or a protein or a peptide, or the substantially purified or isolated bacterial CIS or MACs, or the recombinant bacterial CIS or MACs, comprise or have contained therein the proteinaceous cargo, or the protein or peptide, and optionally the proteinaceous cargo, or a heterologous protein or peptide comprises a Metamorphosis-Inducing Factor 1 (Mif1) protein, or is linked to a Mif1 (optionally is chemically linked or electrostatically linked), or is a fusion or a recombinant protein comprising a Mif1, wherein optionally the Mif1 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:1, or between about 80% to 100% sequence identity to SEQ ID NO:1, or optionally the Mif1 protein comprises a sequence having at least about 85%, 86%, 87%, 88%, 89%, 90%, 91%, 92%, 93%, 94%, 95%, 96%, 97%, 98% or 100% sequence identity to SEQ ID NO:2, or between about 80% to 100% sequence identity to SEQ ID NO:2.

In alternative embodiments, the sequence identity is calculated using a sequence comparison algorithm consisting of a BLAST version 2.2.2 algorithm where a filtering setting is set to blastall-p blastp-d “nr pataa”-F F, and all other options are set to default. In alternative embodiments, protein and/or nucleic acid sequence homologies are calculated using any of the variety of sequence comparison algorithms and programs known in the art. Such algorithms and programs include, but are by no means limited to, TBLASTN, BLASTP, FASTA, TFASTA and CLUSTALW (Pearson and Lipman, Proc. Natl. Acad. Sci. USA 85(8):2444-2448, 1988; Altschul et al., J. Mol. Biol. 215(3):403-410, 1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

In alternative embodiments, the sequence identity (homology) is calculated using BLAST and BLAST 2.0 algorithms, which are described in Altschul et al., Nuc. Acids Res. 25:3389-3402, 1977 and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3 and expectations (E) of 10 and the BLOSUM62 scoring matrix (see Henikoff & Henikoff, Proc. Natl. Acad. Sci. USA 89:10915, 1989) alignments (B) of 50, expectation (E) of 10, M=5, N=−4 and a comparison of both strands.

In alternative embodiments, protein and nucleic acid sequence homologies (sequence identity) are evaluated using the Basic Local Alignment Search Tool (“BLAST”) In particular, five specific BLAST programs are used to perform the following task: (1) BLASTP and BLAST3 compare an amino acid query sequence against a protein sequence database; (2) BLASTN compares a nucleotide query sequence against a nucleotide sequence database; (3) BLASTX compares the six-frame conceptual translation products of a query nucleotide sequence (both strands) against a protein sequence database; (4) TBLASTN compares a query protein sequence against a nucleotide sequence database translated in all six reading frames (both strands); and (5) TBLASTX compares the six-frame translations of a nucleotide query sequence against the six-frame translations of a nucleotide sequence database.

SEQ ID NO: 1 is:
ATGCAACAACAAGAACAGGAGCAAGCTCCTACCTTTCAAAGTTATCCCAC
GCGCACTGCGTTATCAGTGAGCAATGCATTAGACTCTGATTCTGTGAGTGT
CGATCTACTCTCATCGGGCGTTGATGTTGCAAAGCGGTATGCCTCACAAGT
GGTTTGGGATCATTTTAATGGCGATGCAACAGCTAAGCTGATTATCTCAAG
TGTATTTAATTATGCGGTGACATCATTAAAGTCCTTGAACCCATGGGTGAC
GGCAATCTCACAAGCACTTTTACTTTTGGCAAAAGTTCCGCCTGGTGTTGT
TTCCGCCGTTTTATGGGCCATTGGAAAAATCTGGCTTTGGGCTGCAAATAA
ATTTTATAACGGTGGTTGGATAGCCGCTGCGTGGGGAGATATTGATGAGC
CATATATTTATCAATGGTTAAAAAAAGGCAGTGATGCACATGGGGCATTA
CGGGCACTCGTGGATGATTTAAAAGCTTGGGTTAAGTATATTCAAGATAA
GCTTGCCAGTAGCGTTGCACGTTTAATTGGTGTCTCGGATTCAAGTAGTGA
AGATGAGCAAAGCGATGAGCAACAAACGGATCAAGATGCACAAACATCA
CCTAATATAGTTGATAATAAGTTCATTTCGTTGGGGATAAACCAACCTGAA
TTATTGGACTGGGAAGAGGGGGCAAGTAAGCCCAAGCGCGCTGGACTAC
ATGGTACGGGTGTTGCCAAGGTCAATCTGCTTGGTCAGCAATTTGGTGGTG
ATATAGATGTAAAATTACCATTTGGCAGTGGCTGGGAAGTGGCTGTATCT
CCGGTTTTTGCAAGCCAACAAGGGGTGGGTTTTAGTGGTTATATTGAAGC
GCATCAAGTATTGGGCGATGAGCTGACCATTAATGAAGAAGGCCTTGCTC
GTTTTAAAGCAAGCATTGTTGGGCTAAACATTGCGAACAAGCGGGTTTAC
TCCGATTTAATGTCACTTGATTACAATAAGCAGCAAAAAGAAGTTCATTTT
GCAGGTGATGCACATGTGCCGCTTTGGGATAAAAAGAAACTGGATGGTGA
GTTTGATTTAAAATTGGATACTGCAGGTAAATTCAAATCAGGCAGGACCA
AAATCTCTTCTAAAGATACATTCGAAGTAATCCCCAAGTTTTTGTCTATCA
GCAACCCGAGTGGTGAAGTAGAAGTTAAAGAGGATGCTTCACCAGAGTTT
GAGGTGAGTACTGACGCTGCGCTTTACGGCTTACCCGCTGGTGTGAAAGC
ATCAATTACGCAAGCAAAAGTGATGTATCACGATGAGCAATTAGGCGGTG
AGGTTGAGCAGGCTGATGTGGAGATCCCAATTAGCAAACACACTACAATG
ACGCTGGCCATGACGAAGGCTAAGTTTACAAAAGATGCAATTGAAGCTGA
AAATGCTTTTATGGATCTCGATCATGATAATGCCAAAGTGCTTAATGAAAC
GGCCATTGTCGAGTCAGACTTTTTCTCTGGAAACTTCGATTTTGGAAAAGT
GTTTGAGCTTAAGCAGTTGAAAGTCCACCAAGGGTTAAACGCGCTTAAAT
TTGCTGGTGGTAAATTCTCCTACGAGAAGGATAGCAACAATGGTTTACAG
TTACTTAGAGCACGTATTTTAGGCCTTGAGGCTTACTATAATAGAAAAGAT
CGTGAAGGTGGGATCACTGGAGAATGGCACAAAGGGATTGATTTTCCGGC
TTTCTCTTTGGACTTTCCCGTTGCGGCTGGTGTTGCAGGAGTTGGAGTTGA
TATTACCGGGGGGTTTGAGTTTGGTGCGAGCATCGGCGCTAAGCTTAAAA
ATGAAGAGCAGCATAACACAGAGACACAAATGCTTTTCTCTGTGAATGGT
GCAGCTTCGGCCAGTGCCAAAGCTAAAGCGAGAATTGAGGCTGGTGCTTT
TGTGGGGGTCCCTTATCTTGCAAAGGTTCAAGGTGGTGTATTTGGTGAGAT
CCGAGGTGGTGTTGAAGGTAAGGTTGAAACAGAAGGGCGTTTGAAATACA
CGCGTGGCAAAGGCGGTGATAGCTTTGGTCAATTTGCCATTGATAGTGATT
ATCCAATGGAGGCGAGCTTTAGCCTAACCGGCTCATTAGAAGCGGAAGTA
GGTGCATCGATAAAAGCCAAAGTACTGACATTTGAAAAAGAAATTGCGTC
GGTATCTATTGGTGATTGGACACTGGGTGAGTATACGCTTAATGGTAAAA
TCAAAAAAGATCCTAATGGGAAAGGGTATATCGTTGAGCGCTCTAAAGGG
GAGTTTACCGAAGGCAAACCAAAACCTGCCGAGGTAACTAAGGAGGCAC
TCCCAGTAGATAAGTGGGTTAATCAGTTAGAAAAAGATCATACTCATATT
GAATACACCGAAGATGAAGCCAAAAGAAAGTTACCTGAGGTTGCACGTA
AAACGGTATATAGTCACCTGAGTCCAATGCAAAAAGAGGATGTAAGTGAG
CGATATACTCATTTGCTCAAGTTGATATCGCAGCAAAATAAGTTCACTGAA
ATTTATCGAGAAACCAAAGCTGATAGGGATAAGTTACCGACCAGTGGCAC
TTATATTTGGACCTCTAAAGTTTGGAATGAAGCGGTACAGCGCAAGAGTT
TCTGGATTTTTGAAATGAGTAAGAGTAAAGCAAAAGTGGCGGATAAGCTG
GATAAATATCACAAAACGCACTCTATTTTAGAGCGGAGACTGTTACTTGC
AAAGCTAGAAGAGATAGCCTCGGACTATCGCAAAAACCGCTCGAAAAAC
GCAGAAAATAAAGAAGCTGCCGGGGAGTTTTTAGAGAGTATTGAAAAAG
AAAGGCTCATTTTAATGTAA
SEQ ID NO: 2 is:
MQQQEQEQAPTFQSYPTRTALSVSNALDSDSVSVDLLSSGVDVAKRYASQVV
WDHFNGDATAKLIISSVFNYAVTSLKSLNPWVTAISQALLLLAKVPPGVVSAV
LWAIGKIWLWAANKFYNGGWIAAAWGDIDEPYIYQWLKKGSDAHGALRAL
VDDLKAWVKYIQDKLASSVARLIGVSDSSSEDEQSDEQQTDQDAQTSPNIVD
NKFISLGINQPELLDWEEGASKPKRAGLHGTGVAKVNLLGQQFGGDIDVKLP
FGSGWEVAVSPVFASQQGVGFSGYIEAHQVLGDELTINEEGLARFKASIVGLN
IANKRVYSDLMSLDYNKQQKEVHFAGDAHVPLWDKKKLDGEFDLKLDTAG
KFKSGRTKISSKDTFEVIPKFLSISNPSGEVEVKEDASPEFEVSTDAALYGLPAG
VKASITQAKVMYHDEQLGGEVEQADVEIPISKHTTMTLAMTKAKFTKDAIEA
ENAFMDLDHDNAKVLNETAIVESDFFSGNFDFGKVFELKQLKVHQGLNALKF
AGGKFSYEKDSNNGLQLLRARILGLEAYYNRKDREGGITGEWHKGIDFPAFS
LDFPVAAGVAGVGVDITGGFEFGASIGAKLKNEEQHNTETQMLFSVNGAASA
SAKAKARIEAGAFVGVPYLAKVQGGVFGEIRGGVEGKVETEGRLKYTRGKG
GDSFGQFAIDSDYPMEASFSLTGSLEAEVGASIKAKVLTFEKEIASVSIGDWTL
GEYTLNGKIKKDPNGKGYIVERSKGEFTEGKPKPAEVTKEALPVDKWVNQLE
KDHTHIEYTEDEAKRKLPEVARKTVYSHLSPMQKEDVSERYTHLLKLISQQN
KFTEIYRETKADRDKLPTSGTYIWTSKVWNEAVQRKSFWIFEMSKSKAKVAD
KLDKYHKTHSILERRLLLAKLEEIASDYRKNRSKNAENKEAAGEFLESIEKER
LILM

### Bacterial Growth Conditions

Any set of known growth conditions can be used to practice embodiments as provided herein, for example, as described in US 2016-0237398 A1, or WO/2015/058179; exemplary growth conditions and parameters are described in Example 1, below.

### Formulations

In alternative embodiments, provided are formulations, including pharmaceutical formulations, for delivering a proteinaceous cargo, a protein or peptide, a drug or a marker, to or into a eukaryotic cell, wherein optionally the delivery of the formulation or composition with the eukaryotic cell is in vitro, ex vivo, or in vivo. For example, in alternative embodiments, substantially purified or isolated bacterial CIS or MACs, or the recombinant bacterial CIS or MACs, or liposomes or lipid-comprising nanoparticles incorporating or expressing on their outer surface the substantially purified or isolated bacterial CIS or MACs, or the recombinant bacterial CIS or MACs, as provided herein, are formulated in sterile saline or buffered formulations. In alternative embodiments, formulations as provided herein comprise water, saline, a pharmaceutically acceptable preservative, a carrier, a buffer, a diluent, an adjuvant or a combination thereof.

In alternative embodiments formulations as provided herein are administered orally or rectally, or are formulated as a liquid, a food, a gel, a candy, an ice, a lozenge, a tablet, pill or capsule, or a suppository or as an enema formulation, or for any form of intra-rectal or intra-colonic administration.

In alternative embodiments, formulations are provided herein are administered or are delivered in vivo by any effective means appropriated for a particular treatment. For example, depending on the specific agent to be administered with (by carried by) a CIS or MAC-comprising formulations as provided herein, a suitable means can include oral, rectal, vaginal, nasal, pulmonary administration, or parenteral (including subcutaneous, intramuscular, intravenous and intradermal) infusion into the bloodstream. For parenteral administration, CIS or MAC-comprising formulations as provided herein can be formulated in a variety of ways. Aqueous solutions of the modulators can be encapsulated in polymeric beads, liposomes, nanoparticles or other injectable depot formulations known to those of skill in the art. In alternative embodiments, CIS or MAC-comprising formulations as provided herein are administered encapsulated in liposomes (see below). In alternative embodiments, depending upon solubility, compositions are present both in an aqueous layer and in a lipidic layer, e.g., a liposomic suspension. In alternative embodiments, a hydrophobic layer comprises phospholipids such as lecithin and sphingomyelin, steroids such as cholesterol, more or less ionic surfactants such a diacetylphosphate, stearylamine, or phosphatidic acid, and/or other materials of a hydrophobic nature.

In alternative embodiments, formulations are provided herein are formulated in any way and can be administered in a variety of unit dosage forms depending upon a desired result, e.g., a condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co., Easton PA (“Remington's”).

For example, in alternative embodiments, CIS or MAC-comprising formulations as provided herein are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice embodiments as provided herein can be in forms such as tablets, pills, powders, capsules, liquids, gels, syrups, slurries, suspensions, etc., suitable for in vivo, in vitro or ex vivo applications.

In practicing embodiments as provided herein, CIS or MAC-comprising formulations as provided herein can comprise a solution of compositions disposed in or dissolved in a pharmaceutically acceptable carrier, e.g., acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice embodiments as provided herein are sterile and can be manufactured to be generally free of undesirable matter. In one embodiment, these solutions and formulations are sterilized by conventional, well known sterilization techniques.

CIS or MAC-comprising formulations as provided herein as provided herein can comprise auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

CIS or MAC-comprising formulations as provided herein can be delivered by the use of liposomes. In alternative embodiments, by using liposomes, particularly where the liposome surface carries ligands specific for target cells or organs, or are otherwise preferentially directed to a specific tissue or organ type, one can focus the delivery of the CIS or MAC-comprising formulations, and thus an active agent, to or into a target cells in an in vivo, in vitro or ex vivo application.

CIS or MAC-comprising formulations as provided herein can be directly administered, e.g., under sterile conditions, to an individual (e.g., a patient) to be treated. The modulators can be administered alone or as the active ingredient of a pharmaceutical composition. Compositions and formulations as provided herein can be combined with or used in association with other therapeutic agents. For example, an individual may be treated concurrently with conventional therapeutic agents.

### Nanoparticles, Nanolipoparticles and Liposomes

Provided are nanoparticles, nanolipoparticles, vesicles and liposomal membranes comprising CIS or MAC-comprising formulations as provided herein. Provided are multilayered liposomes comprising compounds used to practice embodiments as provided herein, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice embodiments as provided herein.

Liposomes can be made using any method, e.g., as described in Park, et al., U.S. Pat. Pub. No. 20070042031, including the method of producing a liposome by encapsulating an active agent (e.g., CIS or MAC-comprising formulations as provided herein), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice embodiments as provided herein comprise a substituted ammonium and/or polyanions, e.g., for targeting delivery of a compound as provided herein, or a compound used to practice methods as provided herein, to a desired cell type or organ, e.g., brain, as described e.g., in U.S. Pat. Pub. No. 20070110798.

Provided are nanoparticles comprising compounds as provided herein, e.g., used to practice methods as provided herein in the form of active agent-containing nanoparticles (e.g., a secondary nanoparticle), as described, e.g., in U.S. Pat. Pub. No. 20070077286. In one embodiment, provided are nanoparticles comprising a fat-soluble active agent used to practice embodiments as provided herein, or a fat-solubilized water-soluble active agent to act with a bivalent or trivalent metal salt.

In one embodiment, solid lipid suspensions can be used to formulate and to deliver compositions used to practice embodiments as provided herein to mammalian cells in vivo, in vitro or ex vivo, as described, e.g., in U.S. Pat. Pub. No. 20050136121.

### Delivery Vehicles

In alternative embodiments, any delivery vehicle can be used to practice the methods as provided herein, e.g., to deliver CIS or MAC-comprising formulations as provided herein, to mammalian cells, e.g., in vivo, in vitro or ex vivo. For example, delivery vehicles comprising polycations, cationic polymers and/or cationic peptides, such as polyethyleneimine derivatives, can be used e.g. as described, e.g., in U.S. Pat. Pub. No. 20060083737.

In one embodiment, a dried polypeptide-surfactant complex is used to formulate CIS or MAC-comprising formulations as provided herein, e.g. as described, e.g., in U.S. Pat. Pub. No. 20040151766.

In one embodiment, compounds and compositions as provided herein, or a compound used to practice methods as provided herein, can be applied to cells using vehicles with cell membrane-permeant peptide conjugates, e.g., as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, e.g., as described in U.S. Pat. No. 5,846,743, describing transport-mediating peptides that are highly basic and bind to poly-phosphoinositides.

In one embodiment, electro-permeabilization is used as a primary or adjunctive means to deliver the composition to a cell, e.g., using any electroporation system as described e.g. in U.S. Pat. Nos. 7,109,034; 6,261,815; 5,874,268.

### Dosaging

In alternative embodiments, CIS or MAC-comprising formulations as provided herein, including pharmaceutical compositions, are administered for prophylactic and/or therapeutic treatments. In therapeutic applications, compositions are administered to a subject, e.g., a human in need thereof, in an amount of the agent sufficient to cure, alleviate or partially arrest the clinical manifestations and/or its complications (a “therapeutically effective amount”).

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. Dosage levels may range from about 0.01 mg per kilogram to about 100 mg per kilogram of body weight. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition. For example, in alternative embodiments, a substantially purified or isolated bacterial CIS or MACs is at least about 90%, 95%, 97%, 98%, 99% or 99.5%, or more pure, or is between about 85% and 99.5% pure, or having no more than about 10%, 9%, 8%, 7%, 6%, 5%, 4%, 3%, 2%, 1%, 0.5%, 0.1%, 0.05%, or 0.01% non-bacterial CIS or MAC elements.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the following examples; however, it is to be understood that the invention is not limited to such examples.

### EXAMPLES

### Example 1: Exemplary Methods and Compositions: Bacterial Phage Tail-Like Structure Kills Eukaryotic Cells by Injecting a Nuclease Effector

This example provides exemplary methods for making compositions and bacterial cells as provided herein.

To study the interaction between Metamorphosis Associated Contractile structures (MACs) and eukaryotic cells, we established an in vitro CIS-cell line interaction model with insect and mammalian cell types. Using these systems, we identified a new MAC effector with nuclease activity that is responsible for cytotoxicity in both cell types. Our results indicate that MACs can interact with a range of host cells; and, a specific effector mediates killing of eukaryotic cells.

### Results

### MACs Kill Insect Cell Lines In Vitro

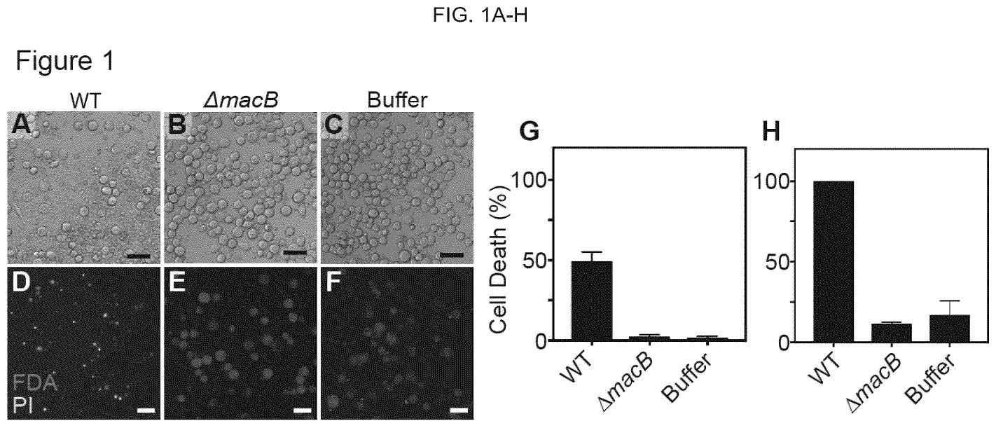

To study MACs from P. luteoviolacea and test their effect on eukaryotic cells, we focused on insect cell lines from the Fall Armyworm Spodoptera frugiperda Sf9), the closest relative to Hydroides where established cell lines are commercially available. Upon co-incubation of purified MACs, we observed the lysis of Sf9 cells within 48 hours (FIG. 1A). As a control, we included cell-free purifications from a strain lacking the MAC baseplate gene macB, which is unable to produce intact phage tail-like structures and multi-tailed arrays (Shikuma et al., 2014). When Sf9 cells were co-incubated with purifications from a macB strain or extraction buffer alone, the cells remained viable (FIGS. 1B and C). We quantified the activity of MACs against Sf9 cells by staining dead cells using a colorimetric stain, trypan blue, or by a fluorescent dual stain, fluorescein diacetate (FDA) and propidium iodide (PI), which stains live and dead cells, respectively. Using both methods, we observed cell death when cells were exposed to wild-type MACs, while death was not observed with purifications from a AmacB strain or the extraction buffer (FIG. 1D-H). Our results demonstrate that MACs are capable of targeting and killing insect cell lines.

FIG. 1A-H: MACs cause cytotoxicity in Sf9 insect cells. (A-C) Sf9 cells after 48 hours incubation with MACs from wild-type P. luteoviolacea (WT), ΔmacB mutant strain, or extraction buffer. (D-F) Live/dead staining with fluorescein diacetate (FDA) (live cells) and propidium iodide (PI) (dead cells). Scale bar is 50 μm. Quantification of cell death (%) utilizing (G) trypan blue and (H) FDA/PI live-dead stain.

### Identification of a MAC Effector Required for Killing of Insect Cells

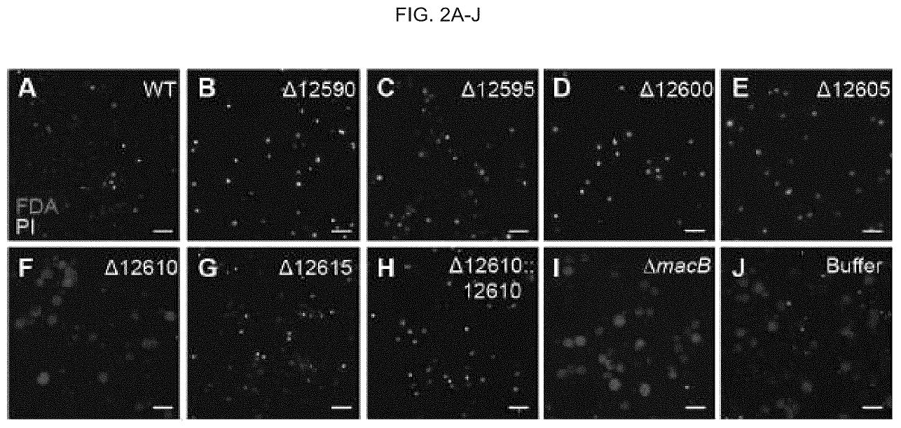

We previously showed that a locus containing six genes (JF50_12590-JF50_12615) within the P. luteoviolacea genome is required for MACs to stimulate the metamorphosis of the tubeworm Hydroides, yet a mutant lacking all 6 genes is still able to produce intact MAC structures (Shikuma et al., 2016). To determine whether MACs require this same locus for killing of insect cell lines, we tested whether P. luteoviolacea mutants lacking each of the six genes were deficient in MAC-mediated insect cell killing. Among those six genes, only a JF50_12610 mutant was unable to cause cell death upon co-incubation with insect cells (FIG. 2A-G). These results were quantified and confirmed using trypan blue or FDA/PI staining (FIGS. 2K and L). When JF50_12610 was introduced back into its native chromosomal locus, the killing effect of MACs was restored (FIG. 2H). Intriguingly, when we tested MACs from the JF50_12610 mutant against larvae from Hydroides elegans we found that this strain was capable of stimulating metamorphosis at levels comparable to that of wild-type MACs (FIG. 2M), suggesting functional structures are still present.

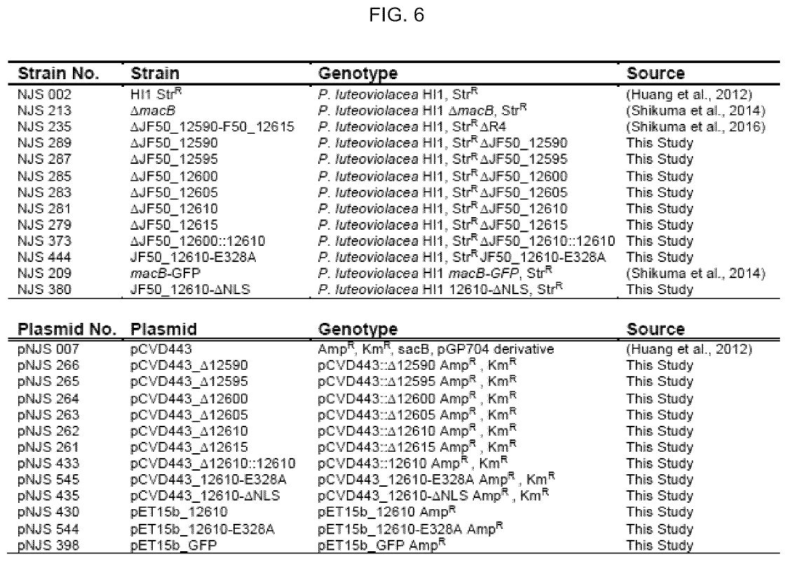

To determine whether the ΔJF50_12610 strain produces intact MAC structures, we employed electron cryo-tomography. Upon inspection, MACs from wild type and ΔJF50_12610 were indistinguishable; forming intact phage tail-like structures in both extended and contracted conformations (FIGS. 2N and O). To determine whether the ΔJF50_12610 phenotype was due to the differential production of MACs, we quantified MACs tagged with super folder GFP by fluorescent microscopy and found no difference in quantity between wild-type and ΔJF50_12610 strains grown under identical conditions (FIG. 5). Our results show that JF50_12610 is required for MACs to kill insect cell lines yet does not affect the ability of MACs to stimulate tubeworm metamorphosis or the production of functional MACs.

FIG. 2A-O. MACs require JF50_12610 to kill insect cells. (A-J) Sf9 cells after 48 h incubation with MACs from WT, ΔJF50_12590, ΔJF50_12595, ΔJF50_12600, ΔJF50_12605, ΔJF50_12610 (Δpne1), ΔJF50_12615, ΔJF50_12610 ::JF50_12610, ΔmacB strains, and extraction buffer. Scale bar is 50 μm. (K and L) Quantification of cell death (%) by trypan blue and FDA/PI live-dead staining. (M) Metamorphosis (%) of Hydroides larvae in response to MACs from WT, ΔmacB or ΔJF50_12610 strains. Electron cryo-tomography images of MACs from (N) wild type and (O) ΔJF50_12610 strains showing an ordered structure with extended and contracted tubes connected by a meshwork of tail fibers. No structural differences between WT and ΔJF50_12610 arrays were observed. Shown are projections of 1.1 nm thick slices of cryotomograms. Scale bars are 100 nm.

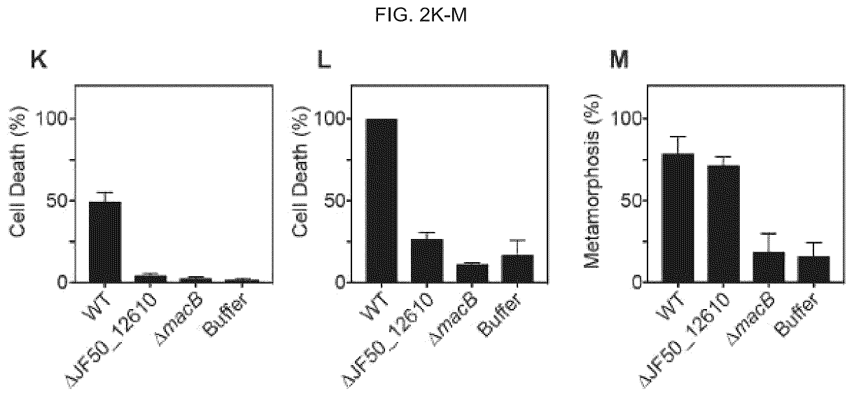

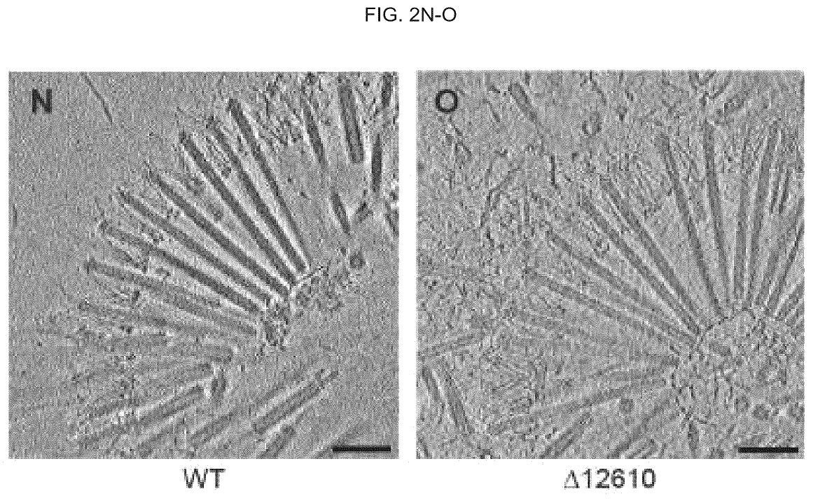

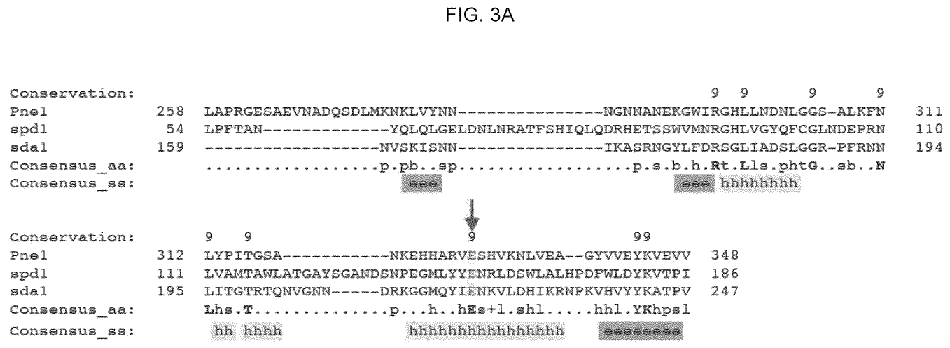

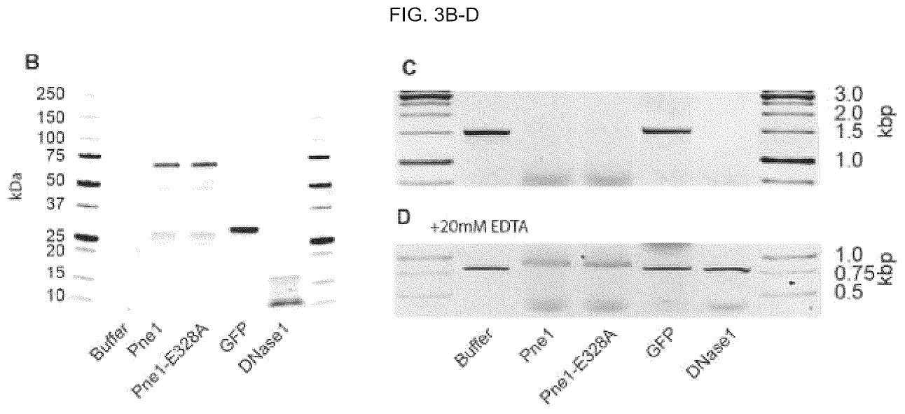

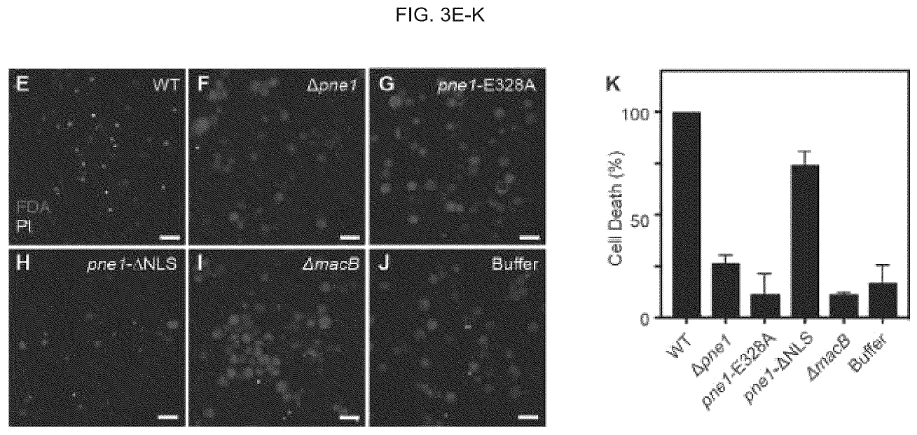

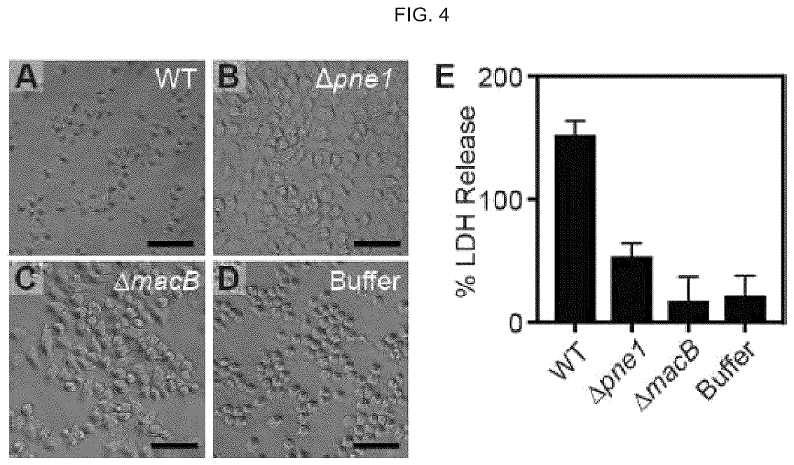

The JF50_12610 protein possesses nuclease activity in vitro and this activity is necessary for insect cell death. To determine the function of JF50_12610, we searched the 496 amino acid long protein for conserved domains and homologous proteins. We found that JF50_12610 contains a DNA/RNA non-specific nuclease domain (Pfam: PF13930). Analysis with the Phyre2 protein prediction program (Kelley et al., 2015) showed that residues 258-348 of JF50_12610 bear 20% identity to the nuclease Spd1 from Streptococcus pyogenes (Korczynska et al., 2012), and residues 267-348 bear 30% identity to the nuclease Sda1 also from S. pyogenes (Moon et al., 2016) (FIG. 3A). Through these partial alignments, we identified a conserved glutamic acid at residue 328 corresponding to Glu164 of Spd1 and Glu225 of Sda1 that coordinate water molecules hydrating the magnesium in the enzyme's active site (Korczynska et al., 2012; Moon et al., 2016). Consistent with its predicted function as a toxic effector against eukaryotic cells, JF50_12610 protein is predicted to contain a nuclear localization signal; NLStradamus program (Nguyen Ba et al., 2009), which typically targets proteins to the nucleus of eukaryotic cells. Based on the predicted function of JF50_12610 and the results below, we named this effector Pne1 for Pseudoalteromonas nuclease effector 1.

To determine whether Pne1 possesses nuclease activity, we cloned the wild type pne1 gene and a pne1-Glu328Ala mutant into an IPTG-inducible vector system with N-terminal 6xHis tag and purified both proteins by nickel affinity chromatography (FIG. 3B). When co-incubated with double-stranded DNA, Pne1 and Pne1-Glu328Ala both exhibited nuclease activity (FIG. 3C), whereas a control protein, green fluorescence protein (Gfp), cloned and purified under the same conditions, did not exhibit DNase activity. Based on similarities between Pne1 and magnesium-dependent homologous proteins Spd1 and Sda1, we tested the DNase activity of Pne1 protein in the presence of the divalent cation chelator, EDTA. Surprisingly, Pne1 and the Pne1-Glu328Ala proteins were still functional in the presence of EDTA (FIG. 3D).

To test whether Pne1 requires its nuclear localization signal or the conserved Glu328 for killing insect cells, we created P. luteoviolacea mutants lacking the predicted nuclear localization signal (residues 19-52) pne1-ANLS or with the pne1-Glu328Ala point mutation in their native chromosomal loci. Upon exposure to insect cells, MACs from the pne1-ANLS strain partially abolished the killing effect and MACs from the pne1-Glu328Ala strain were unable to kill insect cells when compared to wild-type MACs (FIG. 3G, H, K). Our results show that Pne1 possesses nuclease activity in vitro and its nuclear localization signal may be partially responsible for the killing effect. While the Glu328Ala is not required for nuclease activity in vitro, this residue is necessary for MACs to kill insect cells.

FIG. 3A-K. JF50_12610 (Pne1) contains a functional nuclease domain that is required for insect cell killing. (A) Protein alignment of Pne1 (JF50_12610), Spd1 and Sda1. Numbers indicate amino acid residues of each protein. Conserved amino acid residues indicated in bold. Consensus secondary structure (ss) alpha-helix (h, yellow) and beta-strand (e, blue). A conserved glutamic acid 328 is indicated by an arrow and highlighted in magenta. (B) SDS Page gel of purified wild type Pne1, Pne1-Glu328Ala, Gfp, and DNase1. Representative 1% agarose gel of DNA co-incubated with Pne1, Pne1-Glu328Ala and Gfp at 37° C. for 2 hour (PCR product size, 1.5 Kbp) in the absence (C) or presence (D) of 20mM EDTA. Live/Dead images of Sf9 insect cells after 48 hours incubation with MACs from (E) WT, (F) Δpne1, (G) pne1-Glu328A, (H) pne1-ΔNLS, (I) AmacB, and (J) MAC extraction buffer. Scale bar is 50 μm. (K) Quantification of cell death (%) by FDA/PI live-dead stain.

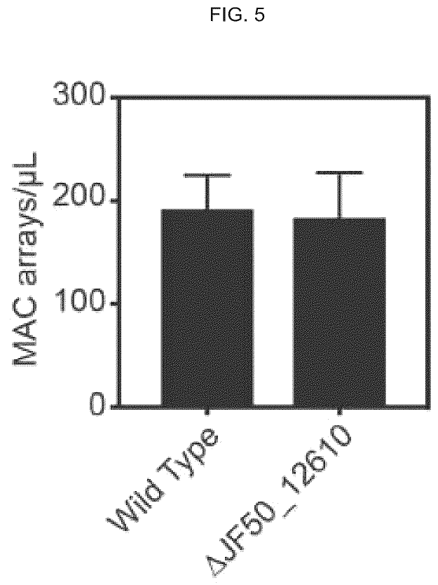

MACs possess a broad host range, killing J774A.1 murine macrophage cell line in vitro. To determine whether MACs are capable of targeting a broader range of eukaryotic cells, we tested the ability of MACs to kill mammalian cells. We chose commonly used murine macrophage cell line J774A.1 as these immune cells often encounter microbial pathogens and their effectors. Upon exposure of J774A.1 cells to wild-type MACs, we observed cell death within 24 hours (FIG. 4A). In contrast, MACs from a Δpne1 and ΔmacB, or buffer alone did not exhibit cell killing (Figure B-D). Quantification of cytotoxicity by lactate dehydrogenase (LDH) release assays confirmed the observed killing upon exposure to wild-type MACs and lack of killing upon exposure to MACs from the Δpne1 (FIG. 4E). Taken together, our results show that MACs are capable of targeting and killing mammalian cells and the cell killing phenotype is dependent on the presence of Pne1.

FIG. 4A-E. MACs kill J774A.1 murine macrophages and killing is dependent on JF50_12610. (A-D) J774A.1 cells after incubation with MACs from WT, ΔJF50_12610, ΔmacB or extraction buffer. Scale bar is 50 μm. (E) Cell death was quantified by Lactate Dehydrogenase (LDH) release assay at 24 hours.

### Discussion

In this work, we establish an in vitro interaction model between an eCIS and two eukaryotic cell lines. With this system, we determined that the bacterial protein, Pne1, is a novel eCIS effector and possesses nuclease activity. To our knowledge, our work is the first to identify a CIS with broad eukaryotic host range and identify the first eukaryotic-targeting CIS effector with nuclease activity.

Here, we show Pne1-dependent cell death of insect and mammalian cell lines, yet we have also previously observed that MACs stimulate metamorphosis in the tubeworm Hydroides (Shikuma et al., 2014). While it is unclear why MACs possess an effector-mediated killing of eukaryotic cells, some symbiotic bacteria have been shown to use CIS to modulate their host range. For example, the nitrogen-fixing plant symbiont Rhizobium leguminosarum limits its host range to plants in the clover family by secreting proteins through a T6SS, while mutation of the imp gene cluster (encoding components of the T6SS) allowed the bacterium to form functional root nodules on pea plants, normally outside of its host range (Bladergroen et al., 2003). In the environment, Pseudoalteromonas species are found in association with many marine invertebrates (Holmstrom and Kjelleberg, 1999), and might utilize MACs and Pne1 to antagonize specific eukaryotes, like bacterivorous ciliates, while also stimulating the metamorphosis of other eukaryotes like Hydroides. While several CIS nuclease effectors have been identified targeting bacterial cells, including Tde1 from the soil bacterium Agrobacterium tumefaciens (Ma et al., 2014), RhsA from D. dandantii (Koskiniemi et al., 2013), and RhsP from Vibrio parahaemolyticus (Jiang et al., 2018), Pne1 is the first CIS nuclease effector to our knowledge that targets eukaryotic organisms. Interestingly, the eukaryotic NLS at the N-terminus of Pne1 had a partial effect on its ability to kill insect cells, further implying its evolved role in targeting eukaryotic hosts.

The host range of eukaryotic-targeting eCISs are currently poorly understood. Intriguingly, work on related eCIS show that many of them target eukaryotic organisms from diverse lineages (e.g. Grass Grubs, Wax moth, Wasps, and Amoeba) (Böck et al., 2017; Hurst et al., 2007; Penz et al., 2010; Yang et al., 2006). While the ability of related eCIS, the Anti-feeding prophage (Afp) and Photorhabdus Virulence Cassettes (PVC), to target eukaryotic cells has been attributed to tail fibers that resemble receptors from adenovirus (Hurst et al., 2007; Yang et al., 2006), we detected no similar homology in MAC structures. We report here that MACs are capable of targeting multiple in vitro cell lines from insect and mammalian lineages. By further studying how eCIS, like MACs, have evolved from bacteriophage origins to target eukaryotic cells, we can begin to determine the underlying mechanisms associated with these diverse interactions.

In addition to expanding our basic understanding of CIS, our work opens the potential for using eCIS for biotechnology purposes. eCIS that target bacterial pathogens are already under development as narrow host-range antimicrobial agents (Scholl, 2017), for example against the gastrointestinal pathogen, Clostridium difficile (Gebhart et al., 2015).

As syringe-like structures that deliver proteinaceous cargo to eukaryotic cells, provided herein are MACs as delivery systems for biotechnology applications. In alternative embodiments, a genetically-modified CIS is provided to deliver peptides of interest to specific eukaryotic cell types. Data using the in vitro system described in this work demonstrates the efficacy of embodiments as described herein.

### Materials and Methods

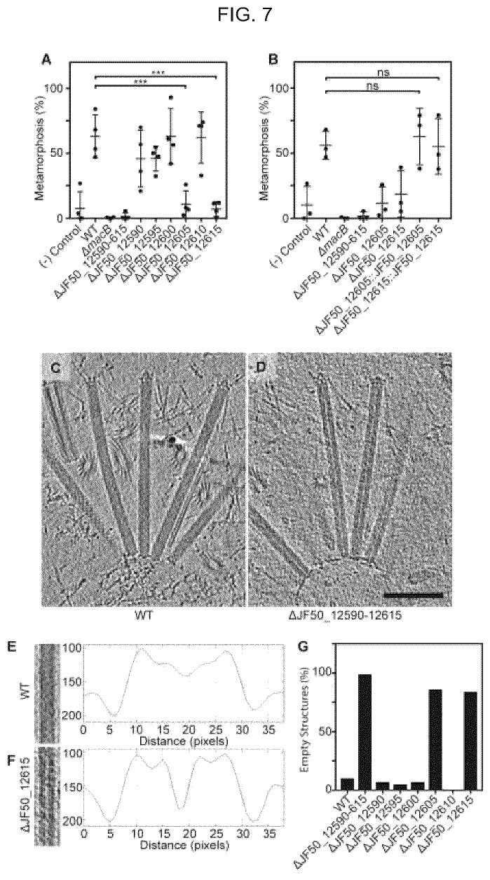

All the strains used throughout the experiments reported are shown in FIG. 6, and the primers used throughout the experiments of Example 1 are listed below. All deletion and fusion strains were created according to previously published protocols (Shikuma et al., 2014, 2016). Plasmid insert sequences were verified by DNA sequencing. Deletion and insert strains were confirmed by PCR. All E. coli strains were grown in Luria-Bertani (LB) media at 37° C.shaking at 200 revolutions per minute (RPM). All P. luteoviolacea cultures were grown in seawater tryptone (SWT) media (35.9 g/L Instant Ocean™, 2.5g/L tryptone, 1.5 g/L yeast extract, 1.5 ml/L glycerol) at 25° C. shaking at 200 RPM. Media that containing antibiotics were at a concentration of 100 mg/mL unless otherwise stated.

### MACs Extractions

MACs are produced by the marine bacteria Pseudoalteromonas luteoviolacea as described previously (Shikuma et al., 2014). Briefly, cells were struck out from frozen stock on SWT agar and grown at 25° C. or room temperature for 1-2 days. Cells were inoculated into 5 mL SWT broth and grown for 24 hours, 25° C. at 200 rpm. Cultures were inoculated 0.5 mL into 50 mL SWT in a 250 mL flask and grown for 16 hours, 25° C. at 200 rpm. Cultures were transferred to a 50 ml conical centrifuge tubes and centrifuged at 4000 g for 20 minutes at 4° C. Cultures were kept on ice after this step. Subsequently, the supernatant was discarded and the pellet was resuspended in 5 mL of cold extraction buffer (20 mM Tris Base, 1M NaCl, 1 L of deionized water, adjust the pH to 7.5 with HCl). The resuspension mixture was then transferred to a 15 mL centrifuge tube and placed in the centrifuge for another 20 minutes at 4000 g at 4° C. This time, the supernatant was transferred to another 15 ml tube and spun down one last time with the same above conditions. After this final spin, the supernatant (now called MAC extract) was carefully poured to another clean 15 mL tube and kept at 4° C. until it was ready to be used for cell infections.

### Macrophage Cell Culture

To test the effect of P. luteoviolacea MACs on eukaryotic cells, we used two cell lines; murine macrophages J774A.1, and Sf9 insect cells. Macrophage cells were cultured in DMEM (Dulbecco's Modified Eagle's Medium, Gibco #10566-016), which was prepared with the addition of 10% of heat-inactivated fetal bovine serum (FBS) and 1% sodium pyruvate. Frozen cell line stocks were taken from nitrogen tank and thawed quickly at 37° C. in a water bath. Thawed cells (approximately 1 mL) were added to a 50 mL conical centrifuge tube containing 5 mL of pre-warmed DMEM. Cells were then centrifuged at 500 g at room temperature for 4 minutes. The supernatant was carefully discarded, and cells were resuspended in 7.5 mL of pre-warmed DMEM. This step assured complete removal of DMSO from frozen cell stocks. Resuspended cells were transferred from the 50 ml conical centrifuge tube into a 25 cm² tissue culture flask and incubated at 37° C. with 5% CO₂. Cell lines were maintained by passaging every 2-3 days using routine cell culture techniques.

### Macrophage Cell Co-Incubation with MACs and LDH Assay

Prior to infecting with MACs, J774A.1 cells were passaged two to five times and seeded into 24-well tissue culture plates at a density of 4×10\hat{ }5 cell/mL the night before the infection. MACs that were extracted no more than a week prior to cell infections were added to each well of the 24-well plates with cells at a 1:50 ratio (10 uL of the extracts into 500 ul of cells). Infected cells were incubated at 37° C. with 5% CO2. Culture supernatants were collected at between 1 and 24 hours post-infection. At each time point, the plate was first spun down at 400 g for 2 minutes, then 100 uL of the supernatant was transferred into a 96-well plate in duplicate. For t=0 time point, remaining supernatants were removed and cells were lysed with PBS+1% Triton X™ 100 μl of the cell lysates was also transferred to 96-well plates. Microscopic images were taken of each well to visualize and record the cells viability, using a phase-contrast microscopy.

To assess cell lysis, we quantified the release of lactate dehydrogenase (LDH) using Takara LDH Cytotoxicity Detection Kit™ (#MK401). The LDH reaction mix was made per manufacturer's instructions and kept away from the light. Cell supernatants were diluted 1:10 in PBS and diluted supernatants were mixed 1:1 with the LDH reaction mix. After the addition of the reaction mix, plates were incubated for 30 minutes at room temperature away from the light and absorbance was measured at 492 nm using a plate reader. Total LDH level at t=0 was calculated by adding LDH levels of t=0 supernatants and lysates. LDH release 24 hours post-infection was calculated as a fraction of total LDH available at the time of infection (t=0).

### Insect Cell Culture

To test the effect of P. luteoviolacea MACs interaction with eukaryotic insect cell lines, we used Sf9 cells (Novagen #71104-3). Cells were cultured in ESF 921 Insect Cell Culture Medium (Expression systems #96-001-01). Frozen cell line stocks were taken from −80° C. freezer and thawed quickly at 27° C. in a water bath. Thawed cells (˜1 mL) were added to a 125 mL flask containing 10 mL of room temperature ESF 921. Cells were then placed in a 27° C. incubator shaker, shaking at 130 rpm, in the dark. Cell lines were maintained by passaging every 2-3 days using routine cell culture techniques.

### Insect Cell Co-Incubation with MACs

Prior to infecting with MACs, Sf9 cells were passaged five to twenty times and seeded into 24-well tissue culture plates at a density of 4×10\hat{ }5 cell/mL a few hours before infection. MACs that were extracted no more than a week prior to cell infections were added to each well of the 24-well plates with cells at a 1:50 ratio (10 uL of the extracts into 500 ul of cells). Infected cells were incubated at 27° C., adherently. At 48 hours, trypan blue or PI/FDA was added to each well of the 24-well infection plates and microscopic images were taken of each well to visualize and quantify cell viability.

### Protein Purification

The JF50_12610 wild type, JF50_12610-Glu328Ala and Green Fluorescence Protein (GFP) were cloned into a pET15b vector with N-terminal 6xHis tag. The protein was overexpressed in 500 mL of LB medium using 0.5 mM IPTG, followed by a centrifugation step of 4000 g for 20 min at 4° C. The cell pellet was resuspended using a native lysis buffer (0.5 M NaCl, 50 mM Tris-HCl, 10% glycerol, 20 mM imidazole, 0.2% TritonXTM, pH 8). The resuspended cell lysate was then submitted to a cell press, which was followed by sonication (30 seconds sonication, repeated twice). Following cell sonication, the supernatant was purified by a FPLC (AKTA start by GE), where protein fractions were collected. Collected protein fractions were then quantified using a Thermo-Fisher™ pre-diluted protein standards kit (catalog #23208), and subsequently read on a Biotek™ plate reader (catalog #49984). Proteins were then normalized to equal concentrations prior to DNAse assay.

### Nuclease Assay

In order to test the bioinformatically predicted nuclease activity found in JF50_12610, a DNase assay was developed. The wild type JF50_12610, JF50_12610-E328A and Green Fluorescence Protein (GFP) were purified simultaneously and identically prior to assay. After protein purification, protein concentrations were normalized to 0.5 ug/uL, including a positive control commercially available DNase I (BioBasic™ #DD0099-1). All normalized proteins were then incubated for 1 hour at 37° C. water bath with a PCR DNA fragment of known size and concentration and NEB cutsmart buffer (New England Biosciences #B72045). A reaction total volume of 10 uL consisted of 6 uL of protein, 3 uL of DNA, and 1 L of buffer. After 1-hour incubation, all reactions and their replicates were resolved in an 1% agarose gel stained with EtBr in 1XTBE (Tris-Borate EDTA, BioBasic #A00265) at 110 volts for about 45 minutes. Gel products were then visualized in a BioRad gel imaging machine (Gel Doc™ EZ System #1708270) appropriately.

### MAC Quantification

The quantification of MACs followed the same protocols and methods from with the following mentioned modifications(Thurber, Rebecca, Haynes, Matthew, Breitbart, Mya, Wegley, Linda, Rohwer, 2009). MACs do not contain nucleic acids, and therefore no SYBR staining occurred. We used a 0.22 um anodisc to filter clusters using the same apparatus set forth by Rower and group. First, the anodisc was placed on the vacuum stage with its shiny side facing up. Then, the glass column and clamp were secured on top of it and 2 mL of 1×PBS was added to it to run thru the column. Once the PBS was filtered and the anodisc apparatus was successfully placed, 100 uL of extracted MACs was added to 900 uL of 1×PBS and allowed to run thru the filter. Once all the liquid passed through the anodisc, the clamp and column were taken out before shutting off the vacuum to assure that the anodisc stayed on the stage. The anodisc was placed on a kimwipe to allow it to dry properly for a few minutes. Once dried, a slide was made using 10 uL of the microscope mount (10% ascorbic acid 1×PBS with 50% glycerol, filter sterilize mount with 0.02 um filter) “sandwiching” the anodisc. Using the ZEISS microscope set up to a green fluorescence protein emission channel, we were able to quantify wild type MACs that were tagged with sf-GFP. Calculation extrapolations were made based on column diameter, field of view and volume placed on the disc.

### Electron Cryo-Tomography of MAC Arrays

For ECT imaging of MAC arrays, P. luteoviolacea WT and ΔJF50_12610 were cultured for 5-6 h at 30° C. and subsequently centrifuged for 30 min at about 7000 g. The pellet was resuspended in 5 mL of extraction buffer and centrifuged for 30 min at 4000 g to separate intact cells from MAC arrays. The supernatant was carefully transferred into a new tube and centrifuged for 30 min at 7000 g. The pellet was resuspended in about 50 μL of extraction buffer and mixed with Protein A-conjugated 10 -nm colloidal gold (Cytodiagnostics Inc.) before plunge freezing. Plunge freezing and ECT imaging were performed according to Weiss et al. (Weiss et al., 2017). 4 μL of sample was applied to glow-discharged EM grids (R2/1 copper, Quantifoil), blotted twice from the back for 3.5 s and vitrified in liquid ethane-propane using a Vitrobot Mark IV (ThermoFisher). ECT data were collected on a Titan Krios (ThermoFisher) transmission electron microscope equipped with a Quantum LS imaging filter (Gatan, slit with 20 eV) and K2 Summit direct electron detector (Gatan). Tilt series were acquired with the software SerialEM (Mastronarde, 2005) using a bidirectional tilt scheme. The angular range was −60° to +60° and the angular increment was 2°. The total electron dose was between 60-100 electrons per Å² and the pixel size at specimen level was 2.72 Å. Images were recorded in focus with a Volta phaseplate (ThermoFisher) for WT and without phaseplate at 5 μm under-focus for ΔJF50_12610. Tilt series were aligned using gold fiducials and three-dimensional reconstructions were calculated by weighted back projection using IMOD (Mastronarde, 1997). Visualization was done in IMOD. Contrast enhancement of the ΔJF50_12610 tomogram was done using the tom_deconv deconvolution filter (https://github.com/dtegunov/tom_deconv).

FIG. 5: A P. luteoviolacea JF50_12610 mutant produces the same quantity of intact MACs as wild type. Quantification of MAC production between wild type and ΔJF50_12610 strains. Both strains were tagged with a super folder-GFP on the baseplate as described previously (Shikuma et al., 2014). The number of MAC arrays were quantified between wild type and ΔJF50_12610 mutant using a hemocytometer and fluorescence microscopy.

### List of Strains Produced

see FIG. 6.

### Primers Used.

| Primer | Sequence |
|---|---|
| macB_dA | GGTCGACGGATCCCAAGCTTCTTCTAGAGGTACCGCATGCAA |
| | CCCAGACACTGAGGTGCT (SEQ ID NO: 3) |
| macB_dB | TTTCCATTTTCCAATCCCTTCGCCAGAGATAAGTGATTGACTA |
| | CGA (SEQ ID NO: 4) |
| macB_dC | TCGTAGTCAATCACTTATCTCTGGCGAAGGGATTGGAAAATG |
| | GAAA (SEQ ID NO: 5) |
| macB_dD | ACACAACGTGAATTCAAAGGGAGAGCTCGATATCGCATGCCA |
| | TAACCTGGCTGAGCACCT (SEQ ID NO: 6) |
| 1556_dA | TGATGGGTTAAAAAGGATCGATCCTCTAGATTGGAGCAATAA |
| | ACGGGTTC (SEQ ID NO: 7) |
| 1556_dB | GTTCATAATTAAACTGCGATCGCAGCCATAAGGCCTCCTTGAT |
| | A (SEQ ID NO: 8) |
| 1556_dC | TATCAAGGAGGCCTTATGGCTGCGATCGCAGTTTAATTATGA |
| | AC (SEQ ID NO: 9) |
| 1556_dD | TTTTGAGACACAACGTGAATTCAAAGGGAGAGCTCCGCTTTG |
| | GGTACTGGCTTTA (SEQ ID NO: 10) |
| 1556_intF | CCGAGCAAACGTTATCACAA (SEQ ID NO: 11) |
| 1556_intR | TCAGCGCTCTCATTATGTGC (SEQ ID NO: 12) |
| 1555_dA | TGATGGGTTAAAAAGGATCGATCCTCTAGACCGAGCAAACGT |
| | TATCACAA (SEQ ID NO: 13) |
| 1555_dB | CCTTGCATGAGGTTAAGAAAGTTTGACGTACCCTTCAGCCAT |
| | ATT (SEQ ID NO: 14) |
| 1555_dC | AATATGGCTGAAGGGTACGTCAAACTTTCTTAACCTCATGCA |
| | AGG (SEQ ID NO: 15) |
| 1555_dD | TTTTGAGACACAACGTGAATTCAAAGGGAGAGCTCGATGCGG |
| | TAACGGTTGTTCT (SEQ ID NO: 16) |
| 1555_intF | AGCGATTGATGCTGAACAAA (SEQ ID NO: 17) |
| 1555_intR | ACCATCGCATAACCCGTAAC (SEQ ID NO: 18) |
| 1554_dA | TGATGGGTTAAAAAGGATCGATCCTCTAGATACGCCGTCCAG |
| | TTAGGACT (SEQ ID NO: 19) |
| 1554_dB | GTTTGTTAACGTCACGGCAGCTGCATTGCCATTTAAACTCC |
| | (SEQ ID NO: 20) |
| 1554_dC | GGAGTTTAAATGGCAATGCAGCTGCCGTGACGTTAACAAAC |
| | (SEQ ID NO: 21) |
| 1554_dD | TTTTGAGACACAACGTGAATTCAAAGGGAGAGCTCATTGATT |
| | GGAAGCGCGATAG (SEQ ID NO: 22) |
| 1554_intF | TTTATGAGGCACCAACGACA (SEQ ID NO: 23) |
| 1554_intR | GCCTGTGCCGTTTTATCTGT (SEQ ID NO: 24) |
| 1553_dA | TGATGGGTTAAAAAGGATCGATCCTCTAGAGGCGATCAGTGG |
| | AGTGAAGT (SEQ ID NO: 25) |
| 1553_dB | AATACTTCTTGCTCAGCCCCGCGTGCTTCTTCTGTCATGT |
| 1553_dC | ACATGACAGAAGAAGCACGCGGGGCTGAGCAAGAAGTATT |
| | (SEQ ID NO: 26) |
| 1553_dD | TTTTGAGACACAACGTGAATTCAAAGGGAGAGCTCTCAGAAC |
| | CAGCAGTCTCACG (SEQ ID NO: 27) |
| 1553_intF | CGGGCCTAGAAATCACTCAA (SEQ ID NO: 28) |
| 1553_intR | TCGACGTCAAATCAGTCGAG (SEQ ID NO: 29) |
| 1552_dA | TGATGGGTTAAAAAGGATCGATCCTCTAGAGAGAGCAAGAA |
| | GTGGCGAGT (SEQ ID NO: 30) |
| 1552_dB | TAGCCTTTTAGTGCCGCTTTTGAGGCGTCCATATCTGACA |
| | (SEQ ID NO: 31) |
| 1552_dC | TGTCAGATATGGACGCCTCAAAAGCGGCACTAAAAGGCTA |
| | (SEQ ID NO: 32) |
| 1552_dD | TTTTGAGACACAACGTGAATTCAAAGGGAGAGCTCTGCTGAC |
| | CAAGCAGATTGAC (SEQ ID NO: 33) |
| 1552 intF | GGGCAATTGTTGTGGATTTT (SEQ ID NO: 34) |
| 1552 intR | TGATCCCAAACCACTTGTGA (SEQ ID NO: 35) |
| 1551_dA | TGATGGGTTAAAAAGGATCGATCCTCTAGAGACTGCTGGTTC |
| | TGATTCGAT (SEQ ID NO: 36) |
| 1551_dB | AACAGATCATTACATTAAAATGAGCCTCTGTTCTTGTTGTTGC |
| | ATTTCA (SEQ ID NO: 37) |
| 1551_dC | TGAAATGCAACAACAAGAACAGAGGCTCATTTTAATGTAATG |
| | ATCTGTT (SEQ ID NO: 38) |
| 1551_dD | TTTTGAGACACAACGTGAATTCAAAGGGAGAGCTCCTTCTCC |
| | ATTTTCGCCTTTG (SEQ ID NO: 39) |
| 1551_intF | CGTTTTCAGTGACCATCACG (SEQ ID NO: 40) |
| 1551_intR | CGGTGGGCAAAAAGGTATAA (SEQ ID NO: 41) |
| pET15b_12610_ | CCTGGTGCCGCGCGGCAGCCATATGATGATGTCAGATATGGA |
| F1 | CGC (SEQ ID NO: 42) |
| pET15b_12610_ | TCGGGCTTTGTTAGCAGCCGGATCCTTAGCCTTTTAGTGCCGC |
| R1 | TT (SEQ ID NO: 43) |
| 12610-ΔNLS_B | ATACTCATATCTGAGTTTTGTTGCTTCTGCTCTTCGCTAT |
| 12610-ΔNLS_C | ATAGCGAAGAGCAGAAGCAACAAAACTCAGATATGAGTAT |
| | (SEQ ID NO: 44) |
| JF50_12610_ | CCTGAAGGGTCGTTTTCAGT (SEQ ID NO: 45) |
| SeqF1 | |
| JF50_12610_ | GCTCGAGTTGCGTCTCATGTA (SEQ ID NO: 46) |
| E328A_F | |
| JF50_12610_ | TACATGAGACGCAACTCGAGC (SEQ ID NO: 47) |
| E328A_R | |

### Example 2: Exemplary Methods and Compositions: A Contractile Injection System Stimulates Tubeworm Metamorphosis by Translocating a Proteinaceous Effector

This example provides exemplary methods for making compositions and bacterial cells as provided herein.

Many bottom-dwelling marine animals identify a suitable location for adult life by first producing swimming larvae that recognize cues from surface-bound bacteria to settle and undergo metamorphosis. Although this microbe-animal interaction is critical for the life cycle of diverse marine animals, what type of biochemical cue is recognized by the animal has been a mystery. For larvae of the tubeworm, Hydroides elegans, metamorphosis is induced by an extracellular syringe-like Contractile Injection System (CIS) produced by the bacterium Pseudoalteromonas luteoviolacea. In this work, we observe by electron cryotomography a proteinaceous effector within the rigid inner tube lumen of a CIS. This protein, termed Mif1, is sufficient for triggering metamorphosis when electroporated into tubeworm larvae. Our results demonstrate that a proteinaceous bacterial factor is responsible for stimulating animal metamorphosis, supporting the hypothesis that a CIS proteinaceous effector delivery mechanism may orchestrate microbe-animal interactions in diverse contexts.

In this work, we show that a previously identified genomic region in P. luteoviolacea encodes for a bacterial protein that fills the inner tube lumen of the MAC structure and is necessary for inducing the metamorphosis of Hydroides larvae. To our knowledge, our results identify the first proteinaceous effector stimulating animal metamorphosis and provide the first direct visualization of an effector within inner tube lumen of a syringe-like CIS.

### Results

Two Bacterial Genes are Responsible for Densities Within the Inner Tube Lumen of MACs and Correspond with Metamorphosis-Inducing Ability.

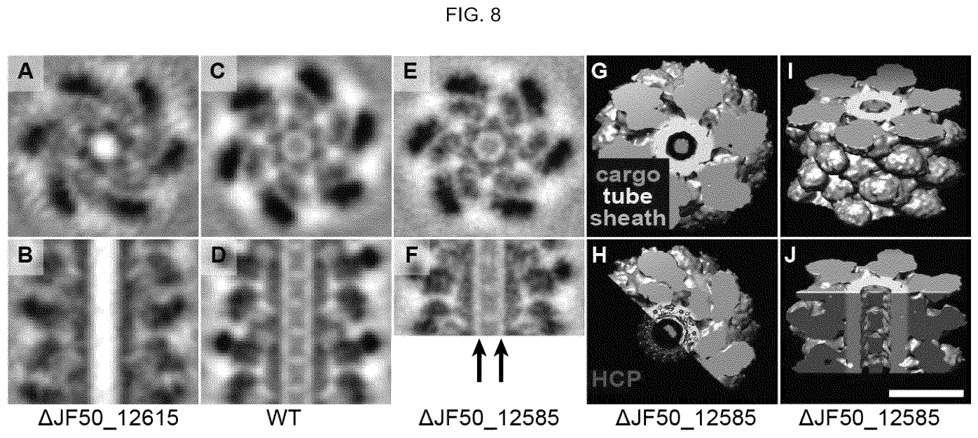

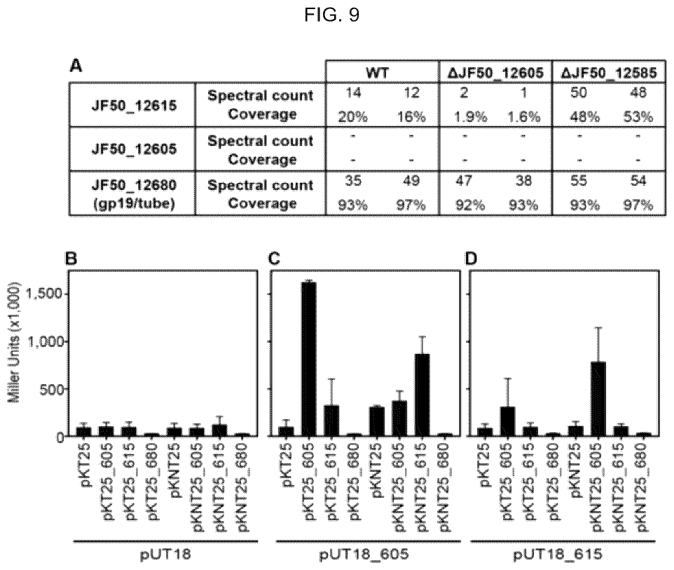

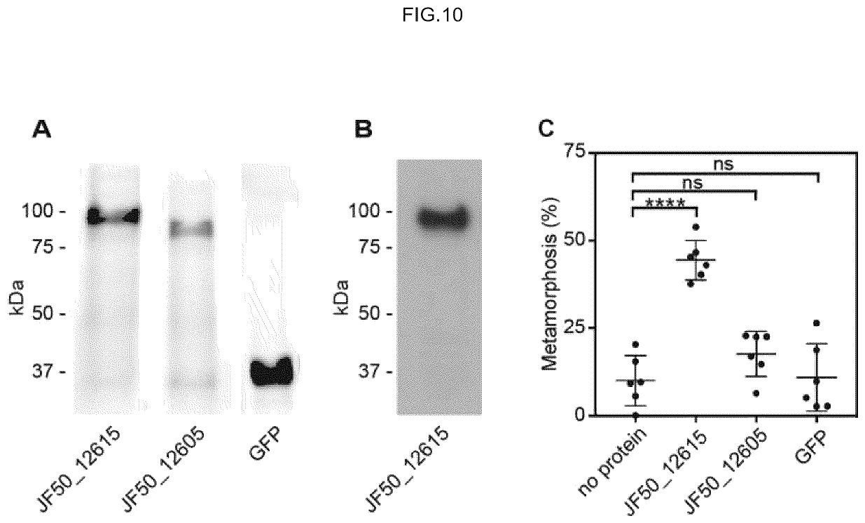

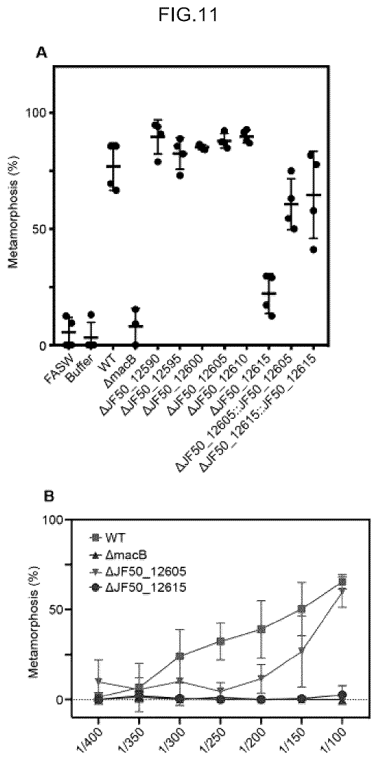

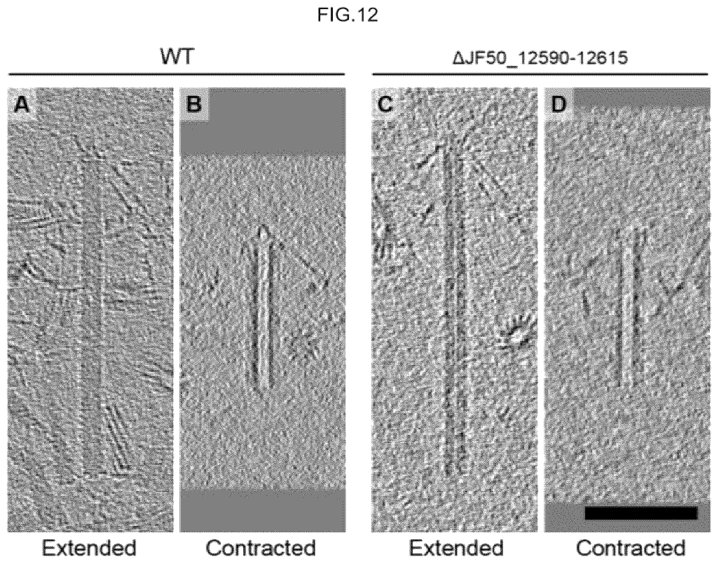

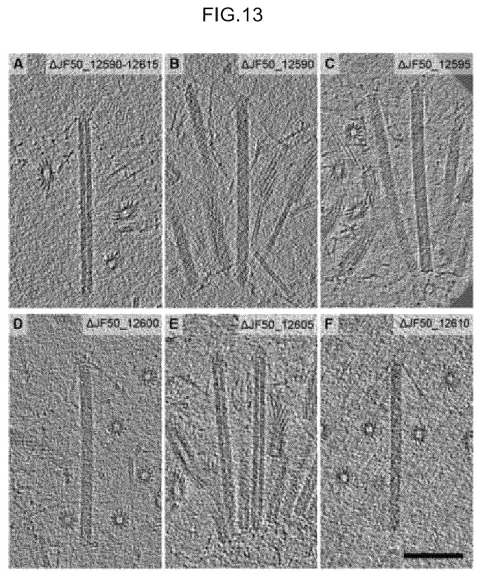

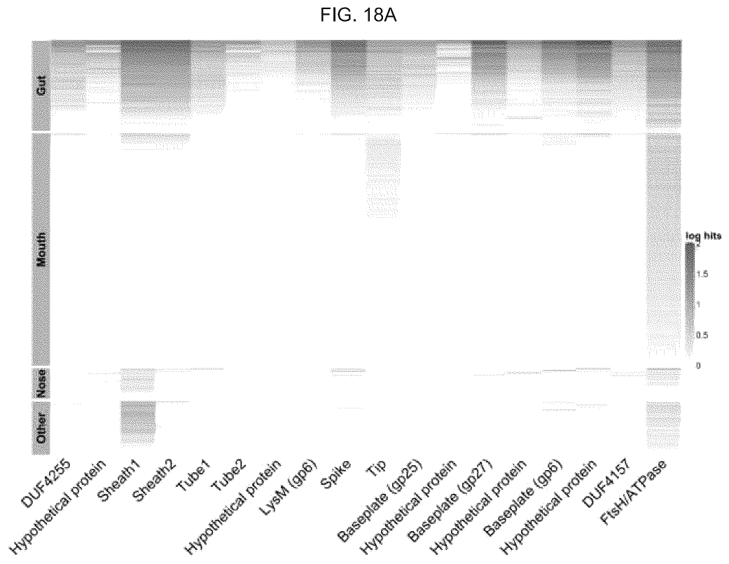

We previously identified a genomic locus in P. luteoviolacea encoding 6 genes (gene numbers JF50_12590, JF50_12595, JF50_12600, JF50_12605, JF50_12610 and JF50_12615) that are essential for inducing the larvae of Hydroides to undergo metamorphosis (Shikuma et al., 2016). When biofilms of strains with in-frame deletions of each of the six genes were analyzed for their ability to induce Hydroides metamorphosis, the ΔJF50_12605 and ΔJF50_12615 mutants exhibited a reduced ability to induce metamorphosis (less than 20%, FIG. 7A), while mutation of the other four genes had no observable effect. When JF50_12605 and JF50_12615 were replaced back into their native chromosomal loci, metamorphosis induction was restored (FIG. 7B). We confirmed the effect of MACs on Hydroides metamorphosis by producing cell-free MACs preparations. While larvae exposed to MACs from a ΔJF50_12615 mutant did not induce metamorphosis (even at high concentrations), MACs from a ΔJF50_12605 mutant induced metamorphosis when added at higher doses (Fig. S1A-B, or FIG. 11A and FIG. 11B). Our results suggest that JF50_12615 is essential, while JF50_12605 contributes but is dispensable, for the ability of MACs to induce metamorphosis.

FIG. 7A-G. Two Bacterial Genes are Important for Inducing Hydroides Metamorphosis and Produce MACs Lacking Electron Density within Their Inner Tubes:

(A) Metamorphosis (%) of Hydroides larvae in response to biofilms of P. luteoviolacea lacking JF50_12590, JF50_12595, JF50_12600, JF50_12605, JF50_12610 or JF50_12615 genes. Deletion of JF50_12605 and JF50_12615 showed a significant loss in the ability to induce metamorphosis when compared to wild type.

(B) Restoration of JF50_12605 and JF50_12615 into their native chromosomal loci restores function. Graphs in A and B show an average of biological replicates, where each point represents one biological replicate. *p-value ≤0.05, ns=not significant.

(C) Representative images of the filled tube phenotype from wild type cells and

(D) empty tube phenotype from gene deletion mutants. Scale bar, 100 nm.

(E/F) Density plots of representative sections from C and D. The empty tube phenotype has a higher intensity corresponding to the empty lumen.

(G) Fraction of empty structures for the JF50_12590-JF50_12615 region and each gene deletion.

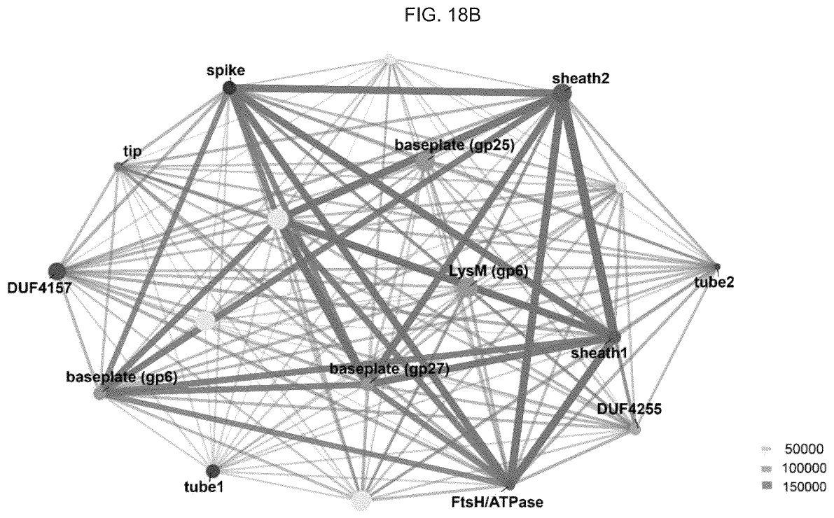

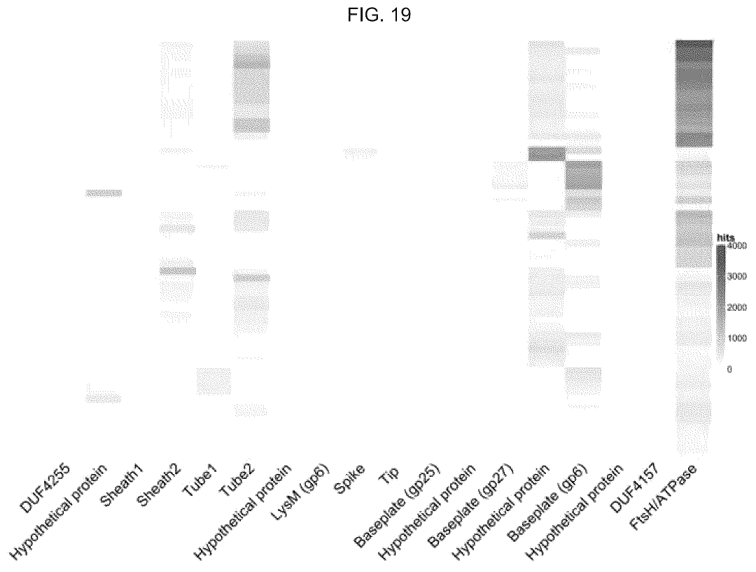

To search for structural differences between MACs from wild type P. luteoviolacea and the specific gene mutants, we employed electron cryotomography (ECT). Deletion of the full JF50_12590-JF50_12615 locus (Fig. S2, or FIG. 12), or each of the six genes individually (Fig. S3, or FIG. 13), did not impair the formation of ordered arrays of MACs, featuring both extended and contracted conformations. Upon detailed analyses, we observed that extended MACs from both AJF50_12605 and ΔJF50_12615 strains exhibited a central lumen with very low density. By contrast, MACs from wildtype and the other deletion mutants possessed a density distribution that was homogeneous and a lumen was not discernable (FIG. 7C-F and Fig. S3, or FIG. 13). We refer to these structural phenotypes as “empty” and “filled” respectively. Strikingly, quantitative analyses showed that the empty phenotype in ΔJF50_12605 and ΔJF50_12615 MACs correlated with the inability to induce metamorphosis (FIGS. 7A and G).

### The Density Within the MAC Tube Lumen Represents a Cargo Protein.

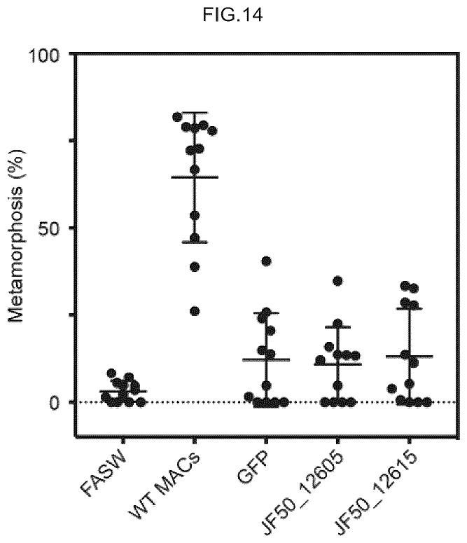

To investigate whether the difference in density observed in filled and empty MACs correspond to the presence or absence of a CIS inner tube (gp19) or rather to the presence or absence of a potential cargo within the tube lumen, we averaged sub-volumes of the sheath-tube complex. Initial averages of both wildtype and ΔJF50-12615 extended MACs revealed densities that might correspond to the sheath and the inner tube. While the approximately 4 nm wide inner tube lumen of MACs from the ΔJF50_12615 lacked any discernable density, the wild-type structure exhibited repeating packets of densities inside the tube (FIG. 8A-D). In order to improve the quality of the filled tube-sheath complex, we generated another average using a mutant in which a tail pin-like gene (JF50_12585) was deleted. We found that this mutant consisted of an increased fraction of extended MACs, providing a higher number of subvolumes for averaging. By docking a homology model of the gp19 protein into the average, we identified densities corresponding to the tube with high confidence (FIG. 8G-J). The densities in the center were clearly not accounted for by the gp 19 structure, suggesting that they represent a potential cargo within the tube lumen. In general, the densities in the tube lumen reinforced less strongly, compared to the gp 19 tube and sheath. This could have different explanations: 1) during averaging, the alignment of the cargo was affected by the strong densities from sheath-tube, 2) the cargo was not structured, or 3) the cargo was present at a sub-stoichiometric amount compared to gp 19 subunits or gp19-rings. It is noteworthy, however, that the potential cargo and tube were observed to be generally separated by a low-density region.

FIG. 8A-J. The lumen of MACs from a ΔJF50-12615 mutant lacks electron density. (A-D): Cross sectional (A and C) and longitudinal (B and D) slices through initial subtomogram averages (pixel size of 4.28 Å) displayed a clear density difference of the lumen of the inner tube from the wild type structures when compared to the JF50_12615 mutant structure. (E-F) An improved average (pixel size of 2.72 Å) of a mutant that exhibits a bigger number of extended structures reinforces the presence of densities inside the inner tube. Arrows indicate the low-density regions that separate the cargo and inner tube. Docking of the homology model for the gp19 structure (H) supports the assignment of sheath, tube and cargo densities for the 3D models (G-J). Scale bar 10 nm.

To test whether JF50_12605 and JF50_12615 are present within the MAC complex and represent the cargo within the tube lumen, we performed protein identification by mass spectrometry of purified MACs. In two independent experiments, we detected JF50_12615 but not JF50_12605 in wildtype MAC samples (FIG. 9A). In comparison, MACs from the ΔJF50_12605 mutant exhibited fewer spectral counts for the JF50_12615 protein, in accordance with the empty phenotype observed by ECT imaging of the ΔJF50_12605 strain (FIG. 7G, S3E, or FIG. 13E). We observed a higher spectral count of JF50_12615 when the same analysis was performed on MACs from a ΔJF50_12585 (tail pin) mutant, likely because of the higher fraction of extended structures and the thus lower amount of gp19/tube and cargo that were expelled by contraction. JF50_12605 was not detected within the MAC complex (FIG. 9A). To test for protein-protein interactions between JF50_12605 and other components of the MAC complex, we utilized a bacterial two-hybrid system based on the interaction-mediated reconstruction of a cyclic AMP (CAMP) signaling cascade (Karimova et al., 2000). When JF50_12605, JF50_12615 and JF50_12680 (gp19/tube protein) were screened for interactions, we found a significant interaction between JF50_12605 and JF50_12615 as well as JF50_12605 with itself (FIG. 9B-E). However, neither JF50_12605 or JF50_12615 interacted with JF50_12680 (gp19/tube protein). Together, these data indicate that JF50_12615 is present within the MAC structure and represents the densities seen in the tube lumen while JF50_12605 might act as a mediator to localize JF50_12615 inside the gp19 tube.

FIG. 9A-D. JF50_12615 is present within MAC complexes and JF50_12605 is required for JF50_12615's association with the MAC complex:

(A) Mass spectrometry of MACs complexes. JF50_12615 was detected within MAC complexes while JF50_12605 was not. JF50_12615 was in lower abundance in MACs purified from the AJF50_12605 mutant. Higher hits to JF50_12615 and JF50_12680 (gp19/tube) were detected in ΔJF50_12585 (tailpin) mutants.

(B-D) Quantification of Bacterial Two Hybrid analyses showing interactions between JF50_12605, JF50_12615 and JF50_12680 proteins. JF50_12605 showed a strong interaction with itself and with JF50_12615.

The JF50 12615 protein is an effector sufficient for inducing metamorphosis when electroporated into Hydroides larvae:

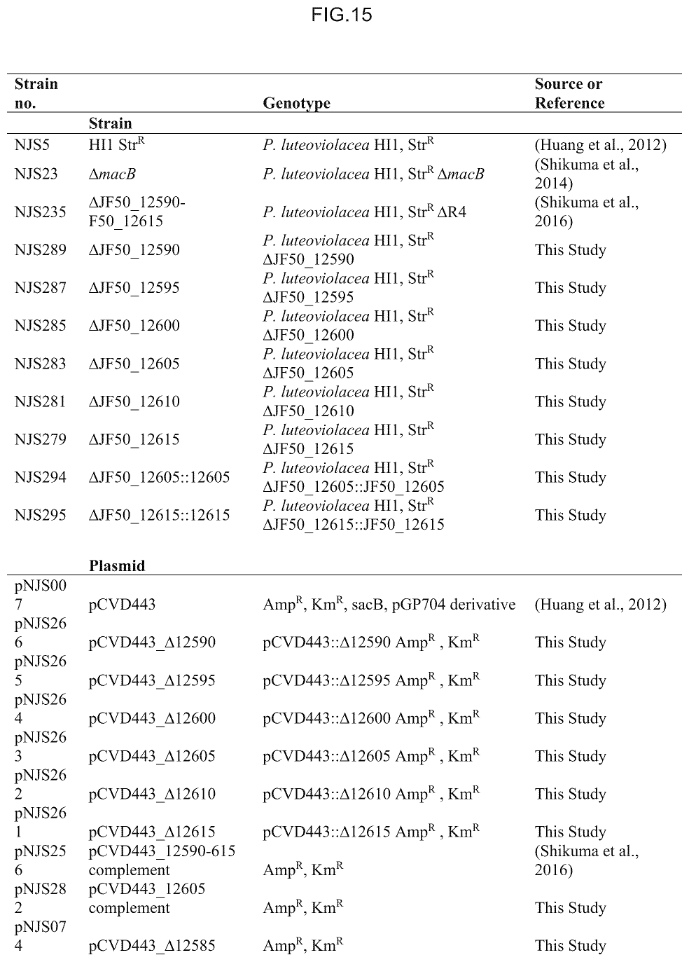

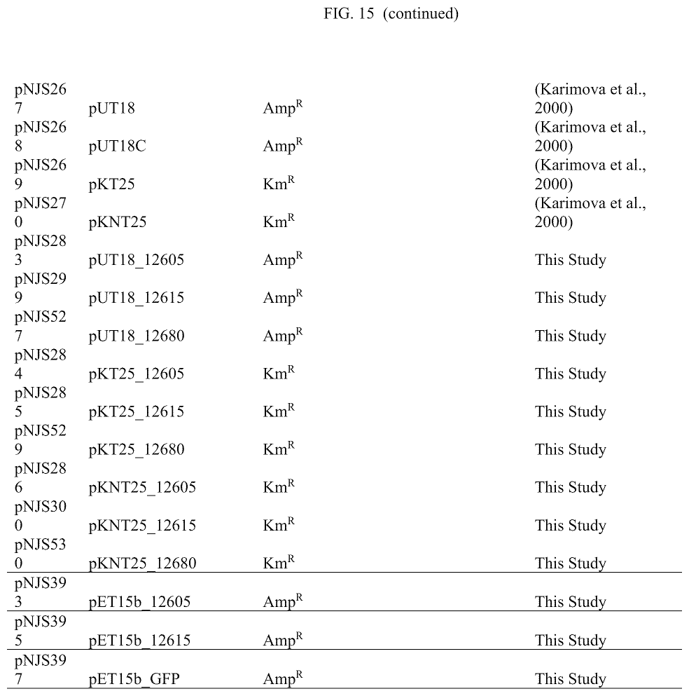

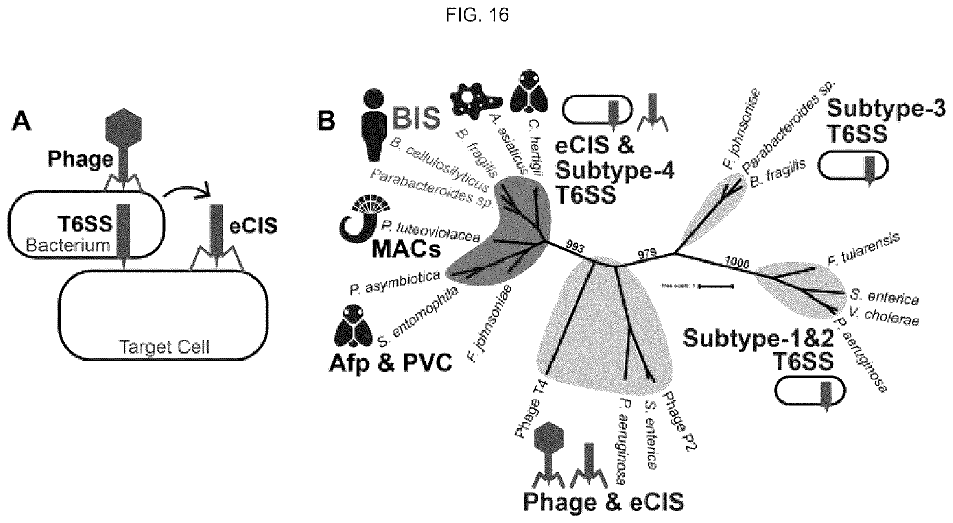

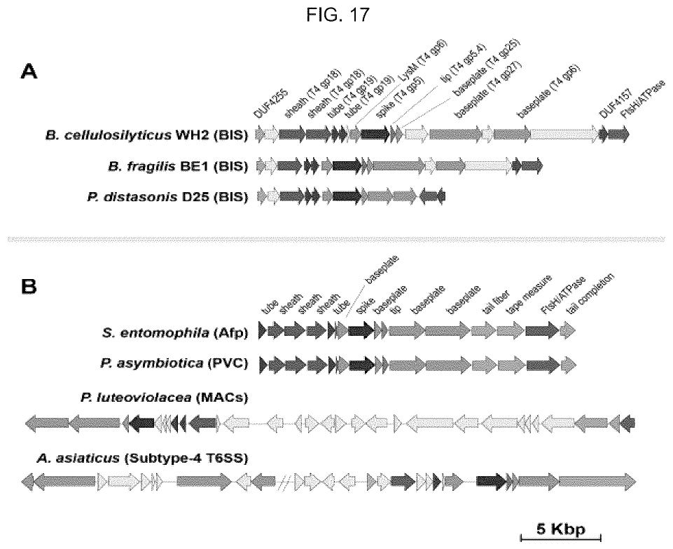

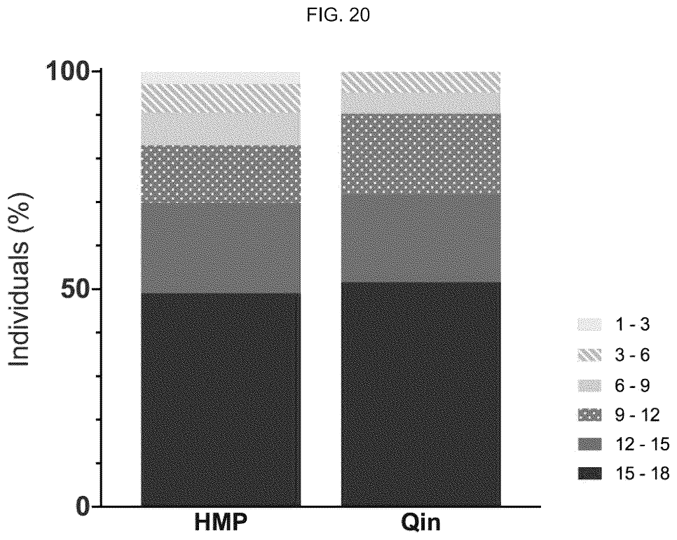

Because our results suggest that JF50_12615 is loaded within the MAC tube lumen, we next tested whether JF50_12615 is sufficient for stimulating Hydroides metamorphosis. To this end, we cloned and purified the JF50_12615 gene with an N-terminal 6xHis tag by nickel chromatography (FIG. 10A) and verified its identity by western blot with a JF50_12615-specific antibody (FIG. 10B). As controls, we cloned and purified JF50_12605 and GFP under the same conditions. JF50_12615 protein provided exogenously to competent larvae of Hydroides at concentrations up to 250 ng/μl did not stimulate metamorphosis (Fig. S4, or FIG. 14). However, we reasoned that the JF50_12615 protein might require intracellular delivery into host cells to initiate metamorphosis of Hydroides. To this end, we utilized a custom electroporator used successfully for other marine invertebrates (Zeller, 2018; Zeller et al., 2006) to deliver the JF50_12615 protein into competent Hydroides larvae at 250 ng/μl concentration. Under these conditions, a significant percentage of Hydroides larvae were induced to undergo metamorphosis (FIG. 10C). In contrast, neither JF50_12605 nor GFP stimulated metamorphosis when electroporated under the same conditions. Our results suggest that the cargo, JF50_12615, is sufficient to stimulate Hydroides metamorphosis when delivered by electroporation.

FIG. 10A-C: JF50_12615 is Sufficient for Stimulating Metamorphosis when Delivered by Electroporation:

(A) SDS page gel of purified JF50_12615, JF50_12605 and GFP.

(B) Western blot of purified JF50_12615 protein probed with a C-terminal anti-JF50_12615 peptide antibody.

(C) Metamorphosis (%) of Hydroides larvae 24 hours after electroporation with purified JF50_12615, JF50_12605 or GFP protein. Graph shows an average of biological replicates, where each point represents one biological replicate. ****p-value ≤0.0001 by t-test, ns=not significant.

FIG. 11A-B, or Figure S1. Metamorphic Response of Hydroides Larvae to Cell-Free MAC Extracts from Wild Type P. Luteoviolacea and Individual Gene Mutants:

(A) Metamorphosis (%) of Hydroides larvae 24 hours after exposure to extracted MACs from P. luteoviolacea wild type and mutants. MAC extracts were diluted 1:100 before being mixed with larvae.

(B) Dose response curve of MAC extracts from WT (red), AmacB (blue), ΔJF50_12605 (green), and ΔJF50_12615 (purple) mutants.

FIG. 12A-D, or Figure S2. Both WT and ΔJF50_12590-12615 have structurally similar arrays: MAC arrays were present in both WT (A-B) and ΔJF50_12590-12615 (C-D) MAC extracts. Each were able to form arrays that comprised individual CIS structures in both the extended and contracted conformation. Scale bar 100 nm.

FIG. 13A-F. Figure S3. Filled and empty phenotype in all studied gene mutant strains: (A-F) Slices through illustrative tomograms displaying the observed phenotype for each mutant. Scale bars are 100 nm.

FIG. 14, Figure S4. Protein Purification and JF50_12615 is unable to induce metamorphosis when added exogenously: Metamorphosis (%) of Hydroides larvae after being soaked in 250 ng/μl of purified GFP, JF50_12605, and JF50_12615 protein for 24 hours. WT MACs diluted 1:100 were used as a positive control for larval competence.

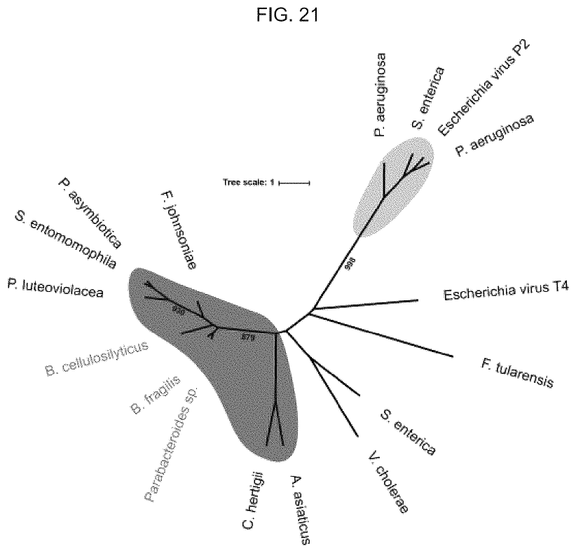

FIG. 15: illustrates strains and plasmids used in Example 2.

### Materials and Methods

Metamorphosis assays. Bioassays were conducted with specimens of Hydroides elegans obtained from Quivira Basin, San Diego, California. Embryos were obtained and maintained as previously described (Nedved and Hadfield, 2008; Shikuma et al., 2016). Competent larvae were exposed to biofilms of P. luteoviolacea wild type, as a positive control, to P. luteoviolacea mutants, and to P. luteoviolacea strains unable to produce MAC structures (ΔmacB), as well as to artificial seawater (−). The percent of larvae that underwent metamorphosis was scored 24 hours after the induction of metamorphosis. Metamorphosis was scored visually by observing the number of individuals that formed branchial radioles, and a primary and secondary tube. Four biological replicates of approximately 30 larvae each were performed for each treatment on three separate occasions with larvae spawned from different adults.
Bacterial Strains, Plasmid Construction and Culture conditions. All bacterial strains, plasmids and primer sequences used are listed in the supplemental tables S1 and S2. All deletion and fusion strains were created according to previously published protocols (Shikuma et al., 2016, 2014). Plasmid insert sequences were verified by DNA sequencing. Deletion and insert strains were confirmed by PCR. All E. coli strains were grown in Luria-Bertani (LB) media at 37° C. shaking at 200 revolutions per minute (RPM). All P. luteoviolacea cultures were grown in seawater tryptone (SWT) media (35.9 g/L Instant Ocean, 2.5 g/L tryptone, 1.5 g/L yeast extract, 1.5 ml/L glycerol) at 25° C. shaking at 200 RPM. Media that contained antibiotics were at a concentration of 100 mg/ml unless otherwise stated.
Gentle MAC extraction. P. luteoviolacea was grown in 50 ml SWT media in 250 ml flasks at 30° C. for 6 hours or overnight (12-14 h). Cells were centrifuged for 30 minutes at 4000 g and 4° C. and resuspended in 5 ml cold extraction buffer (20 mM Tris, pH 7.5, 1M NaCl). Cultures were centrifuged for 30 min at 4000 g and 4° C. and the supernatant was isolated and centrifuged for 30 minutes at 7000 g and 4° C. The pellet was resuspended in 20-100 μl cold extraction buffer and stored at 4° C. for further use.
Plunge freezing of MACs. Plunge freezing was performed as implemented in Weiss et al. (G. L. Weiss et al., 2017). In essence, gentle MAC extractions were seeded with 10 nm BSA-coated colloidal gold particles (1:4 v/v, Sigma) and 4 μl of the mixture was applied to a glow-discharged holey-carbon copper EM grid (R2/1, Quantifoil). The grid was backside blotted in a Vitrobot (FEI Company) by using a Teflon sheet on the front pad and plunge-frozen in a liquid ethane-propane mixture (37%/63%) cooled by a liquid nitrogen bath. Frozen grids were stored in liquid nitrogen.
Electron cryotomography. The gentle MAC extractions were imaged by electron cryotomography (ECT) (S. Weiss et al., 2017). Images were recorded on a Titan Krios TEM (FEI) equipped with a Quantum LS imaging filter operated at a 20 eV slit width and K2 Summit (Gatan). Pixel sizes ranged from 2.14 Å for the first batch of data in the Super Resolution (SR) mode to 2.72 Å for the remaining sessions. Tilt series were collected using a bidirectional tilt-scheme from −30° to +60° and −32° to −60° in 2° increments. Total dose was around 90 e-/Å 2 and defocus was kept from −5 to −6 μm. Some tilt series were recorded in focus using a Volta phase plate (Danev et al., 2014). Tilt series were acquired using SerialEM (Mastronarde, 2005) and reconstructed and segmented using the IMOD program suite (Kremer et al., 1996).
Sub-tomogram averaging. Tomograms used for structure identification and picking were binned by a factor of 4. Tomograms for the final reconstruction were CTF-corrected in IMOD and binned by a factor of 2 for the SR data. The discrete extended MAC structures were identified visually in individual tomograms and their longitudinal axes were modeled with open contours in 3dmod (Mastronarde, 2008). Models were generated separately for empty and filled structures from the same tomogram. Individual model points were added at defined intervals along the contours

using the addModPts program from the PEET package [25]. 4 times binned tomograms were used for a first alignment of the particles, with the aligned binned coordinates being used to initialize the final average. Tomograms for the final reconstruction were CTF-corrected in IMOD and binned by a factor of 2 for the SR data. Non-SR tomograms were CTF-corrected, exposure filtered and used unbinned for the average. For the first round of averaging studies, SR data was used to compare the wildtype filled tube phenotype (25104 particles) with the JF50_12615 deletion mutant empty phenotype (29828 particles) with a box size of 80 pixels (x) by 60 pixels (y) by 80 pixels (z) and a final pixel size of 4.28 Å. More detailed studies onto the filled tube phenotype used non-SR tomograms of the JF50_12585 deletion mutant with a total of 26215 individual model points, box size of 100 pixels (x) by 50 pixels (y) by 100 pixels (z) and a final pixel size of 2.72 Å. Subtomogram averaging was done as described in Weiss et al. (2017). UCSF Chimera (Pettersen et al., 2004) was used for visualization of the 3D models.

Bacterial Two-Hybrid Analysis. Bacterial Two-Hybrid Analysis was performed following the protocols detailed previously (Karimova et al., 2000). Briefly, proteins of interest were cloned into one of four Bacterial Two Hybrid (BTH) plasmids pUT18, pUT18C, pKT25, and pKNT25. These produced individual N- or C-terminal fusions between the proteins of interest and the T18 and T25 subunits on of the adenylate cyclase (CyaA) protein. All plasmid sequences were confirmed by PCR. Plasmid combinations containing the genes of interest were then electroporated into BTH101 electrocompetent cells that lacked a native CyaA gene. The BTH101 cells were grown on LB agar containing ampicillin (100 mg/ml), kanamycin (100 mg/ml) and 1% glucose. Glucose was used to suppress the expression of proteins before performing the assay. Protein-protein interactions were quantified by performing a β-galactosidase assay with cells being grown overnight at 37° C. and shaking at 200 RPM. Protein expression was induced with 1.0 mM IPTG. The cultures were incubated at 25° C. shaking at 200 RPM for 6 hours before being mixed with a one-step “β-gal” mix (Schaefer et al., 2016). A plate reader was then used to measure the absorbance at 420 nm and 600 nm. The optical densities were used to calculate Miller Units as previously described (Miller, 1972).
Protein Purification and Electroporation. To purify JF50_12615, JF50_12605 and GFP proteins, genes of interest were cloned into the pET15b plasmid and grown in E.coli BL21 pLysE. Bacteria were struck out on LB agar plates with ampicillin (100 μg/ul) and grown at 37ºC for 24 hours. A single colony was inoculated into 5 ml LB with ampicillin (100 μg/ul) and grown at 37° C. shaking at 200 RPM for 14-16 hours. The overnight culture was diluted 1:500 into 500 ml LB with ampicillin (100 μg/ul), grown at 37° C. shaking at 200 RPM until the culture reached an OD600 of 0.95. Protein expression was induced with 0.1 mM IPTG and grown for 25° C. for 16 hours. The culture was centrifuged at 4000 g for 20 minutes and the supernatant was removed. The pellet was then resuspended in lysis buffer (20 mM imidazole, 25 mM tris-HCl, 500 mM NaCl, pH 8) with a protease inhibitor cocktail (100 uM leupeptin, 1 uM pepstatin and 5 uM bestatin). The culture was French pressed twice (1000 psi) and sonicated 3 times for 10-30 seconds each time. The lysed culture was then spun down at 12,000 g for 20 minutes and the supernatant was discarded. Inclusion bodies were purified from the pellet by first washing the pellet twice with 20 mM tris pH 8, 2 M urea, 2% triton X-100, 500 mM NaCl. The remaining pellet was resuspended using 5 ml 6M guanidinium HCl, 5 mM imidazole, 20 mM tris pH 8, 500 mM NaCl. The 6XHIS tagged proteins were then bound to Ni-agarose beads which had been pre-equilibrated to the resuspension buffer. The proteins were refolded by adding 1 ml/min of 5 mM imidazole, 20 mM tris pH 8, 500 mM NaCl up to a total of 50 ml. After refolding, the beads were loaded onto a vacuum column and washed twice with 10 ml of refolding buffer. The protein was then eluted using 250 mM imidazole, 20 mM tris pH 8, 500 mM NaCl. Fractions containing the protein were buffer exchanged into a storage buffer (25 mM tris, 250 mM NaCl, pH 7.6) and stored at −80° C. A Bradford protein assay (BioRad) was done in order to quantify the amount of protein present. An antibody produced against the JF50_12615-specific peptide sequence CERSKGEFTEGKPKP (SEQ ID NO:48) (Genscript) was used to confirm expression and purification.

The method for electroporation of Hydroides larvae was adapted from those established for ascidian embryos (Zeller, 2018; Zeller et al., 2006). Specifically, 50 μl of 0.77 M mannitol, 20 μl of concentrated larvae (approximately 30 larvae), and 10 μl of purified protein (1.25-12.5 μg, 15.6-156 ng/μl final concentration based on protein recovery from inclusion bodies) were mixed and added to a 2 mm electroporation cuvette. The mixture was then electroporated with 30 V (15 V/cm) at 10 ohms and 3000 uF using a custom electroporation apparatus as previously described (Zeller et al., 2006). After electroporation, the mixture was immediately removed from the cuvette and mixed with 1 ml filtered artificial sea water and transferred into a 24-well plate. The larvae were then observed for metamorphosis 24 to 72 hours later (dependent on WT MACs positive control). Purification of proteins was performed on three separate occasions and each purification was electroporated twice, for a total of six independent biological replicates, each yielding similar outcomes.

Mass spectrometry. P. luteoviolacea was grown in 50 ml Marine Broth (MB) media in 250 ml flasks at 30° C. for 6 hours or overnight (12-14 hours). Cells were centrifuged for 30 minutes at 7000 g and 4° C. and resuspended in 5 ml cold extraction buffer (20 mM Tris, pH 7.5, 1 M NaCl). The resuspensions were centrifuged for 30 minutes at 4000 g and 4° C. and the supernatant was isolated and centrifuged for 30 minutes at 7000 g and 4° C. The pellet was resuspended in 20-100 μl cold extraction buffer and stored at 4° C. for further use. All mass spectrometry was done by the Functional Genomics Center Zurich (FGCZ). To prep the MAC extracts for mass spectrometry, the extracts were precipitated by mixing 30 μl of sample with 70 μl water and 100 μl 20% TCA. The samples were then washed twice with cold acetone. The dry pellets were dissolved in 45 μl buffer (10 mM Tris/2 mM CaCl₂, pH 8.2) and 5 μl trypsin (100 ng/μl in 10 mM HCl). They were then microwaved for 30 minutes at 60° C. The samples were dried, then dissolved in 20 μl 0.1% formic acid and transferred to autosampler vials for LC/MS/MS. 1 μl was injected.

Primers used in Example 2 are listed below:

| Primer | Sequence |
|---|---|
| 1556_dA | TGATGGGTTAAAAAGGATCGATCCTCTAGATTGGAGCAATAA |
| | ACGGGTTC (SEQ ID NO: 49) |
| 1556_dB | GTTCATAATTAAACTGCGATCGCAGCCATAAGGCCTCCTTGAT |
| | A (SEQ ID NO: 50) |
| 1556_dC | TATCAAGGAGGCCTTATGGCTGCGATCGCAGTTTAATTATGAA |
| | C (SEQ ID NO: 51) |
| 1556_dD | TTTTGAGACACAACGTGAATTCAAAGGGAGAGCTCCGCT |
| | TTGGGTACTGGCTTTA (SEQ ID NO: 52) |
| 1556_intF | CCGAGCAAACGTTATCACAA (SEQ ID NO: 53) |
| 1556_intR | TCAGCGCTCTCATTATGTGC (SEQ ID NO: 54) |
| 1555_dA | TGATGGGTTAAAAAGGATCGATCCTCTAGACCGAGCAAACGT |
| | TATCACAA (SEQ ID NO: 55) |
| 1555_dB | CCTTGCATGAGGTTAAGAAAGTTTGACGTACCCTTCAGCCATA |
| | TT (SEQ ID NO: 56) |
| 1555_dC | AATATGGCTGAAGGGTACGTCAAACTTTCTTAACCTCATGCA |
| | AGG (SEQ ID NO: 57) |
| 1555_dD | TTTTGAGACACAACGTGAATTCAAAGGGAGAGCTCGATGCGG |
| | TAACGGTTGTTCT (SEQ ID NO: 58) |
| 1555_intF | AGCGATTGATGCTGAACAAA (SEQ ID NO: 59) |
| 1555_intR | ACCATCGCATAACCCGTAAC (SEQ ID NO: 60) |
| 1554_dA | TGATGGGTTAAAAAGGATCGATCCTCTAGATACGCCGTCCAG |
| | TTAGGACT (SEQ ID NO: 61) |
| 1554_dB | GTTTGTTAACGTCACGGCAGCTGCATTGCCATTTAAACTCC |
| | (SEQ ID NO: 62) |
| 1554_dC | GGAGTTTAAATGGCAATGCAGCTGCCGTGACGTTAACAAAC |
| | (SEQ ID NO: 63) |
| 1554_dD | TTTTGAGACACAACGTGAATTCAAAGGGAGAGCTCATTGATT |
| | GGAAGCGCGATAG (SEQ ID NO: 64) |
| 1554_intF | TTTATGAGGCACCAACGACA (SEQ ID NO: 65) |
| 1554_intR | GCCTGTGCCGTTTTATCTGT (SEQ ID NO: 66) |
| 1553_dA | TGATGGGTTAAAAAGGATCGATCCTCTAGAGGCGATCAGTGG |
| | AGTGAAGT (SEQ ID NO: 67) |
| 1553_dB | AATACTTCTTGCTCAGCCCCGCGTGCTTCTTCTGTCATGT |
| | (SEQ ID NO: 68) |
| 1553_dC | ACATGACAGAAGAAGCACGCGGGGCTGAGCAAGAAGTATT |
| | (SEQ ID NO: 69) |
| 1553_dD | TTTTGAGACACAACGTGAATTCAAAGGGAGAGCTCTCAGAAC |
| | CAGCAGTCTCACG (SEQ ID NO: 70) |
| 1553_intF | CGGGCCTAGAAATCACTCAA (SEQ ID NO: 71) |
| 1553 intR | TCGACGTCAAATCAGTCGAG (SEQ ID NO: 72) |
| 1552_dA | TGATGGGTTAAAAAGGATCGATCCTCTAGAGAGAGCAAGAAG |
| | TGGCGAGT (SEQ ID NO: 73) |
| 1552_dB | TAGCCTTTTAGTGCCGCTTTTGAGGCGTCCATATCTGACA |
| | (SEQ ID NO: 74) |
| 1552_dC | TGTCAGATATGGACGCCTCAAAAGCGGCACTAAAAGGCTA |
| | (SEQ ID NO: 75) |
| 1552_dD | TTTTGAGACACAACGTGAATTCAAAGGGAGAGCTCTGCTGACC |
| | AAGCAGATTGAC (SEQ ID NO: 76) |
| 1552_intF | GGGCAATTGTTGTGGATTTT (SEQ ID NO: 77) |
| 1552_intR | TGATCCCAAACCACTTGTGA |
| | TGATGGGTTAAAAAGGATCGATCCTCTAGAGACTGCTGG |
| | (SEQ ID NO: 78) |
| 1551_dA | TTCTGATTCGAT (SEQ ID NO: 79) |
| 1551_dB | AACAGATCATTACATTAAAATGAGCCTCTGTTCTTGTTGTTGC |
| | ATTTCA (SEQ ID NO: 80) |
| 1551_dC | TGAAATGCAACAACAAGAACAGAGGCTCATTTTAATGTAATG |
| | ATCTGTT (SEQ ID NO: 81) |
| 1551 dD | TTTTGAGACACAACGTGAATTCAAAGGGAGAGCTCCTTCTCCA |
| | TTTTCGCCTTTG (SEQ ID NO: 82) |
| 1551_intF | CGTTTTCAGTGACCATCACG (SEQ ID NO: 83) |
| 1551_intR | CGGTGGGCAAAAAGGTATAA (SEQ ID NO: 84) |
| pUT18_605_F1 | CAGCTATGACCATGATTACGCCAAGCTTGCATGCCATGACAG |
| | AAGAAGCACGCGAAAAAA (SEQ ID NO: 85) |
| pUT18_605_R1 | CTGGCGGCTGAATTCGAGCTCGGTACCCGGGGATCATTCACA |
| | AGTGCTAATTGATAAAAT (SEQ ID NO: 86) |
| pUT18_615_F1 | CAGCTATGACCATGATTACGCCAAGCTTGCATGCCATGCAAC |
| | AACAAGAACAGGAGCAAG (SEQ ID NO: 87) |
| pUT18_615_R1 | CTGGCGGCTGAATTCGAGCTCGGTACCCGGGGATCCATTAAA |
| | ATGAGCCTTTCTTTTTCA (SEQ ID NO: 88) |
| pUT18_680_F | CATGATTACGCCAAGCTTGCATGCCATGGCTACTACTAAAGC |
| | AGATATCG (SEQ ID NO: 89) |
| pUT18_680_R | AATTCGAGCTCGGTACCCGGGGATCATGGAACTCAATCTTGA |
| | TGTCATCT (SEQ ID NO: 90) |
| pKT_605_F1 | CCGATTACCTGGCGCGCACGCGGCGGGCTGCAGGGATGACA |
| | GAAGAAGCACGCGAAAAAA (SEQ ID NO: 91) |
| pKT_605_R1 | AACGACGGCCGAATTCTTAGTTACTTAGGTACCCGCTAATTC |
| | ACAAGTGCTAATTGATAA (SEQ ID NO: 92) |
| pKT_615_F1 | CCGATTACCTGGCGCGCACGCGGCGGGCTGCAGGGATGCAAC |
| | AACAAGAACAGGAGCAAG (SEQ ID NO: 93) |
| pKT_615_R1 | AACGACGGCCGAATTCTTAGTTACTTAGGTACCCGTTACATT |
| | AAAATGAGCCTTTCTTTT (SEQ ID NO: 94) |
| pKT_680_F1 | CCTGGCGCGCACGCGGCGGGCTGCAATGGCTACTACTAAAGC |
| | AGATATCG (SEQ ID NO: 95) |
| pKT_680_R1 | GCCGAATTCTTAGTTACTTAGGTACTTAATGGAACTCAATCTT |
| | GATGTCA (SEQ ID NO: 97) |
| pKNT_605_F1 | CAGCTATGACCATGATTACGCCAAGCTTGCATGCCATGACAGA |
| | AGAAGCACGCGAAAAAA (SEQ ID NO: 98) |
| pKNT_605_R1 | TGATGCGATTGCTGCATGGTCATTGAATTCGAGCTATTCACAA |
| | GTGCTAATTGATAAAAT (SEQ ID NO: 99) |
| pKNT_615_F1 | CAGCTATGACCATGATTACGCCAAGCTTGCATGCCATGCAACA |
| | ACAAGAACAGGAGCAAG (SEQ ID NO: 100) |
| pKNT_615_R1 | TGATGCGATTGCTGCATGGTCATTGAATTCGAGCTCATTAAAA |
| | TGAGCCTTTCTTTTTCA (SEQ ID NO: 101) |
| pKNT_615_F2 | CATGATTACGCCAAGCTTGCATGCCATGCAACAACAAGAACA |
| | GGAGCAAG (SEQ ID NO: 102) |
| pKNT25-680_F | CATGATTACGCCAAGCTTGCATGCCATGGCTACTACTAAAGCA |
| | GATATCG (SEQ ID NO: 103) |
| pKNT25-680_R | GCTGCATGGTCATTGAATTCGAGCTATGGAACTCAATCTTGAT |
| | GTCATCT (SEQ ID NO: 104) |
| pET15b_605_F1 | TGCCGCGCGGCAGCCATATGATGACAGAAGAAGCACGCG |
| | (SEQ ID NO: 105) |
| pET15b_605_R1 | GCTTTGTTAGCAGCCGGATCCCTAATTCACAAGTGCTAATT |
| | (SEQ ID NO: 106) |

### Discussion

To date, identified bacterial cues for animal metamorphosis can all be classified as small molecules (Sneed et al., 2014; Swanson et al., 2007; Tebben et al., 2011), rather than effectors injected by a bacterial secretion system. Our discovery that P. luteoviolacea's syringe-like MACs induce the metamorphosis of Hydroides larvae by injecting a proteinaceous effector into animal cells forces us to expand the scope of possible biomolecules and mechanisms by which bacteria stimulate animal development. Additionally, our results confirm a previously hypothesized effector loading and delivery mechanism within the tube lumen of a CIS (Shneider et al., 2013).

Based on the present work, we name JF50_12615 as Mif1 for Metamorphosis-Inducing Factor 1. While MACs benefit tubeworms because they induce metamorphosis, it is currently unclear whether there is a benefit for the producing bacterium. Mif1 does not possess any predicted domains, yielding no clues to the mechanism of its function. While the stimulation of metamorphosis has been hypothesized to be induced by the depolarization of a larval cell membrane (Carpizo-Ituarte and Hadfield, 1998; Yool et al., 1986), the identification of Mif1 argues against the possibility of a solely physical disruption to a larval cell membrane by the MACs' syringe-like action stimulating metamorphosis. While we do not know how Mif1 stimulates Hydroides metamorphosis, Mif1 could possess the ability to form pores in target-cell membranes, leading to membrane depolarization and triggering metamorphosis. Alternatively, Mif1 might induce metamorphosis by an inherent enzymatic activity as is the case for other CIS effectors targeting eukaryotic cells (Jiang et al., 2016; Ma and Mekalanos, 2010; Vlisidou et al., 2019).

As micron scale-sized, syringe-like structures, MACs have potential for being developed as peptide delivery systems for eukaryotic cells. Extracellular CIS (eCIS) like MACs are of particular interest because they are released from the producing bacterial cell and autonomously bind to the target cell's surface. eCIS that target bacterial pathogens are already under development as narrow host-range antimicrobial agents (Scholl, 2017). The identification of effectors carried by MACs provides the basis for modification of MACs' cargo for biotechnology purposes. Recently, we reported that MACs carry a second effector with nuclease activity called Pne1 that targets eukaryotic cells, and, intriguingly, Pne1 does affect Hydroides metamorphosis (Rocchi et al., 2019). Future manipulation of the tail fibers and/or tail pins MACs use for targeting cells and its effector contents could lead to the development of cell-specific injection machines loaded with a custom payload.

While bacteria are known to stimulate metamorphosis in every major group of animals alive today (Hadfield, 2011), the identity of bacterial factors and their modes of action remain poorly understood. The identification of Mif1 and its delivery mechanism from within the tube lumen of a CIS will (1) facilitate our study of how bacterial factors trigger animal signaling systems leading to metamorphosis and (2) have potential practical applications for preventing biofouling, improving aquaculture husbandry, restoring degraded ecosystems like coral reefs and as a biotechnology platform. The scope of possibilities by which bacteria interact with animals via MAC-like structures is immense, because genes encoding evolutionarily related CIS are found in microbes from diverse environments including the ocean, terrestrial environments and even the human gut (Böck et al., 2017; Sarris et al., 2014). Ongoing work studying the mechanism by which Mif1 stimulates metamorphosis will provide insight into a form of bacteria-animal interactions with implications for both bacteria and animal biology.

### Example 3: Distinct Contractile Injection System Found in a Majority of Adult Human Microbiomes

This example provides exemplary methods for making compositions and bacterial cells as provided herein.

An imbalance of normal bacterial groups such as Bacteroidales within the human gut is correlated with diseases like obesity. A current grand challenge in the microbiome field is to identify factors produced by normal microbiome bacteria that cause these observed health and disease correlations. While identifying factors like a bacterial injection system could provide a missing explanation for why Bacteroidales correlates with host health, no such factor has been identified to date. The lack of knowledge about these factors is a significant barrier to improving therapies like fecal transplants that promote a healthy microbiome. Here we show that a previously ill-defined Contractile Injection System is carried in the gut microbiome of 99% of individuals from the United States and Europe. This type of Contractile Injection System, we name here Bacteroidales Injection System (BIS), is related to the contractile tails of bacteriophage (viruses of bacteria) and have been described to mediate interactions between bacteria and diverse eukaryotes like amoeba, insects and tubeworms. Our findings that BIS are ubiquitous within adult human microbiomes suggest that they shape host health by mediating interactions between Bacteroidales bacteria and the human host or its microbiome.

The human gut harbors a dynamic and densely populated microbial community¹. The gut microbiome of healthy individuals is characterized by a microbial composition where members of the -Bacteroidetes phylum (Bacteroides and Parabacteroides) constitute 20-80% of the total². Several studies have demonstrated that dysbiosis in the human gut is correlated with microbiome immaturity, and diseases like obesity and inflammatory bowel disease³⁻⁷. However, we currently do not know the identity of bacterial factors that explain these correlations between Bacteroidales abundances and healthy human phenotypes. The lack of knowledge about these factors is a significant barrier to improving therapies like fecal transplants that manipulate microbial communities to treat microbiome-related illness.

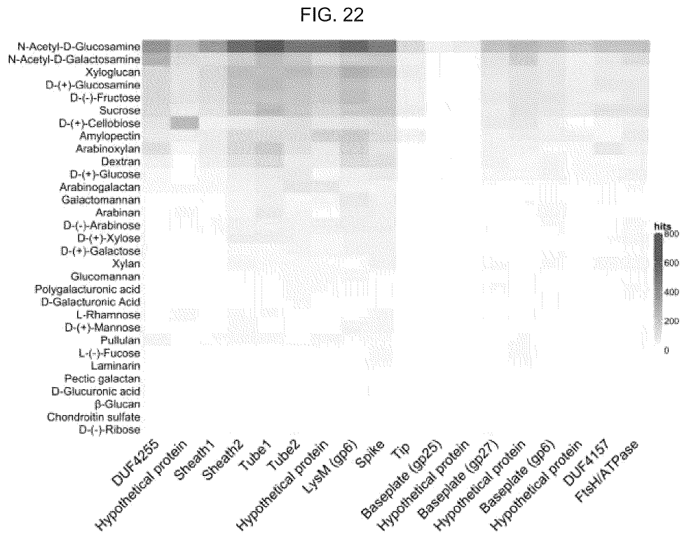

Many bacteria such as Bacteroidales produce syringe-like secretion systems called Contractile Injection Systems (CIS) that are related to the contractile tails of bacteriophage (bacterial viruses)^{8,9}. Most CIS characterized to date, termed Type 6 Secretion Systems (T6SS), are produced and act from within an intact bacterial cell. In contrast, extracellular CIS (eCIS) are released by bacterial cell lysis, paralleling the mechanism used by tailed phages to escape their host cell (FIG. 16A). To date, Bacteroidales from the human gut have only been shown to produce one type of CIS, a Subtype-3 T6SS that mediates bacteria-bacteria interactions and can govern the microbial composition of the gut microbiome¹⁰⁻¹³. However, CIS from other bacterial groups such as Gammaproteobacteria are known to help bacteria interact with diverse eukaryotic organisms such as amoeba, insects, tubeworms and humans.

Distinct from Bacteroidales Subtype-3 T6SS is a different class of CIS that may have evolved independently¹⁴. Intriguingly, all previously described examples of these distinct CIS mediate bacteria-eukaryote interactions. Three are classified as eCIS and include: (1) MACs (Metamorphosis Associated Contractile Structures) that stimulate the metamorphosis of tubeworms^{15,16,} (2) PVCs (Photorhabdus Virulence Cassettes) that mediate virulence in grass grubs¹⁷, and (3) Afp (Anti-Feeding Prophage) that cause cessation of feeding and death of grass grub larvae¹⁸⁻²¹. A fourth CIS from Amoebophilus asiaticus¹⁴ promotes intracellular survival in amoeba and defines the Subtype-4 T6SS group. However, until the present work, examples of this distinct class of CIS have only been identified in a few isolated Bacteroidetes genomes^{14,22,23}.

In this study, we show that diverse Bacteroidales species from the human gut encode a distinct CIS within their genome that is related to eCIS and T6SS that mediate tubeworm, insect and amoeba interactions (MACs, PVCs, Afp, Subtype-4 T6SS). Strikingly, we show that this distinct CIS from Bacteroidales are present within the microbiomes of over 99% of human adult individuals from Western countries (Europe and the United States) and are expressed in vivo. Our results suggest that a previously enigmatic class of contractile injection system is present and expressed within the microbiomes of nearly all adults from Western countries.

### Results

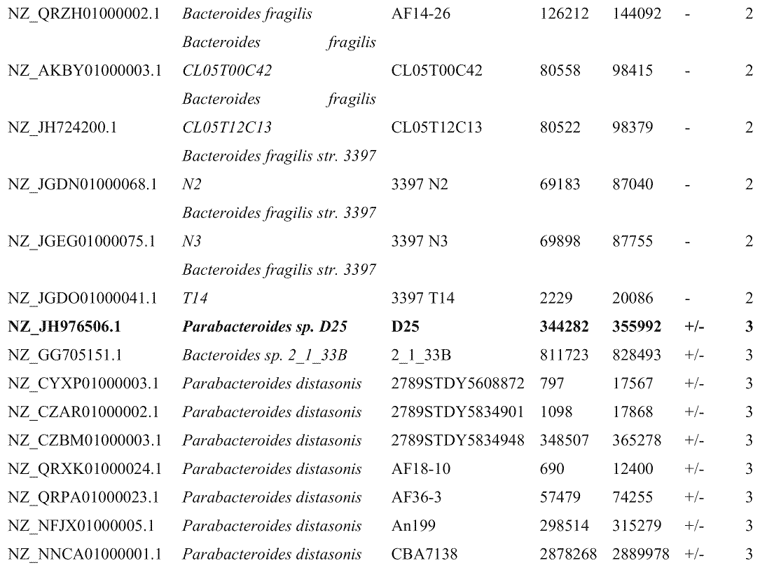

Bacteroidales bacteria from the human gut possess genes encoding a distinct Contractile Injection System. Using PSI-BLAST to compare previously identified eCIS and Subtype-4 T6SS proteins to the non-redundant (nr) protein sequence database, we identified CIS structural proteins (baseplate, sheath and tube) that matched with proteins from various human Bacteroidales isolates, including a bacterial isolate from the human gut (Bacteroides cellulosilyticus WH2, Table S1 illustrated in FIG. 23)²⁴.

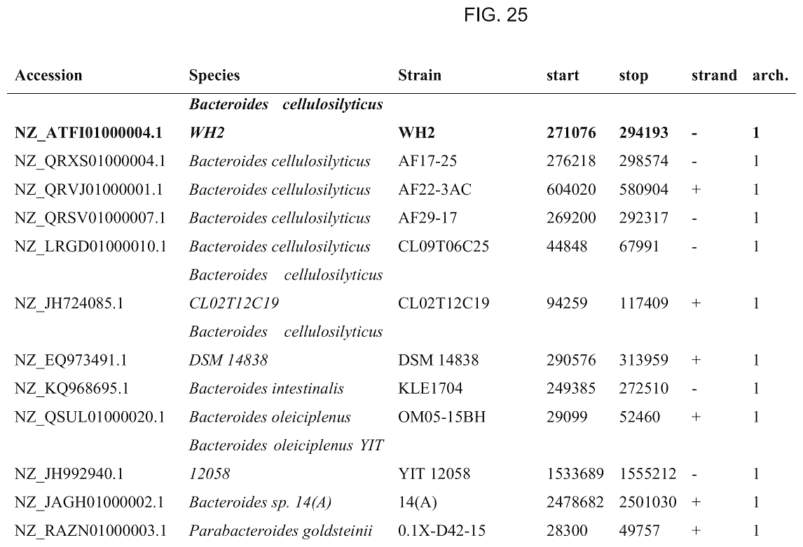

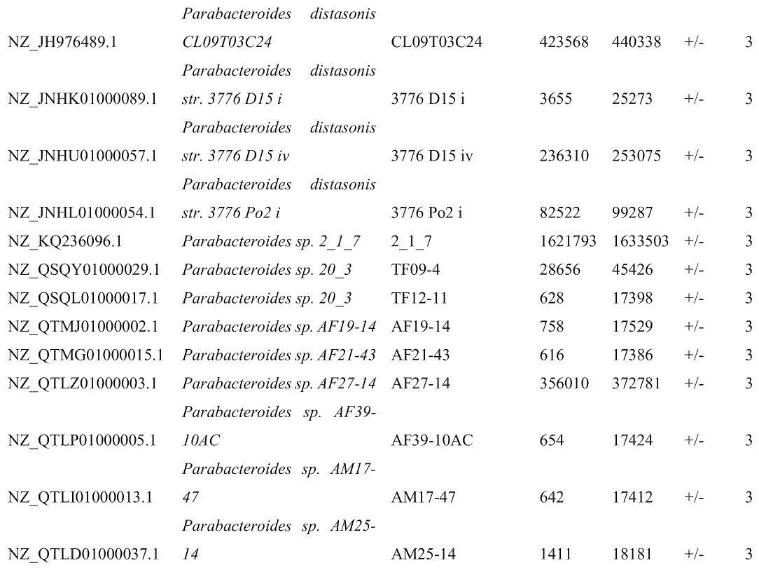

To determine the relatedness of these distinct CIS with all known CIS subtypes, we performed phylogenetic analyses of CIS proteins that are key structural components of CIS-the CIS sheath and tube. Multiple methods of phylogenic analyses (maximum likelihood, neighbor joining, maximum parsimony, UPGMA, and minimum evolution) showed that Bacteroidales sheath and tube proteins consistently formed a monophyletic group with other eCIS and Subtype-4 T6SS sheath and tube proteins (FIG. 16B, FIG. 21, Table S2 illustrated in FIG. 24). Moreover, the BIS sheath and tube were clearly distinct when compared to previously characterized Bacteroides Subtype-3 T6SS (FIG. 16B)^{10,11}. Based on these data and results below, we name this distinct CIS as Bacteroidales Injection Systems (BIS).

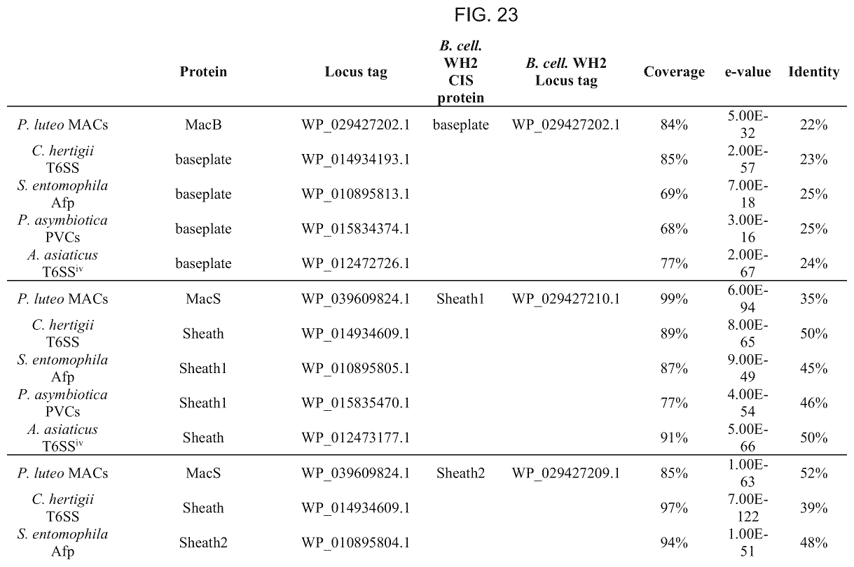

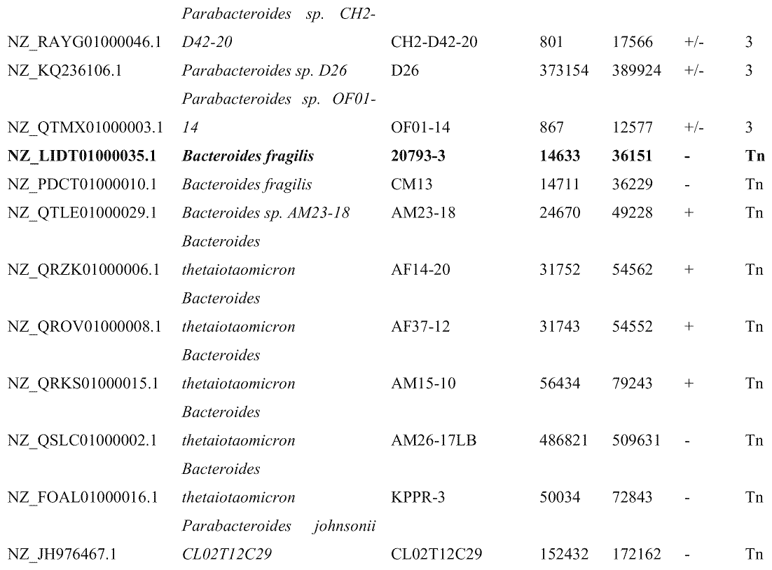

Genes encoding BIS are found in a conserved cluster of genes and form three genetic arrangements. To identify Bacteroidales species that possess a bonafide BIS gene cluster, we performed a comprehensive search of 759 sequenced Bacteroides and Parabacteroides genomes from the Refseq database. Our sequence-profile search revealed 66 genomes from Bacteroides and Parabacteroides species that harbor complete BIS gene clusters (Table S3 as illustrated in FIG. 25) in three conserved gene arrangements (FIG. 17A). The first architecture is exemplified by B. cellulosilyticus WH2, which harbors two sheath proteins, two tube proteins, and a protein with unknown function intervening between putative genes encoding the baseplate (gp25, gp27 and gp6). The second architecture is exemplified by B. fragilis BE1. This architecture has a single sheath protein and lacks the hypothetical proteins observed in architecture one between gp25 and gp27, and between Tube2 and LysM. Finally, the third architecture defined by P. distasonis D25 is the most compact, lacks four hypothetical proteins found in architectures 1 and 2. Additionally, gp27 and gp6 proteins are shorter, and the genes FtsH/ATPase and DUF4157 are inverted. Importantly, all three genetic architectures have genes with significant sequence similarity to MAC, Afp and PVC genes shown previously to produce a functional CIS, including baseplate proteins (gp25, gp27, and gp6), sheath, tube, and FtsH/ATPase (FIG. 17B).

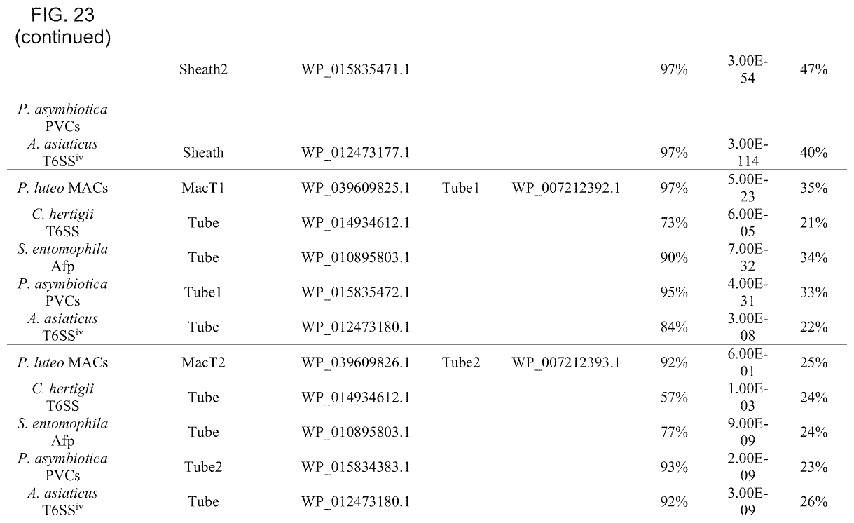

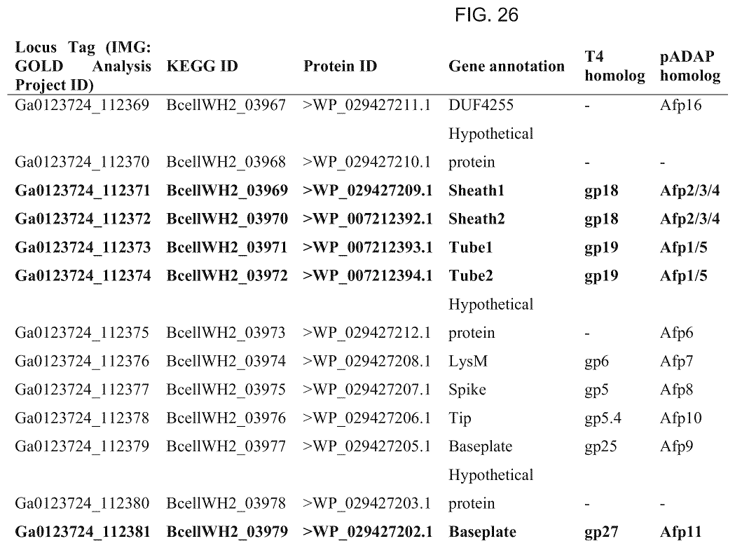

BIS genes are present in human gut, mouth, and nose microbiomes. To determine the prevalence and distribution of BIS genes in human microbiomes, we searched shotgun DNA sequencing data from 11,219 microbiomes from the Human Microbiome Project database, taken from several locations on the human body^{2,25}. We sampled these metagenomes for the presence of 18 predicted BIS proteins (Table S4 as illustrated in FIG. 26). Across all HMP metagenomes, 8,320 (74%) showed hits to at least one of the 18 BIS proteins. Hits were distributed across metagenomes from various mucosal tissues, and were more abundant in the gut and in the mouth, where Bacteroidales are found in high abundance². The dataset included stool (1,851, 99.6% of total stool metagenomes), oral (4,739, 79.2% of total oral metagenomes), nasal (630, 41.8% of total nasal metagenomes), and vaginal (232, 27.7% of total vaginal metagenomes) samples (FIG. 18A).

To determine how often any of the 18 genes co-occurred within the same metagenome sample, we constructed a co-occurrence network. Ten genes appeared together at high frequencies including Sheath1, Sheath2, FtsH/ATPase, baseplate (gp25, gp27, and gp6), LysM, Spike, and two hypothetical proteins (FIG. 18B). The gene with the highest hit abundance encodes for an ATPase homologous to the Escherichia coli FtsH, known to be involved in cleavage of the lambda prophage repressor, followed by a hypothetical protein, and Sheath1. The other genes, including Tube1, Tube2, Tip, DUF4255 domain-containing protein, DUF4157 domain-containing protein, and three hypothetical proteins, were detected together less often within the microbiome samples.

BIS genes are expressed in vivo in the gut of humanized mice, and in vitro when cultured with various polysaccharides. To determine whether BIS genes are transcribed under laboratory growth conditions or within the human gut, we searched for the 18 major BIS proteins in publicly available RNA sequencing data from in vivo metatranscriptomes of humanized mice²⁶ (gnotobiotic mice colonized with human microbiome bacteria) and in vitro B. cellulosilyticus WH2 pure cultures²⁴. We inspected 59 metatranscriptomes from a previously published in vivo study²⁶ for the presence of the 18 major BIS proteins, where gnotobiotic mice were inoculated with human gut microbiome cultures. In 48 out of 59 metatranscriptomes (81.4%), we found expression of at least 15 BIS proteins (FIG. 4). Similarly, when B. cellulosilyticus WH2 was cultured in minimum media (MM) supplemented with 31 different simple and complex sugars²⁴, all 18 genes were expressed at least once in at least two of the three replicate cultures (FIG. 21). The highest expression was seen under growth in N-Acetyl-D-Galactosamine (GalNAc) and N-Acetyl-Glucosamine (GlcNAc), amino sugars that are common components of the bacterial peptidoglycan, in high abundance in the human colon, and implicated in many metabolic diseases^{27,28}. Our analyses of metatranscriptomes show that BIS genes are transcribed by Bacteroidales bacteria under laboratory growth conditions and within humanized mouse microbiomes in vivo.

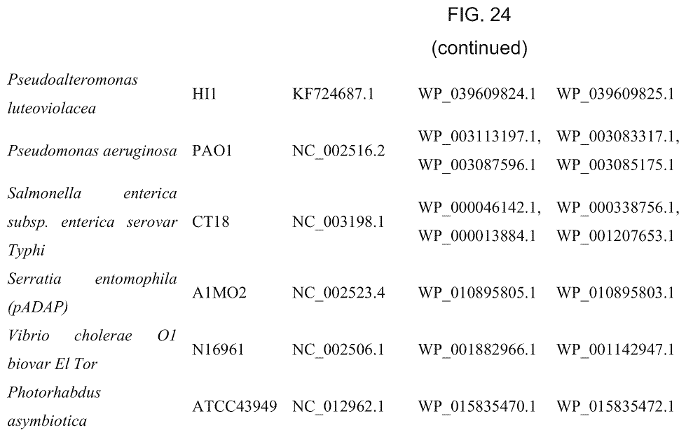

BIS genes are present in the microbiomes of nearly all adult individuals. To determine the prevalence of BIS within the microbiomes of human populations, we analyzed 2, 125 fecal metagenomes from 339 individuals; 124 Individuals from Europe²⁹ and 215 individuals from North America². We found that all individuals possessed at least 1 of the 18 BIS genes within their gut microbiome (FIG. 20). Most individuals carried at least 9 BIS proteins (83.0% in HMP and 90.3% in Qin dataset). A lower number possessed all 18 BIS proteins (8.96% in HMP and 6.45% in Qin dataset).

### Discussion

Here, we show that a previously ill-described Contractile Injection System is present in the gut microbiomes of nearly all adult individuals from Western countries. We found that BIS genes are present in human microbiomes throughout mucosal tissues (oral, nasal, vaginal, ocular) and enriched in metagenomes from gut samples. CIS have gained recent recognition as secretion systems promoting disease in several prominent human pathogens like Pseudomonas aeruginosa and Vibrio cholerae. However, our discovery of a distinct class of CIS produced by Bacteroidales from human microbiomes stems from studies of symbiotic interactions between environmental bacteria and diverse eukaryotic hosts like tubeworms, insects and amoeba^{14-16,30}. The close relatedness of BIS with other structures promoting microbe-eukaryotic interactions (MACs, Afp, PVCs and Subtype-4 T6SS) suggests that BIS mediate interactions between bacterial species within the human microbiome or Bacteroidales bacteria and their human host.

If BIS do interact with human cells, they may promote either symbiotic or pathogenic interactions. Injection systems closely related to BIS are described to mediate both beneficial and infectious microbe-host relationships. For example, MACs mediate metamorphosis of a marine tube worms^{15,31} and a Subtype-4 T6SS¹⁴ mediates membrane interaction between A. asiaticus and its amoeboid host. In contrast, Afp and PVCs inject toxic effectors into insects^{17,21}. While a Subtype-3 T6SS has been described in Bacteroides bacteria, we speculate that BIS could be the first Bacteroidales CIS to mediate microbe-animal interactions.

Structures that are closely related to BIS have been described to form T6SS that act from within a bacterial cell (e.g. Subtype-4 T6SS from A. asiaticus; Böck et al., 2017) or eCIS that are released by cell lysis (e.g. MACs and PVCs; Shikuma et al., 2014; Vlisidou et al., 2019). While we currently do not know whether BIS generate a T6SS and/or an eCIS structure, we do know that intestinal bacteria like Bacteroides are physically separated from the intestinal epithelium by a layer of mucus³²⁻³⁴. In contrast to T6SS that are bound to and act from within bacterial cells, an eCIS from Bacteroidales could cross this mucus barrier to inject effectors into human epithelial cells.

We show here that BIS are produced by Bacteroidales in vivo during colonization of humanized mice²⁶ and under laboratory conditions with various carbon sources²⁴. However, we do not yet know the conditions that promote BIS production within normal or diseased human individuals. BIS may not have been extensively described before this work because they likely evolved independently from previously described CIS like Subtype-3 T6SS^{13,14} and possess significantly divergent sequence homologies (FIG. 16B). Like other described CIS, BIS gene clusters possess genes encoding the syringe-like structural components and could encode for effectors that elicit specific cellular responses from target host cells. For example, the closely related injection system called MACs possesses two different effectors; one effector protein promotes the metamorphic development of a tubeworm³¹ and a second toxic effector kills insect and mammalian cell lines³⁵.

Many correlations between Bacteroidales abundances in the human gut and host health are currently unexplained. Future research into the conditions that promote the production BIS and its protein effectors will yield new insight into how Bacteroidales abundances correlate with host health, potentially by promoting the direct interaction between the microbiome and human host. In addition to their effect on host health, BIS provide the tantalizing potential as biotechnology platforms because they may be manipulated to inject engineered proteins of interest into other microbiome bacteria or directly into human cells.

### Materials and Methods

Phylogenetic analyses of CIS structural proteins. Whole genomes and assembled contigs of representative phage-like clusters (Table S2) were downloaded from NCBI to construct a database using BLAST+(2.6.0). The B. cellulosilyticus WH2 Sheath1 (WP 029427210.1) and Tubel (WP 118435218) protein sequences were downloaded from NCBI and a tBLASTn was performed against the genome database. The recovered nucleotide sequences were then translated using EMBOSS Transeq (EMBL-EBI, https://www.ebi.ac.uk/Tools/st/emboss_transeq/) to generate a list of protein sequences. To capture highly divergent protein homologs, we used T6SS-Hcp, VipA/B; Phage-gp18, gp19 as reference proteins. The amino acid sequences were aligned using the online version of MAFFT (v7) with the iterative refinement alignment method E-INS-i. The aligned fasta file was converted into a phylip file using Seaview³⁶. PhyML was performed through the ATCG Bioinformatics web server and utilized the Smart Model Selection (SMS) feature and the Maximum-Likelihood method^{37,38}. The best models for the Sheath1 and Tube1 phylogenies were rtREV+G+F and LG+G+I+F, respectively. Bootstrap values (1000 resamples) were calculated to ensure tree robustness. The Maximum likelihood tree topology was confirmed using other methods, including Neighbor joining, Maximum-Parsimony, Minimum-Evolution, and UPGMA. Trees were manipulated and viewed in iTOL³⁹.

BIS gene cluster synteny analyses. To identify CIS gene clusters in Bacteroidetes we used a modified protocol used to identify T6SS¹³. Briefly, the assemblies for 759 Bacteroides and Parabacteroides genomes included in the Refseq database (release 92, 26553804) were downloaded. Proteins from assembly were searched with HHMER v3.2.1 (http://hmmer.org/) for a match above the gathering threshold of Pfam HMM profile ‘phage_sheath_1’ (PF04984)⁴⁰. For each match, up to 20 proteins were extracted from either side. All proteins from the resulting set (phage sheath±20 proteins) were sorted by length and clustered at 50% amino acid identity using UClust v1.2.22q⁴¹. Clusters containing ≥4 members were analyzed further. Cluster representatives were annotated using protein-profile searches against three databases: the Pfam-A database using HMMER3⁴⁰, the NCBI Conserved Domain Database using RPS-BLAST⁴²⁻⁴⁴, and the Uniprot30 database (accessed February 2019, available from http://wwwuser.gwdg.de/˜compbiol/data/hhsuite/databases/hhsuite_dbs/) using HHblits^{45,46}. Multiple sequence alignments were automatically generated from three iterations of the HHblits search and used for profile-profile comparisons against the PDB70 database (accessed February 2019, available from http://wwwuser.gwdg.de/˜compbiol/data/hhsuite/databases/hhsuite_dbs/). Significant hits to cluster representatives were used to assign an annotation to all proteins contained within the parent cluster. Manual inspection of Bacteroides and Parabacteroides loci enabled consistent trimming of each genetic architecture; specifically, the genes intervening DUF4255 and FtsH/ATPase were retained.

### Metagenomic Mining Analyses

To find the prevalence of the BIS genes within the Human Microbiome Project, we downloaded 11219 metagenomes using NCBI's fastq-dump API. The metagenomes were parsed where: left-right tags were clipped, technical reads (adapters, primers, barcodes, etc) were dropped, low quality reads were dropped, and paired reads were treated as two distinct reads. A subject database was created from the amino-acid sequences of the 18 BIS genes. Then the fastq files were piped through seqtk⁴⁷, to convert them to fasta format, which was then piped to DIAMOND via stdin. Then DIAMOND aligned the six-frame translation of the input reads against the subject database, with all default parameters. For each metagenome the number of non-mutually exclusive hits to each CIS gene were then summed providing a hit ‘count score’. From the hit counts, a heatmap was created by taking the number of hits of each gene per metagenome and dividing by the total number of reads and multiplying the result by one million, which was then log10(x+1) transformed. To estimate the co-occurrence between pairs of genes, the previous the hit count scores from the previous calculation were taken, and for each pair combination, the hit count of the lower of the two genes was added to a running total. The co-occurrence was then visualized on a network graph, where each edge corresponds to the number of times the pair of genes co-occurred in all the metagenomes^{48,49} (R core Team 2017—https://www.R-project.org/; ggraph—https://CRAN.R-project.org/package=ggraph).

Fastq files from transcriptomes were downloaded from the Sequence Read Archive using the sra toolkit (https://www.ncbi.nlm.nih.gov/sra/docs/toolkitsoft/). Low quality reads were removed using prinseq++ (https://peerj.com/preprints/27553/). Reads were compared to the amino acid sequences of the Bacteroides cellulosilyticus WH2 BIS protein cluster using blastX with an E-value cutoff of 0.001. The best hit for each read was kept. Hits to each protein were normalized by the number of reads of each transcriptome and the length of each protein using the program Fragment Recruitment Assembly Purification (https://github.com/yinacobian/frap).

### References—Example 1

Bladergroen M R, et al. 2003. Infection-Blocking Genes of a Symbiotic Rhizobium leguminosarum Strain That Are Involved in Temperature-Dependent Protein Secretion. Mol Plant-Microbe Interact 16:53-64.
Böck D, et al. 2017. In situ architecture, function, and evolution of a contractile injection system. Science 357:713-717.
Gebhart D, et al. 2015. A Modified R-Type Bacteriocin Specifically Targeting Clostridium difficile Prevents Colonization of Mice without Affecting Gut Microbiota Diversity. MBio 6:e02368-14.
Ho B T, et al. 2014. A view to a kill: the bacterial type 6 secretion system. Cell Host Microbe 15:9-21.
Holmstrom C, Kjelleberg S. 1999. Marine Pseudoalteromonas species are associated with higher organisms and produce biologically active extracellular agents. FEMS Microbiol Ecol 30:285-293.
Huang Y, et al. 2012. Recruitment in the sea: Bacterial genes required for inducing larval settlement in a polychaete worm. Sci Rep 2.
Hurst M R H, et al. 2007. Isolation and characterization of the Serratia entomophila antifeeding prophage. FEMS Microbiol Lett 270:42-48.
Hurst M R H, et al. 2004. Cloning Serratia entomophila antifeeding genes—a putative defective prophage active against the grass grub Costelytra zealandica. J Bacteriol 186:5116-28.
Jiang N, et al. 2018. Vibrio parahaemolyticus RhsP represents a widespread group of pro-effectors for type VI secretion systems. Nat Commun 9.
Kelley L A, et al. 2015. The Phyre2 web portal for protein modeling, prediction and analysis. Nat Protoc 10:845-858.
Korczynska J E, Turkenburg J P, Taylor E J. 2012. The structural characterization of a prophage-encoded extracellular DNase from Streptococcus pyogenes. Nucleic Acids Res 40:928-38.
Koskiniemi S, et al. 2013. Rhs proteins from diverse bacteria mediate intercellular competition. Proc Natl Acad Sci 110:7032-7037.
Lien Y -W, Lai E -M. 2017. Type VI Secretion Effectors: Methodologies and Biology. Front Cell Infect Microbiol 7:1-11.
Ma L S, et al. 2014. Agrobacterium tumefaciens deploys a superfamily of type VI secretion DNase effectors as weapons for interbacterial competition in planta. Cell Host Microbe 16:94-104.
Mastronarde D N. 2005. Automated electron microscope tomography using robust prediction of specimen movements. J Struct Biol 152:36-51.
Mastronarde D N. 1997. Dual-Axis Tomography: An Approach with Alignment Methods That Preserve Resolution. J Struct Biol 120:343-352.
Moon A F, et al. 2016. Structural characterization of the virulence factor Sda1 nuclease from Streptococcus pyogenes. Nucleic Acids Res 44:3946-3957.
Nguyen Ba A N, et al. 2009. NLStradamus: a simple Hidden Markov Model for nuclear localization signal prediction. BMC Bioinformatics 10:202.
Penz T, et al. 2010. The genome of the amoeba symbiont “Candidatus Amoebophilus asiaticus” encodes an afp-like prophage possibly used for protein secretion. Virulence 1:541-545.
Penz T, et al. 2012. Comparative Genomics Suggests an Independent Origin of Cytoplasmic Incompatibility in Cardinium hertigii. PLOS Genet 8:e1003012.
Pukatzki S, et al. 2007. Type VI secretion system translocates a phage tail spike-like protein into target cells where it cross-links actin 104:15508-15513.
Sana T G, Flaugnatti N, Lugo K A, Lam L H, Jacobson A, Baylot V, Durand E, Journet L, Cascales E, Monack D M. 2016. Salmonella Typhimurium utilizes a T6SS-mediated antibacterial weapon to establish in the host gut. Proc Natl Acad Sci 113:E5044-E5051.
Sarris P F, et al. 2014. A Phage Tail-Derived Element with Wide Distribution among Both Prokaryotic Domains: A Comparative Genomic and Phylogenetic Study. Genome Biol Evol 6:1739-1747.
Scholl D. 2017. Phage Tail-Like Bacteriocins. Annu Rev Virol 4:453-467.
Shikuma N J, et al. 2016. Stepwise metamorphosis of the tubeworm Hydroides elegans is mediated by a bacterial inducer and MAPK signaling. Proc Natl Acad Sci 113:10097-10102.
Shikuma N J, et al. 2014. Marine Tubeworm Metamorphosis Induced by Arrays of Bacterial Phage Tail-Like Structures. Science 343:529-534.
Suarez G, et al. 2010. A type VI secretion system effector protein, VgrG1, from Aeromonas hydrophila that induces host cell toxicity by ADP ribosylation of actin. J Bacteriol 192:155-168.
Taylor N M I, et al. 2018. Contractile injection systems of bacteriophages and related systems. Mol Microbiol 108:6-15.
Thurber, Rebecca., et al, 2009. Lab procedures to generate viral metagenomics 470-483.
Weiss G L, et al. 2017. Bacterial Protein Secretion Systems In: Journet L, Cascales E, editors. Bacterial Protein Secretion Systems. pp. 353-375.
Yang G, et al. 2006. Photorhabdus virulence cassettes confer injectable insecticidal activity against the wax moth. J Bacteriol 188:2254-2261.

### References—Example 2

Basler M, et al, 2012. Type VI secretion requires a dynamic contractile phage tail-like structure. Nature 483:182-186.
Böck D, et al. 2017. In situ architecture, function, and evolution of a contractile injection system. Science 357:713-717.
Carpizo-Ituarte E, Hadfield MG. 1998. Stimulation of Metamorphosis in the Polychaete Hydroides elegans Haswell (Serpulidae). Biol Bull 194:14-24.
Danev R, et al. 2014. Volta potential phase plate for in-focus phase contrast transmission electron microscopy. Proc Natl Acad Sci 111:15635-15640.
Dworjanyn S A, Pirozzi I. 2008. Induction of settlement in the sea urchin Tripneustes gratilla by macroalgae, biofilms and conspecifics: A role for bacteria? Aquaculture 274:268-274.
Hadfield M G. 2011. Biofilms and Marine Invertebrate Larvae: What Bacteria Produce That Larvae Use to Choose Settlement Sites. Ann Rev Mar Sci 3:453-470.
Hadfield MG, et al. 1994. Settlement preferences of the ubiquitous fouler Hydroides elegans Recent Developments in Biofouling Control. pp. 65-74.
Heymann J B, et al. 2013. Three-dimensional structure of the toxin-delivery particle antifeeding prophage of serratia entomophila. J Biol Chem 288:25276-25284.
Huang S Y, Hadfield M G. 2003. Composition and density of bacterial biofilms determine larval settlement of the polychaete Hydroides elegans. Mar Ecol Ser 260:161-172.
Huang Y, et al. 2012. Recruitment in the sea: Bacterial genes required for inducing larval settlement in a polychaete worm. Sci Rep 2.
Jiang F, et al. 2016. The Pseudomonas aeruginosa Type VI Secretion PGAP1-like Effector Induces Host Autophagy by Activating Endoplasmic Reticulum Stress. Cell Rep 16:1502-1509.

Karimova G, Ullmann A, Ladant D. 2000. A bacterial two-hybrid system that exploits a cAMP signaling cascade in Escherichia coli. Methods Enzymol 328:59-73.
Khandeparker L, et al. 2006. Relevance of biofilm bacteria in modulating the larval metamorphosis of Balanus amphitrite. FEMS Microbiol Ecol 58:425-438.
Kremer J R, et al. 1996. Computer Visualization of Three-Dimensional Image Data Using IMOD. J Struct Biol 116:71-76.
Ma A T, Mekalanos J J. 2010. In vivo actin cross-linking induced by Vibrio cholerae type VI secretion system is associated with intestinal inflammation. Proc Natl Acad Sci USA 107:4365-70.
Mastronarde D N. 2008. Correction for non-perpendicularity of beam and tilt axis in tomographic reconstructions with the IMOD package. J Microsc 230:212-217.
Mastronarde D N. 2005. Automated electron microscope tomography using robust prediction of specimen movements. J Struct Biol 152:36-51.
McFall-Ngai M, et al. 2013. Animals in a bacterial world, a new imperative for the life sciences. Proc Natl Acad Sci 110:3229-3236.
Miller J H. 1972. Experiments in molecular genetics. Cold Spring Harbor Laboratory.
Nedved B T, Hadfield M G. 2008. Hydroides elegans (Annelida: Polychaeta): A Model for Biofouling Research. Springer, Berlin, Heidelberg. pp. 1-15.
Pettersen E F, et al. 2004. UCSF Chimera - A visualization system for exploratory research and analysis. J Comput Chem 25:1605-1612.
Rocchi I, et al. 2019. Bacterial Phage Tail-like Structure Kills Eukaryotic Cells by Injecting a Nuclease Effector. bioRxiv 543298.
Sarris P F, et al. 2014. A Phage Tail-Derived Element with Wide Distribution among Both Prokaryotic Domains: A Comparative Genomic and Phylogenetic Study. Genome Biol Evol 6:1739-1747.
Schaefer J, et al. 2016. A data comparison between a traditional and the single-step β-galactosidase assay. Data Br 8:350-352.
Scholl D. 2017. Phage Tail-Like Bacteriocins. Annu Rev Virol 4:453-467.
Shikuma N J, et al. 2016. Stepwise metamorphosis of the tubeworm Hydroides elegans is mediated by a bacterial inducer and MAPK signaling. Proc Natl Acad Sci 113:10097-10102.
Shikuma N J, et al. 2014. Marine Tubeworm Metamorphosis Induced by Arrays of Bacterial Phage Tail-Like Structures. Science 343:529-534.
Shneider M M, et al. 2013. PAAR-repeat proteins sharpen and diversify the Type VI secretion system spike. Nature 500:350-353.
Silverman J M, et al. 2013. Haemolysin Co-regulated Protein is an Exported Receptor and Chaperone of Type VI Secretion Substrates. Mol Cell 51.
Sneed J M, Sharp K H, Ritchie K B, Paul V J. 2014. The chemical cue tetrabromopyrrole from a biofilm bacterium induces settlement of multiple Caribbean corals. Proc R Soc B Biol Sci 281:20133086-20133086.
Swanson R L, Marshall D J, Steinberg P D. 2007. Larval desperation and histamine: how simple responses can lead to complex changes in larval behaviour. J Exp Biol 210:3228-3235.
Taylor N M I, van Raaij M J, Leiman P G. 2018. Contractile injection systems of bacteriophages and related systems. Mol Microbiol 108:6-15.
Tebben J, Tapiolas D M, Motti C A, Abrego D, Negri A P, Blackall L L, Steinberg P D, Harder T. 2011. Induction of larval metamorphosis of the coral Acropora millepora by tetrabromopyrrole isolated from a Pseudoalteromonas bacterium. PLOS One 6:1-8.
Vlisidou I, et al. 2019. Photorhabdus Virulence Cassettes: extracellular multi-protein needle complexes for delivery of small protein effectors into host cells. bioRxiv 549964.
Webster N S, et al. 2004. Metamorphosis of a scleractinian coral in response to microbial biofilms. Appl Environ Microbiol 70:1213-21.
Weiss G L, Medeiros J M, Pilhofer M. 2017. In Situ Imaging of Bacterial Secretion Systems by Electron CryotomographyMethods in Molecular Biology (Clifton, N.J.). pp. 353-375.
Weiss S, et al. 2017. Normalization and microbial differential abundance strategies depend upon data characteristics. Microbiome 5:1-18.
Whalan S, Webster N S. 2014. Sponge larval settlement cues: The role of microbial biofilms in a warming ocean. Sci Rep 4:28-32.
Yool A J, et al. 1986. Excess Potassium Induces Larval Metamorphosis In Four Marine Invertebrate Species. Biol Bull 170:255-266.
Yu X, He W, Li H, Yan Y, Lin C. 2010. Larval settlement and metamorphosis of the pearl oyster Pinctada fucata in response to biofilms. Aquaculture 306:334-337.
Zeller R W. 2018. Electroporation in Ascidians: History, Theory and Protocols Transgenic Ascidians. Springer International Publishing AG. pp. 37-48.
Zeller R W, et al. 2006. Predictable mosaic transgene expression in ascidian embryos produced with a simple electroporation device. Dev Dyn 235:1921-1932.
Zobell C E, Allen E C. 1935. The Significance of Marine Bacteria in the Fouling of Submerged Surfaces. J Bacteriol 29:239-51.

### References Example 3

1 Gill, S. R. et al. Metagenomic Analysis of the Human Distal Gut Microbiome. 312, (2006).
2. Consortium, T. H. M. P. et al. Structure, function and diversity of the healthy human microbiome. Nature 486, 207 (2012).
3. Ley, R. E., Turnbaugh, P. J., Klein, S. & Gordon, J. I. Human gut microbes associated with obesity. Nature 444, 1022-1023 (2006).
4. Hooper, L. V. & Gordon, J. I. Commensal host-bacterial relationships in the gut. Science (80-.). 292, 1115-1118 (2001).
5. Kau, A. L., Ahern, P. P., Griffin, N. W., Goodman, A. L. & Gordon, J. I. Human nutrition, the gut microbiome and the immune system. Nature 474, 327 (2011).
6. Subramanian, S. et al. Persistent gut microbiota immaturity in malnourished Bangladeshi children. Nature 510, 417-421 (2014).

7. Carroll, I. M. et al. Molecular analysis of the luminal- and mucosal-associated intestinal microbiota in diarrhea-predominant irritable bowel syndrome. Am. J. Physiol.-Gastrointest. Liver Physiol. 301, 799-807 (2011).
8. Cianfanelli, F. R., Monlezun, L. & Coulthurst, S. J. Aim, Load, Fire: The Type VI Secretion System, a Bacterial Nanoweapon. Trends Microbiol. 24, 51-62 (2016).
9. Salmond, G. P. C. & Fineran, P. C. A century of the phage: past, present and future. Nat. Rev. Microbiol. 13, 777-86 (2015).
10. Russell, A. B. et al. A type VI secretion-related pathway in Bacteroidetes mediates interbacterial antagonism. Cell Host Microbe 16, 227-236 (2014).
11. Chatzidaki-Livanis, M., Geva-Zatorsky, N. & Comstock, L. E. Bacteroides fragilis type VI secretion systems use novel effector and immunity proteins to antagonize human gut Bacteroidales species. Proc. Natl. Acad. Sci. U.S.A. 113, 3627-32 (2016).
12. Verster, A. J. et al. The Landscape of Type VI Secretion across Human Gut Microbiomes Reveals Its Role in Community Composition. Cell Host Microbe 22, 411-419.e4 (2017).
13. Coyne, M. J., Roelofs, K. G. & Comstock, L. E. Type VI secretion systems of human gut Bacteroidales segregate into three genetic architectures, two of which are contained on mobile genetic elements. BMC Genomics 17, 1-21 (2016).
14. Böck, D. et al. In situ architecture, function, and evolution of a contractile injection system injection system. Scien 357, 713-717 (2017).
15. Shikuma, N. J. et al. Marine Tubeworm Metamorphosis Induced by Arrays of Bacterial Phage Tail-Like Structures. Science (80-.). 343, 529-533 (2014).
16. Shikuma, N. J., Antoshechkin, I., Medeiros, J. M., Pilhofer, M. & Newman, D. K. Stepwise metamorphosis of the tubeworm Hydroides elegans is mediated by a bacterial inducer and MAPK signaling. Proc. Natl. Acad. Sci. 113, 10097-10102 (2016).
17. Yang, G., Dowling, A. J., Gerike, U., Ffrench-Constant, R. H. & Waterfield, N. R. Photorhabdus Virulence Cassettes Confer Injectable Insecticidal Activity against the Wax Moth. J. Bacteriol. 188, 2254-2261 (2006).
18. Hurst, M. R. H., Beard, S. S., Jackson, T. A. & Jones, S. M. Isolation and characterization of the Serratia entomophila antifeeding prophage. FEMS Microbiol. Lett. 270, 42-48 (2007).
19. Heymann, J. B. et al. Three-dimensional structure of the toxin-delivery particle antifeeding prophage of Serratia entomophila. J. Biol. Chem. 288, 25276-25284 (2013).
20 Hurst, M. R. H. et al. Serratia proteamaculans Strain AGR96X Encodes an Antifeeding Prophage (Tailocin) with Activity against Grass Grub (Costelytra giveni) and Manuka Beetle (Pyronota Species) Larvae. Appl. Environ. Microbiol. 84, e02739-17 (2018).
21. Hurst, M. R. H., Glare, T. R. & Jackson, T. A. Cloning Serratia entomophila antifeeding genes—A putative defective prophage active against the grass grub Costelytra zealandica. J. Bacteriol. 186, 7023-7024 (2004).
22 Sarris, P. F., Ladoukakis, E. D., Panopoulos, N. J. & Scoulica, E. V. A phage tail-derived element with wide distribution among both prokaryotic domains: A comparative genomic and phylogenetic study. Genome Biol. Evol. 6, 1739-1747 (2014).
23. Penz, T. et al. Comparative Genomics Suggests an Independent Origin of Cytoplasmic Incompatibility in Cardinium hertigii. PLOS Genet. 8, (2012).
24. McNulty, N. P. et al. Effects of Diet on Resource Utilization by a Model Human Gut Microbiota Containing Bacteroides cellulosilyticus WH2, a Symbiont with an Extensive Glycobiome. PLOS Biol. 11, (2013).
25. Levi, K., Abeysinghe, E., Rynge, M. & Edwards, R. A. Searching the sequence read archive using Jetstream and Wrangler. ACM Int. Conf. Proceeding Ser. 1-7 (2018). doi: 10.1145/3219104.3229278
26. Ridaura, V. K. et al. Gut microbiota from twins discordant for obesity modulate metabolism in mice. Science 341, 1241214 (2013).
27 Myslicki, J. P., Belke, D. D. & Shearer, J. Role of O-GlcNAcylation in nutritional sensing, insulin resistance and in mediating the benefits of exercise. Appl. Physiol. Nutr. Metab. 39, 1205-1213 (2014).
28 Baudoin, L. & Issad, T. O-GlcNAcylation and inflammation: A vast territory to explore. Front. Endocrinol. (Lausanne). 5, 1-8 (2014).
29. Qin, J. et al. A human gut microbial gene catalogue established by metagenomic sequencing. Nature 464, 59-65 (2010).
30. Vlisidou, I. et al. Photorhabdus Virulence Cassettes: extracellular multi-protein needle complexes for delivery of small protein effectors into host cells. bioRxiv 549964 (2019). doi: 10.1101/549964
31. Ericson, C. et al. A contractile injection system stimulates tubeworm metamorphosis by translocating a proteinaceous effector. Elife 1-19 (2019). doi:https://doi.org/10.7554/eLife.46845
32. Marcobal, A. et al. Bacteroides in the infant gut consume milk oligosaccharides via mucus-utilization pathways. Cell Host Microbe 10, 507-514 (2011).
33 Tropini, C., Earle, K. A., Huang, K. C. & Sonnenburg, J. L. The Gut Microbiome: Connecting Spatial Organization to Function. Cell Host Microbe 21, 433-442 (2017).
34 Van Der Waaij, L. A. et al. Bacterial population analysis of human colon and terminal ileum biopsies with 16S rRNA-based fluorescent probes: Commensal bacteria live in suspension and have no direct contact with epithelial cells. Inflamm. Bowel Dis. 11, 865-871 (2005).
35 Rocchi, I. et al. Bacterial Phage Tail-like Structure Kills Eukaryotic Cells by Injecting a Nuclease Effector. bioRxiv 28, 543298 (2019).
36. Gouy, M., Guindon, S. & Gascuel, O. Sea view version 4: A multiplatform graphical user interface for sequence alignment and phylogenetic tree building. Mol. Biol. Evol. 27, 221-224 (2010).
37 Guindon, S. et al. New algorithms and methods to estimate maximum-likelihood phylogenies: Assessing the performance of PhyML 3.0. Syst. Biol. 59, 307-321 (2010).
38. Lefort, V., Longueville, J. E. & Gascuel, O. SMS: Smart Model Selection in PhyML. Mol. Biol. Evol. 34, 2422-2424 (2017).
39. Letunic, I. & Bork, P. Interactive tree of life (iTOL) v3: an online tool for the display and annotation of phylogenetic and other trees. Nucleic Acids Res. 44, W242-W245 (2016).
40. El-Gebali, S. et al. The Pfam protein families database in 2019. Nucleic Acids Res. 47, D427-D432 (2019).
41. Edgar, R. C. Search and clustering orders of magnitude faster than BLAST. Bioinformatics 26, 2460-2461 (2010).
42. Marchler-Bauer, A. & Bryant, S. H. CD-Search: Protein domain annotations on the fly. Nucleic Acids Res. 32, 327-331 (2004).
43. Marchler-Bauer, A. et al. CDD: A Conserved Domain Database for the functional annotation of proteins. Nucleic Acids Res. 39, 225-229 (2011).
44. Marchler-Bauer, A. et al. CDD/SPARCLE: Functional classification of proteins via subfamily domain architectures. Nucleic Acids Res. 45, D200-D203 (2017).
45. Remmert, M., Biegert, A., Hauser, A. & Söding, J. HHblits: Lightning-fast iterative protein sequence searching by HMM-HMM alignment. Nat. Methods 9, 173-175 (2012).
46. McGarvey, P. B. et al. UniProt genomic mapping for deciphering functional effects of missense variants. Hum. Mutat. 40, 694-705 (2019).
47. Shen, W., Le, S., Li, Y. & Hu, F. SeqKit: A cross-platform and ultrafast toolkit for FASTA/Q file manipulation. PLOS One 11, 1-10 (2016).
48. Hadley, W. ggplot2: Elegant Graphics for Data Analysis. (Springer, 2016). doi: 10.1002/wics.147
49 Csardi, G. & Nepusz, T. The igraph software package for complex network research. InterJournal (2006).

A number of embodiments of the invention have been described. Nevertheless, it will be understood that various modifications may be made without departing from the spirit and scope of the invention. Accordingly, other embodiments are within the scope of the following claims.

1: A recombinant bacterial Contractile Injection Systems (CIS) or a Metamorphosis Associated Contractile structure (MACs),
wherein the recombinant bacterial CIS or MACs comprise or have contained therein a proteinaceous cargo, or a heterologous protein or peptide,
and the proteinaceous cargo, or a heterologous protein or peptide comprises a Metamorphosis-Inducing Factor 1 (Mif1) protein, or is linked to a Mif1 (optionally is chemically linked or electrostatically linked), or is a fusion or a recombinant protein comprising a Mif1,
wherein the Mif1 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO: 1, or between about 80% to 100% sequence identity to SEQ ID NO: 1,
or the Mif1 protein comprises a sequence having at least about 85%, 86%, 87%, 88%, 89%, 90%, 91%, 92%, 93%, 94%, 95%, 96%, 97%, 98% or 100% sequence identity to SEQ ID NO:2, or between about 80% to 100% sequence identity to SEQ ID NO:2.

2: A liposome or lipid-comprising nanoparticle incorporating or expressing on its outer surface a substantially purified or isolated bacterial CIS or MACs, or a recombinant bacterial CIS or MACs,
wherein the substantially purified or isolated bacterial CIS, or the recombinant bacterial CIS or MACs, comprise or have contained therein a proteinaceous cargo, or a heterologous protein or peptide,
and the proteinaceous cargo, or a heterologous protein or peptide comprises a Metamorphosis-Inducing Factor 1 (Mif1) protein, or is linked to a Mif1 (optionally is chemically linked or electrostatically linked), or is a fusion or a recombinant protein comprising a Mif1,
wherein the Mif1 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO: 1, or between about 80% to 100% sequence identity to SEQ ID NO: 1,
or the Mif1 protein comprises a sequence having at least about 85%, 86%, 87%, 88%, 89%, 90%, 91%, 92%, 93%, 94%, 95%, 96%, 97%, 98% or 100% sequence identity to SEQ ID NO:2, or between about 80% to 100% sequence identity to SEQ ID NO:2.

3: A protoplast or a spheroplast incorporating or expressing on its outer surface a substantially purified or isolated bacterial CIS or MACs, or a recombinant bacterial CIS or MACs, of claim 1.

4: A cell expressing on its extracellular surface a substantially purified or isolated bacterial CIS or MACs, or a recombinant bacterial CIS or MACs, of claim 1
wherein the CIS or MACs are heterologous to the cell,
wherein the cell is a microbial cell or a eukaryotic cell, and optionally the microbial cell is a bacterial cell.

5: A method for delivering a proteinaceous cargo, or a protein or a peptide, or a drug or a marker, to a cell, optionally to a eukaryotic, mammalian or human cell, or to a plant cell, or to an individual in need thereof, comprising:
(a) providing a formulation or composition comprising:
(i) a substantially purified or isolated bacterial Contractile Injection Systems (CIS) or Metamorphosis Associated Contractile structure (MACs) of claim 1;
(ii) a recombinant bacterial Contractile Injection Systems (CIS) or Metamorphosis Associated Contractile structure (MACs) of claim 1;
(iii) a liposome or lipid-comprising nanoparticle incorporating or expressing on its outer surface the substantially purified or isolated bacterial CIS or MACs, or the recombinant bacterial CIS or MACs of claim 1;
(iv) a protoplast or a spheroplast incorporating or expressing on its outer surface the substantially purified or isolated bacterial CIS or MACs, or the recombinant bacterial CIS or MACs of claim 1;
(v) a cell expressing on its extracellular surface the substantially purified or isolated bacterial CIS or MACs, or the recombinant bacterial CIS or MACs of claim 1; or
(vi) any combination of (i) to (v);
(b) contacting the formulation or composition with the cell under conditions wherein the formulation or composition, or the proteinaceous cargo, or the protein or peptide, contacts or interacts with the cell, thereby delivering the proteinaceous cargo, or the protein or peptide to or into the cell.

6: The method of claim 5, wherein the proteinaceous cargo, or the protein or peptide, comprises or is an antibody, an enzyme, a drug, a marker, a detectable moiety, or an active biological agent.

7: The method of claim 5, wherein the contacting of the formulation or composition with the cell eukaryotic cell is in vitro, ex vivo, or in vivo.

8: The method of claim 5, wherein the eukaryotic cell is a mammalian, human or an animal cell.

8: A kit comprising: a formulation or composition comprising:
(i) a substantially purified or isolated bacterial Contractile Injection Systems (CIS) or Metamorphosis Associated Contractile structure (MACs) of claim 1;
(ii) a recombinant bacterial Contractile Injection Systems (CIS) or Metamorphosis Associated Contractile structure (MACs) of claim 1;
(iii) a liposome or lipid-comprising nanoparticle incorporating or expressing on its outer surface the substantially purified or isolated bacterial CIS or MACs, or the recombinant bacterial CIS or MACs, of claim 1;
(iv) a protoplast or a spheroplast incorporating or expressing on its outer surface the substantially purified or isolated bacterial CIS or MACs, or the recombinant bacterial CIS or MACs, of claim 1;
(v) a cell expressing on its extracellular surface the substantially purified or isolated bacterial CIS or MACs, or the recombinant bacterial CIS or MACs, of claim 1,
wherein optionally the cell is a microbial cell or a eukaryotic cell, or a mammalian or a human cell, and optionally the microbial cell is a bacterial cell; or
(vi) any combination of (i) to (v).

10-11. (canceled)

12: The method of claim 5, wherein the cell is a microbial cell or a eukaryotic cell.

13: The method of claim 12, wherein the eukaryotic cell is a mammalian or a human cell, or the microbial cell is a bacterial cell


SEQUENCE LISTING (111 sequences)

SEQ ID NO: 1 - DNA, 2832 bp, Artificial Sequence
  Synthetic
atgcaacaacaagaacaggagcaagctcctacctttcaaagttatcccacgcgcactgcgttatcagtgagcaatgcattagactctgattctgtgagtgtcgatctactctcatcgggcgttgatgttgcaaagcggtatgcctcacaagtggtttgggatcattttaatggcgatgcaacagctaagctgattatctcaagtgtatttaattatgcggtgacatcattaaagtccttgaacccatgggtgacggcaatctcacaagcacttttacttttggcaaaagttccgcctggtgttgtttccgccgttttatgggccattggaaaaatctggctttgggctgcaaataaattttataacggtggttggatagccgctgcgtggggagatattgatgagccatatatttatcaatggttaaaaaaaggcagtgatgcacatggggcattacgggcactcgtggatgatttaaaagcttgggttaagtatattcaagataagcttgccagtagcgttgcacgtttaattggtgtctcggattcaagtagtgaagatgagcaaagcgatgagcaacaaacggatcaagatgcacaaacatcacctaatatagttgataataagttcatttcgttggggataaaccaacctgaattattggactgggaagagggggcaagtaagcccaagcgcgctggactacatggtacgggtgttgccaaggtcaatctgcttggtcagcaatttggtggtgatatagatgtaaaattaccatttggcagtggctgggaagtggctgtatctccggtttttgcaagccaacaaggggtgggttttagtggttatattgaagcgcatcaagtattgggcgatgagctgaccattaatgaagaaggccttgctcgttttaaagcaagcattgttgggctaaacattgcgaacaagcgggtttactccgatttaatgtcacttgattacaataagcagcaaaaagaagttcattttgcaggtgatgcacatgtgccgctttgggataaaaagaaactggatggtgagtttgatttaaaattggatactgcaggtaaattcaaatcaggcaggaccaaaatctcttctaaagatacattcgaagtaatccccaagtttttgtctatcagcaacccgagtggtgaagtagaagttaaagaggatgcttcaccagagtttgaggtgagtactgacgctgcgctttacggcttacccgctggtgtgaaagcatcaattacgcaagcaaaagtgatgtatcacgatgagcaattaggcggtgaggttgagcaggctgatgtggagatcccaattagcaaacacactacaatgacgctggccatgacgaaggctaagtttacaaaagatgcaattgaagctgaaaatgcttttatggatctcgatcatgataatgccaaagtgcttaatgaaacggccattgtcgagtcagactttttctctggaaacttcgattttggaaaagtgtttgagcttaagcagttgaaagtccaccaagggttaaacgcgcttaaatttgctggtggtaaattctcctacgagaaggatagcaacaatggtttacagttacttagagcacgtattttaggccttgaggcttactataatagaaaagatcgtgaaggtgggatcactggagaatggcacaaagggattgattttccggctttctctttggactttcccgttgcggctggtgttgcaggagttggagttgatattaccggggggtttgagtttggtgcgagcatcggcgctaagcttaaaaatgaagagcagcataacacagagacacaaatgcttttctctgtgaatggtgcagcttcggccagtgccaaagctaaagcgagaattgaggctggtgcttttgtgggggtcccttatcttgcaaaggttcaaggtggtgtatttggtgagatccgaggtggtgttgaaggtaaggttgaaacagaagggcgtttgaaatacacgcgtggcaaaggcggtgatagctttggtcaatttgccattgatagtgattatccaatggaggcgagctttagcctaaccggctcattagaagcggaagtaggtgcatcgataaaagccaaagtactgacatttgaaaaagaaattgcgtcggtatctattggtgattggacactgggtgagtatacgcttaatggtaaaatcaaaaaagatcctaatgggaaagggtatatcgttgagcgctctaaaggggagtttaccgaaggcaaaccaaaacctgccgaggtaactaaggaggcactcccagtagataagtgggttaatcagttagaaaaagatcatactcatattgaatacaccgaagatgaagccaaaagaaagttacctgaggttgcacgtaaaacggtatatagtcacctgagtccaatgcaaaaagaggatgtaagtgagcgatatactcatttgctcaagttgatatcgcagcaaaataagttcactgaaatttatcgagaaaccaaagctgatagggataagttaccgaccagtggcacttatatttggacctctaaagtttggaatgaagcggtacagcgcaagagtttctggatttttgaaatgagtaagagtaaagcaaaagtggcggataagctggataaatatcacaaaacgcactctattttagagcggagactgttacttgcaaagctagaagagatagcctcggactatcgcaaaaaccgctcgaaaaacgcagaaaataaagaagctgccggggagtttttagagagtattgaaaaagaaaggctcattttaatgtaa

SEQ ID NO: 2 - protein, 943 aa, Artificial Sequence
  Synthetic
MQQQEQEQAPTFQSYPTRTALSVSNALDSDSVSVDLLSSGVDVAKRYASQVVWDHFNGDATAKLIISSVFNYAVTSLKSLNPWVTAISQALLLLAKVPPGVVSAVLWAIGKIWLWAANKFYNGGWIAAAWGDIDEPYIYQWLKKGSDAHGALRALVDDLKAWVKYIQDKLASSVARLIGVSDSSSEDEQSDEQQTDQDAQTSPNIVDNKFISLGINQPELLDWEEGASKPKRAGLHGTGVAKVNLLGQQFGGDIDVKLPFGSGWEVAVSPVFASQQGVGFSGYIEAHQVLGDELTINEEGLARFKASIVGLNIANKRVYSDLMSLDYNKQQKEVHFAGDAHVPLWDKKKLDGEFDLKLDTAGKFKSGRTKISSKDTFEVIPKFLSISNPSGEVEVKEDASPEFEVSTDAALYGLPAGVKASITQAKVMYHDEQLGGEVEQADVEIPISKHTTMTLAMTKAKFTKDAIEAENAFMDLDHDNAKVLNETAIVESDFFSGNFDFGKVFELKQLKVHQGLNALKFAGGKFSYEKDSNNGLQLLRARILGLEAYYNRKDREGGITGEWHKGIDFPAFSLDFPVAAGVAGVGVDITGGFEFGASIGAKLKNEEQHNTETQMLFSVNGAASASAKAKARIEAGAFVGVPYLAKVQGGVFGEIRGGVEGKVETEGRLKYTRGKGGDSFGQFAIDSDYPMEASFSLTGSLEAEVGASIKAKVLTFEKEIASVSIGDWTLGEYTLNGKIKKDPNGKGYIVERSKGEFTEGKPKPAEVTKEALPVDKWVNQLEKDHTHIEYTEDEAKRKLPEVARKTVYSHLSPMQKEDVSERYTHLLKLISQQNKFTEIYRETKADRDKLPTSGTYIWTSKVWNEAVQRKSFWIFEMSKSKAKVADKLDKYHKTHSILERRLLLAKLEEIASDYRKNRSKNAENKEAAGEFLESIEKERLILM

SEQ ID NO: 3 - DNA, 60 bp, Artificial Sequence
  Synthetic: macB_dA
ggtcgacggatcccaagcttcttctagaggtaccgcatgcaacccagacactgaggtgct

SEQ ID NO: 4 - DNA, 46 bp, Artificial Sequence
  Synthetic: macB_dB
tttccattttccaatcccttcgccagagataagtgattgactacga

SEQ ID NO: 5 - DNA, 46 bp, Artificial Sequence
  Synthetic: macB_dC
tcgtagtcaatcacttatctctggcgaagggattggaaaatggaaa

SEQ ID NO: 6 - DNA, 60 bp, Artificial Sequence
  Synthetic: macB_dD
acacaacgtgaattcaaagggagagctcgatatcgcatgccataacctggctgagcacct

SEQ ID NO: 7 - DNA, 50 bp, Artificial Sequence
  Synthetic: 1556_dA
tgatgggttaaaaaggatcgatcctctagattggagcaataaacgggttc

SEQ ID NO: 8 - DNA, 44 bp, Artificial Sequence
  Synthetic: 1556_dB
gttcataattaaactgcgatcgcagccataaggcctccttgata

SEQ ID NO: 9 - DNA, 44 bp, Artificial Sequence
  Synthetic: 1556_dC
tatcaaggaggccttatggctgcgatcgcagtttaattatgaac

SEQ ID NO: 10 - DNA, 55 bp, Artificial Sequence
  Synthetic: 1556_dD
ttttgagacacaacgtgaattcaaagggagagctccgctttgggtactggcttta

SEQ ID NO: 11 - DNA, 20 bp, Artificial Sequence
  Synthetic: 1556_intF
ccgagcaaacgttatcacaa

SEQ ID NO: 12 - DNA, 20 bp, Artificial Sequence
  Synthetic: 1556_intR
tcagcgctctcattatgtgc

SEQ ID NO: 13 - DNA, 50 bp, Artificial Sequence
  Synthetic: 1555_dA
tgatgggttaaaaaggatcgatcctctagaccgagcaaacgttatcacaa

SEQ ID NO: 14 - DNA, 45 bp, Artificial Sequence
  Synthetic: 1555_dB
ccttgcatgaggttaagaaagtttgacgtacccttcagccatatt

SEQ ID NO: 15 - DNA, 45 bp, Artificial Sequence
  Synthetic: 1555_dC
aatatggctgaagggtacgtcaaactttcttaacctcatgcaagg

SEQ ID NO: 16 - DNA, 55 bp, Artificial Sequence
  Synthetic: 1555_dD
ttttgagacacaacgtgaattcaaagggagagctcgatgcggtaacggttgttct

SEQ ID NO: 17 - DNA, 20 bp, Artificial Sequence
  Synthetic: 1555_intF
agcgattgatgctgaacaaa

SEQ ID NO: 18 - DNA, 20 bp, Artificial Sequence
  Synthetic: 1555_intR
accatcgcataacccgtaac

SEQ ID NO: 19 - DNA, 50 bp, Artificial Sequence
  Synthetic: 1554_dA
tgatgggttaaaaaggatcgatcctctagatacgccgtccagttaggact

SEQ ID NO: 20 - DNA, 41 bp, Artificial Sequence
  Synthetic: 1554_dB
gtttgttaacgtcacggcagctgcattgccatttaaactcc

SEQ ID NO: 21 - DNA, 41 bp, Artificial Sequence
  Synthetic: 1554_dC
ggagtttaaatggcaatgcagctgccgtgacgttaacaaac

SEQ ID NO: 22 - DNA, 55 bp, Artificial Sequence
  Synthetic: 1554_dD
ttttgagacacaacgtgaattcaaagggagagctcattgattggaagcgcgatag

SEQ ID NO: 23 - DNA, 20 bp, Artificial Sequence
  Synthetic: 1554_intF
tttatgaggcaccaacgaca

SEQ ID NO: 24 - DNA, 20 bp, Artificial Sequence
  Synthetic: 1554_intR
gcctgtgccgttttatctgt

SEQ ID NO: 25 - DNA, 50 bp, Artificial Sequence
  Synthetic: 1553_dA
tgatgggttaaaaaggatcgatcctctagaggcgatcagtggagtgaagt

SEQ ID NO: 26 - DNA, 40 bp, Artificial Sequence
  Synthetic: 1553_dC
acatgacagaagaagcacgcggggctgagcaagaagtatt

SEQ ID NO: 27 - DNA, 55 bp, Artificial Sequence
  Synthetic: 1553_dD
ttttgagacacaacgtgaattcaaagggagagctctcagaaccagcagtctcacg

SEQ ID NO: 28 - DNA, 20 bp, Artificial Sequence
  Synthetic: 1553_intF
cgggcctagaaatcactcaa

SEQ ID NO: 29 - DNA, 20 bp, Artificial Sequence
  Synthetic: 1553_intR
tcgacgtcaaatcagtcgag

SEQ ID NO: 30 - DNA, 50 bp, Artificial Sequence
  Synthetic: 1552_dA
tgatgggttaaaaaggatcgatcctctagagagagcaagaagtggcgagt

SEQ ID NO: 31 - DNA, 40 bp, Artificial Sequence
  Synthetic: 1552_dB
tagccttttagtgccgcttttgaggcgtccatatctgaca

SEQ ID NO: 32 - DNA, 40 bp, Artificial Sequence
  Synthetic: 1552_dC
tgtcagatatggacgcctcaaaagcggcactaaaaggcta

SEQ ID NO: 33 - DNA, 55 bp, Artificial Sequence
  Synthetic: 1552_dD
ttttgagacacaacgtgaattcaaagggagagctctgctgaccaagcagattgac

SEQ ID NO: 34 - DNA, 20 bp, Artificial Sequence
  Synthetic: 1552_intF
gggcaattgttgtggatttt

SEQ ID NO: 35 - DNA, 20 bp, Artificial Sequence
  Synthetic: 1552_intR
tgatcccaaaccacttgtga

SEQ ID NO: 36 - DNA, 51 bp, Artificial Sequence
  Synthetic: 1551_dA
tgatgggttaaaaaggatcgatcctctagagactgctggttctgattcgat

SEQ ID NO: 37 - DNA, 49 bp, Artificial Sequence
  Synthetic: 1551_dB
aacagatcattacattaaaatgagcctctgttcttgttgttgcatttca

SEQ ID NO: 38 - DNA, 49 bp, Artificial Sequence
  Synthetic: 1551_dC
tgaaatgcaacaacaagaacagaggctcattttaatgtaatgatctgtt

SEQ ID NO: 39 - DNA, 55 bp, Artificial Sequence
  Synthetic: 1551_dD
ttttgagacacaacgtgaattcaaagggagagctccttctccattttcgcctttg

SEQ ID NO: 40 - DNA, 20 bp, Artificial Sequence
  Synthetic: 1551_intF
cgttttcagtgaccatcacg

SEQ ID NO: 41 - DNA, 20 bp, Artificial Sequence
  Synthetic: 1551_intR
cggtgggcaaaaaggtataa

SEQ ID NO: 42 - DNA, 45 bp, Artificial Sequence
  Synthetic: pET15b_12610_F1
cctggtgccgcgcggcagccatatgatgatgtcagatatggacgc

SEQ ID NO: 43 - DNA, 45 bp, Artificial Sequence
  Synthetic: pET15b_12610_R1
tcgggctttgttagcagccggatccttagccttttagtgccgctt

SEQ ID NO: 44 - DNA, 40 bp, Artificial Sequence
  Synthetic: 12610--delta-NLS_C
atagcgaagagcagaagcaacaaaactcagatatgagtat

SEQ ID NO: 45 - DNA, 20 bp, Artificial Sequence
  Synthetic: JF50_12610_SeqF1
cctgaagggtcgttttcagt

SEQ ID NO: 46 - DNA, 21 bp, Artificial Sequence
  Synthetic: JF50_12610_E328A_F
gctcgagttgcgtctcatgta

SEQ ID NO: 47 - DNA, 21 bp, Artificial Sequence
  Synthetic: JF50_12610_E328A_R
tacatgagacgcaactcgagc

SEQ ID NO: 48 - protein, 15 aa, Artificial Sequence
  Synthetic: peptide
CERSKGEFTEGKPKP

SEQ ID NO: 49 - DNA, 50 bp, Artificial Sequence; identical to SEQ ID NO: 7
  Synthetic: 1556_dA

SEQ ID NO: 50 - DNA, 44 bp, Artificial Sequence; identical to SEQ ID NO: 8
  Synthetic: 1556_dB

SEQ ID NO: 51 - DNA, 44 bp, Artificial Sequence; identical to SEQ ID NO: 9
  Synthetic: 1556_dC

SEQ ID NO: 52 - DNA, 55 bp, Artificial Sequence; identical to SEQ ID NO: 10
  Synthetic: 1556_dD

SEQ ID NO: 53 - DNA, 20 bp, Artificial Sequence
  Synthetic: 1556_intF
ccgagcaaacgttatcacaa

SEQ ID NO: 54 - DNA, 20 bp, Artificial Sequence
  Synthetic: 1556_intR
tcagcgctctcattatgtgc

SEQ ID NO: 55 - DNA, 50 bp, Artificial Sequence; identical to SEQ ID NO: 13
  Synthetic: 1555_dA

SEQ ID NO: 56 - DNA, 45 bp, Artificial Sequence; identical to SEQ ID NO: 14
  Synthetic: 1555_dB

SEQ ID NO: 57 - DNA, 45 bp, Artificial Sequence; identical to SEQ ID NO: 15
  Synthetic: 1555_dC

SEQ ID NO: 58 - DNA, 55 bp, Artificial Sequence; identical to SEQ ID NO: 16
  Synthetic: 1555_dD

SEQ ID NO: 59 - DNA, 20 bp, Artificial Sequence
  Synthetic: 1555_intF
agcgattgatgctgaacaaa

SEQ ID NO: 60 - DNA, 20 bp, Artificial Sequence
  Synthetic: 1555_intR
accatcgcataacccgtaac

SEQ ID NO: 61 - DNA, 50 bp, Artificial Sequence; identical to SEQ ID NO: 19
  Synthetic: 1554_dA

SEQ ID NO: 62 - DNA, 41 bp, Artificial Sequence; identical to SEQ ID NO: 20
  Synthetic: 1554_dB

SEQ ID NO: 63 - DNA, 41 bp, Artificial Sequence; identical to SEQ ID NO: 21
  Synthetic: 1554_dC

SEQ ID NO: 64 - DNA, 55 bp, Artificial Sequence; identical to SEQ ID NO: 22
  Synthetic: 1554_dD

SEQ ID NO: 65 - DNA, 20 bp, Artificial Sequence
  Synthetic: 1554_intF
tttatgaggcaccaacgaca

SEQ ID NO: 66 - DNA, 20 bp, Artificial Sequence
  Synthetic: 1554_intR
gcctgtgccgttttatctgt

SEQ ID NO: 67 - DNA, 50 bp, Artificial Sequence; identical to SEQ ID NO: 25
  Synthetic: 1553_dA

SEQ ID NO: 68 - DNA, 40 bp, Artificial Sequence
  Synthetic: 1553_dB
aatacttcttgctcagccccgcgtgcttcttctgtcatgt

SEQ ID NO: 69 - DNA, 40 bp, Artificial Sequence; identical to SEQ ID NO: 26
  Synthetic: 1553_dC

SEQ ID NO: 70 - DNA, 55 bp, Artificial Sequence; identical to SEQ ID NO: 27
  Synthetic: 1553_dD

SEQ ID NO: 71 - DNA, 20 bp, Artificial Sequence
  Synthetic: 1553_intF
cgggcctagaaatcactcaa

SEQ ID NO: 72 - DNA, 20 bp, Artificial Sequence
  Synthetic: 1553_intR
tcgacgtcaaatcagtcgag

SEQ ID NO: 73 - DNA, 50 bp, Artificial Sequence; identical to SEQ ID NO: 30
  Synthetic: 1552_dA

SEQ ID NO: 74 - DNA, 40 bp, Artificial Sequence
  Synthetic: 12610--delta-NLS_B
atactcatatctgagttttgttgcttctgctcttcgctat

SEQ ID NO: 75 - DNA, 40 bp, Artificial Sequence; identical to SEQ ID NO: 32
  Synthetic: 1552_dC

SEQ ID NO: 76 - DNA, 55 bp, Artificial Sequence; identical to SEQ ID NO: 33
  Synthetic: 1552_dD

SEQ ID NO: 77 - DNA, 20 bp, Artificial Sequence
  Synthetic: 1552_intF
gggcaattgttgtggatttt

SEQ ID NO: 78 - DNA, 39 bp, Artificial Sequence
  Synthetic: 1551_dA
tgatgggttaaaaaggatcgatcctctagagactgctgg

SEQ ID NO: 79 - DNA, 12 bp, Artificial Sequence
  Synthetic: 1551_dA
ttctgattcgat

SEQ ID NO: 80 - DNA, 49 bp, Artificial Sequence; identical to SEQ ID NO: 37
  Synthetic: 1551_dB

SEQ ID NO: 81 - DNA, 49 bp, Artificial Sequence; identical to SEQ ID NO: 38
  Synthetic: 1551_dC

SEQ ID NO: 82 - DNA, 55 bp, Artificial Sequence; identical to SEQ ID NO: 39
  Synthetic: 1551_dD

SEQ ID NO: 83 - DNA, 20 bp, Artificial Sequence
  Synthetic: 1551_intF
cgttttcagtgaccatcacg

SEQ ID NO: 84 - DNA, 20 bp, Artificial Sequence
  Synthetic: 1551_intR
cggtgggcaaaaaggtataa

SEQ ID NO: 85 - DNA, 60 bp, Artificial Sequence
  Synthetic: pUT18_605_F1
cagctatgaccatgattacgccaagcttgcatgccatgacagaagaagcacgcgaaaaaa

SEQ ID NO: 86 - DNA, 60 bp, Artificial Sequence
  Synthetic: pUT18_605_R1
ctggcggctgaattcgagctcggtacccggggatcattcacaagtgctaattgataaaat

SEQ ID NO: 87 - DNA, 60 bp, Artificial Sequence
  Synthetic: pUT18_615_F1
cagctatgaccatgattacgccaagcttgcatgccatgcaacaacaagaacaggagcaag

SEQ ID NO: 88 - DNA, 60 bp, Artificial Sequence
  Synthetic: pUT18_615_R1
ctggcggctgaattcgagctcggtacccggggatccattaaaatgagcctttctttttca

SEQ ID NO: 89 - DNA, 50 bp, Artificial Sequence
  Synthetic: pUT18_680_F
catgattacgccaagcttgcatgccatggctactactaaagcagatatcg

SEQ ID NO: 90 - DNA, 50 bp, Artificial Sequence
  Synthetic: pUT18_680_R
aattcgagctcggtacccggggatcatggaactcaatcttgatgtcatct

SEQ ID NO: 91 - DNA, 60 bp, Artificial Sequence
  Synthetic: pKT_605_F1
ccgattacctggcgcgcacgcggcgggctgcagggatgacagaagaagcacgcgaaaaaa

SEQ ID NO: 92 - DNA, 60 bp, Artificial Sequence
  Synthetic: pKT_605_R1
aacgacggccgaattcttagttacttaggtacccgctaattcacaagtgctaattgataa

SEQ ID NO: 93 - DNA, 60 bp, Artificial Sequence
  Synthetic: pKT_615_F1
ccgattacctggcgcgcacgcggcgggctgcagggatgcaacaacaagaacaggagcaag

SEQ ID NO: 94 - DNA, 60 bp, Artificial Sequence
  Synthetic: pKT_615_R1
aacgacggccgaattcttagttacttaggtacccgttacattaaaatgagcctttctttt

SEQ ID NO: 95 - DNA, 50 bp, Artificial Sequence
  Synthetic: pKT_680_F1
cctggcgcgcacgcggcgggctgcaatggctactactaaagcagatatcg

SEQ ID NO: 96 - skipped

SEQ ID NO: 97 - DNA, 50 bp, Artificial Sequence
  Synthetic: pKT_680_R1
gccgaattcttagttacttaggtacttaatggaactcaatcttgatgtca

SEQ ID NO: 98 - DNA, 60 bp, Artificial Sequence; identical to SEQ ID NO: 85
  Synthetic: pKNT_605_F1

SEQ ID NO: 99 - DNA, 60 bp, Artificial Sequence
  Synthetic: pKNT_605_R1
tgatgcgattgctgcatggtcattgaattcgagctattcacaagtgctaattgataaaat

SEQ ID NO: 100 - DNA, 60 bp, Artificial Sequence; identical to SEQ ID NO: 87
  Synthetic: pKNT_615_F1

SEQ ID NO: 101 - DNA, 60 bp, Artificial Sequence
  Synthetic: pKNT_615_R1
tgatgcgattgctgcatggtcattgaattcgagctcattaaaatgagcctttctttttca

SEQ ID NO: 102 - DNA, 50 bp, Artificial Sequence
  Synthetic: pKNT_615_F2
catgattacgccaagcttgcatgccatgcaacaacaagaacaggagcaag

SEQ ID NO: 103 - DNA, 50 bp, Artificial Sequence; identical to SEQ ID NO: 89
  Synthetic: pKNT25-680_F

SEQ ID NO: 104 - DNA, 50 bp, Artificial Sequence
  Synthetic: pKNT25-680_R
gctgcatggtcattgaattcgagctatggaactcaatcttgatgtcatct

SEQ ID NO: 105 - DNA, 39 bp, Artificial Sequence
  Synthetic: pET15b_605_F1
tgccgcgcggcagccatatgatgacagaagaagcacgcg

SEQ ID NO: 106 - DNA, 41 bp, Artificial Sequence
  Synthetic: pET15b_605_R1
gctttgttagcagccggatccctaattcacaagtgctaatt

SEQ ID NO: 107 - protein, 91 aa, Pseudoalteromonas luteoviolacea
  misc_feature: Pne1
LAPRGESAEVNADQSDLMKNKLVYNNNGNNANEKGWIRGHLLNDNLGGSALKFNLYPITGSANKEHHARVESHVKNLVEAGYVVEYKVEVV

SEQ ID NO: 108 - protein, 107 aa, Streptococcus pyogenes
  misc_feature: spd1
LPFTANYQLQLGELDNLNRATFSHIQLQDRHETSSWVMNRGHLVGYQFCGLNDEPRNLVAMTAWLATGAYSGANDSNPEGMLYYENRLDSWLALHPDFWLDYKVTPI

SEQ ID NO: 109 - protein, 81 aa, Streptococcus pyogenes
  misc_feature: sda1
NVSKISNNIKASRNGYLFDRSGLIADSLGGRPFRNNLITGTRTQNVGNNDRKGGMQYIENKVLDHIKRNPKVHVYYKATPV

SEQ ID NO: 110 - protein, 44 aa, Artificial Sequence
  Synthetic: Consensus_ss
EEEEEEHHHHHHHHHHHHHHHHHHHHHHHHHHHHHHEEEEEEEE

SEQ ID NO: 111 - protein, 43 aa, Artificial Sequence
  Synthetic: Consensus_aa
PPBSPPSBHRTLLSPHTGSBNLHSTPHHESLSHLHHLYKHPSL